(19) United States
(12) Patent Application Publication
(10) Pub. No.: US 2013/0055471 A1
(43) Pub. Date: Feb. 28, 2013

(54) Transgenic Ozone-Resistant Plants

(71) Applicant: Edwin Henricus Antonius Holman, Ootmarsum (NL)

(72) Inventor: Edwin Henricus Antonius Holman, Ootmarsum (NL)

(21) Appl. No.: 13/515,908

(22) PCT Filed: Dec. 15, 2010
(86) PCT No.: PCT/NL10/50853

§ 371 Date: Sep. 11, 2012

(30) Foreign Application Priority Data
Dec. 15, 2009 (EP) 09075552.1

(51) Int. Cl. A01H 5/00; A01H 5/10; C12N 15/82

(52) U.S. Cl. 800/290; 800/298

(57) ABSTRACT

The invention relates to a transgenic plant comprising a gene that confers resistance to oxidative stress in said plant, wherein said gene encodes an enzyme that has anti-oxidant activity or an enzyme that produces an antioxidant compound, and wherein said gene is under the control of a regulated promoter functional during the early ontogeny of said plant.

### FIELD OF THE INVENTION

The present invention is in the field of crop production. More in particular, the present invention relates to transgenic ozone-resistant plants and to transgenic plants transformed with a gene encoding an enzyme that provides protection against ozone-induced damage, such as the enzyme isoprene synthase. The invention further relates to methods for growing plants and methods for increasing the yield of a plant and to plants produced by the method of the invention exhibiting increased yield.

### BACKGROUND OF THE INVENTION

Global demand for wheat, rice, corn, and other essential grains is expected to steadily rise over the next twenty years. Apart from serving as a food source, the demands will rise as plant-based fuel and chemicals production by biological processes is growing. Meeting this demand by increasing production through increased land use is not very likely; and while better crop management can make a marginal difference, most agriculture experts agree that this anticipated deficit must be made up through increased crop yields.

Modern crop production management systems are tailored to optimize each and every parameter that influences crop yield. Management of moisture and nutrient availability and control of pests is standard in all agricultural production schemes for field crops. In greenhouse crops, availability of CO₂ and light, vapour pressure deficit and temperature can in addition be optimized. Hence, most crops are currently produced under apparently optimal conditions. Although some variation is perceived as “natural”, realistic production efficiencies are still a fraction of theoretical maximum levels. In fact, year-average efficiency levels attained in the field for most crops are often less than half of the maximum levels observed in particular years. The reason for this difference is not properly understood. Hence, there is a need for further understanding and optimizing biological production efficiencies.

Plant biomass production is determined by the plants net carbon fixation efficiency. This efficiency is governed by the rate of carbon gain in terms of CO₂ fixation by photosynthesis and the rate of carbon loss in terms of CO₂ emission by respiration. While gross photosynthesis rises with temperature, so does respiration and whereas the photosynthesis rate tends to flatten at the optimum of the photosynthetic enzyme rubisco of about 25° C., respiration continues to rise rapidly above this temperature and roughly doubles every 10° C. (Q₁₀ is usually 2). At plant-temperatures above about 35° C. (at least for C-3 species) all the sugar produced is used to support respiration. At plant-temperatures higher than 35° C., plants respire more sugar than they can produce which leads to deterioration and ultimate death of the plant. Consequently the net photosynthesis (the production of energy compounds minus their use by respiration including photorespiration) must be considered when attempting to optimize production. As noted above, traditional methods for improving carbon balance are aimed at improving the gross photosynthesis rate. Although a relationship between respiration rate and temperature is well known, and respiration is known to vary depending on protein turnover and maintenance requirements, baseline respiration rates are generally believed to be relatively fixed.

During their life cycle, plants undergo a large number of physiological, biochemical and morphological changes that are controlled by alterations in gene expression. Yet, the morphogenetic capacity of plants is a function of both genetic and environmental parameters, of which thermal and light conditions appear to have the greatest influence. Plant ontogeny, the sum of morphogenic processes that describe the development of a plant from seed germination through to maturity, is known to influence plant production parameters, such as the degree to which plants compensate after defoliation or herbivore damage. Also, it is known that factors that affect crop growth early in ontogeny often produce modifications that extend through the season and can be manifest in altered economic yield. It is believed that this is the result of epigenetic factors, among which DNA-methylation is one of the best known. Plant ontogeny includes developmental changes in plant architecture, storage capacity, and resource allocation to different functions (e.g., storage, defence, reproduction). In the case of woody species, an increase in the plant age is associated with changes in resource allocation patterns, as the carbon/nutrient balance, storage capacity, and access to water and nutrients usually increase, while root to shoot ratio, growth rate, photosynthesis, stomatal conductance, hormone production, and metabolic activity typically decrease. In addition, morphological differences between juvenile and adult trees include variation in leaf morphology, phyllotaxy, shoot orientation, seasonal leaf retention, presence of adventitious roots, and leaf-specific mass.

It is however not known whether crop production in general is influenced by ontogenic processes, or whether factors that affect crops early in ontogeny can produce modifications that extend throughout the life of the plant.

Isoprene (2-methyl-1,3-butadiene) is emitted by a large number of plant species. Isoprene emission generally consumes a significant percentage of the carbon fixed during photosynthesis. The role of isoprene emission in plants is not fully understood. It was suggested to provide protection against heat stress. Indeed, isoprene emissions in some plants exhibit temperature response patterns that are dependent on the plant's growth temperature. Other alkenes are also found to increase thermotolerance in leaves. Isoprene is also suggested to provide a more general protection against stress conditions and in particular against (photo)-oxidative stress and it was recently shown to protect leaves against ozone. The ability of alkenes, such as isoprene, to react with singlet oxygen, ozone and OH radicals is well known, and isoprene emission has been suggested as a quencher of ozone. Thus, although the correlation between leaf temperature and isoprene emission in plants is well known, the physiological role of isoprene emission, quite costly to the plant, is still under debate.

Also vitamin C, ascorbate, is recognized as a factor that protects plants against oxidative stress, such as ozone stress. Further, tartaric acid and lycopene (a tetraterpene consisting of 8 isoprene units) have been proposed as a plant-derived anti-oxidants.

Non-isoprene emitting plants can be rendered isoprene-emitting via transgenesis—the process of introducing an exogenous gene (the transgene) into a living organism so that the organism will exhibit a new property and transmit that property to its offspring. The transgene in this case is isoprene synthase (EC 4.2.3.27), the enzyme of the family of carbon-oxygen lyases acting on phosphates that catalyzes the chemical reaction from dimethylallyl diphosphate (DMAPP) to isoprene and diphosphate.

Such transgenic plants, notably Arabidopsis thaliana plants, have been produced for the purpose of studying the function of isoprene emission and thermal tolerance in plants. However, these transgenic plants have hitherto not been produced for the purpose of improving the plant's characteristics, such as its biomass production capabilities.

Also, plants that overexpress ascorbic acid, tartaric acid or lycopene are known.

It is however not understood how processes for the protection against stress conditions can be made beneficiary so as to increase the yield of said plant. Hence there exists a need for processes and plants exhibiting increased yield despite the presence of yield-lowering processes that are essential to plant survival.

### SUMMARY OF THE INVENTION

The present inventor has now discovered that baseline respiration rate, in particular the AOX component of the total dark respiration, is fixed early in the ontogeny of plants. In fact, the inventor has discovered that oxidative stress early in the ontogeny of plants results in plants having a high baseline respiration rate for the rest of their productive life span. Although in mature plants, the respiration rate can temporarily increase in response to altered environmental conditions, it will return to a rate the level of which is ultimately fixed at an early stage of development.

This discovery was done in Brazil, where annual sugar cane burnings result in high temporary atmospheric concentrations of ozone. It was found that plants that were planted in a period wherein ozone concentrations were lowest consistently exhibited high production, whereas plants that were planted in a period such that the early ontogenic phase coincided with these adverse conditions consistently exhibited low production rates.

Hence, based on these findings the inventor found that plants that are capable of counteracting the adverse effects of atmospheric ozone or that are prevented in any way from encountering these adverse effects during early ontogeny exhibit high production, in terms of dry matter content.

Early in ontogeny plants determine a default respiration rate based on the conditions that prevail at that time in their life. An increase in protein turnover, for instance due to increased tissue damage, will result in a higher respiration rate. It was discovered that in early ontogeny this is essentially a one-way street: the baseline respiration rate can go up, but it cannot come down. As a result, a plant that is exposed to unfavourable conditions such as high ozone concentrations that will potentially result in leaf damage and which plant as a consequence increases its respiration rates early in ontogeny in order to avoid such damage, will keep high baseline respiration rates essentially throughout its life, or at least for prolonged periods of time. Hence, such a plant will exhibit a low net production, as much of the assimilated carbon is respired.

The present inventor has now discovered that plants that are grown in the presence of ozone-protecting compounds, and hence that are protected from the effects of ozone (i.e oxidative stress), in particular at a certain, early, stage in their life, show an enormous increase in biomass production rates and on top of that a higher level of disease-resistance. The presence of ozone-protecting compounds can be achieved by providing specific climatological conditions in a greenhouse, or by producing these compounds in the vicinity of the plant e.g. by micro-organisms or by the plants themselves. The present inventors have discovered that levels or emission rates of ozone-protecting compounds in the vicinity of plants or in transgenic plants must be such that baseline respiration rates, in particular the AOX component thereof, are not elevated by ozone exposure. For isoprene, this is achieved when at least during early ontogeny of the plants, the terpene emission by the plant at 30° C. is at least 0.1-20 nmol·m⁻²·s−1 corresponding to a similar quantity of ozone influx in leaves based on a 1.6-320 ppbv levels of ozone in air. Other suitable minimal emission rates to avoid increase in the baseline respiration rates, in particular the AOX component thereof, are for instance 3-600 μg/g dry weight/h, and 0.0025-0.5 μg/cm2/h of isoprene nmol·m⁻²·s−1 is considered suitable for use in aspects of the invention in order to neutralize the influx of ozone, corresponding to about 1-50% of the carbon assimilated in said plant.

The mitochondrial electron transport chain involves two pathways: the cytochrome pathway and the alternative oxidase (AOX) pathway. In plants, heat stress, and as contemplated herein also drought stress, temperature stress, radiation stress, and salt stress induces the release of cytochrome c (cyt-c) from the respiratory chain, which in turn leads to elevated levels of Reactive Oxygen Species (ROS) and induction of AOX. AOX is a cyanide-resistant, hydroxamic-acid-sensitive terminal oxidase found in the inner mitochondrial membranes of plants, some fungi, and trypanosomes. The activity mediates the non-proton-translocating transfer of electrons from the UQ pool to molecular O₂ to form water. Electrons flowing through AOX bypass proton-translocating complexes III and IV of the Cyt-mediated electron transport chain, causing the oxidative potential energy to be lost as heat. The function of AOX in plants during normal vegetative growth and development remains unclear. The present inventor has now found that the minimum level of the AOX pathway as component of the total dark respiration is determined early in the life of the plant, that is: in the ontogenic phase, and that the respiration efficiency in terms of ATP generated, and therefore the yield of the plants, is less when the plants are exposed early in there life to conditions that elevate the AOX pathway. It has now also been found that this rise in the AOX pathway can be prevented intentionally, by manipulating the plant or its environment during early ontogeny. Such a manipulation is only possible at an effective level when appreciating the present invention.

In a first aspect, the present invention provides a transgenic plant comprising a gene that confers resistance to oxidative stress in said plant, wherein said gene encodes an enzyme that has anti-oxidant activity or an enzyme that produces an antioxidant compound, and wherein (the expression of) said gene is under the control of a regulated promoter functional during the early ontogeny of said plant. Preferably, the promoter is not functional after the early ontogeny of said plant, for instance when the plant is mature.

When said gene is expressed in said plant under conditions that cause periodic oxidative stress, an increase in the rate of respiration and/or protein turnover in said plant due to said periodic oxidative stress can be prevented during the early ontogeny of said plant. Hence, the plant of the present invention is resistant to oxidative stress, preferably periodic oxidative stress, preferably brought about by ozone. In preferred embodiments, the level of expression of the gene that confers resistance to oxidative stress in said plant is such that the oxidative stress is prevented, preferably by neutralization of reactive oxygen species (ROS), in particular ozone.

Due to the presence of this gene, which can suitably be a heterologous gene, the plant can be grown during its early ontogeny in the presence of oxidative stress inducing factors, such as ozone, because an anti-oxidant compound (eg. an ozone-protective compound) is produced in the vicinity of the plant by the plant itself.

Preferably, in this latter option, the plant is transgenic for one or more enzymes that produce said anti-oxidant compound from a substrate that is normally present in said plant. Preferably, the invention provides a transgenic plant comprising a vector for the expression of a (heterologous) isoprene synthase enzyme, said expression vector comprising a nucleotide sequence for the gene encoding the enzyme synthase operably linked to a regulated, preferably inducible, promoter functional in said plant.

Preferably, said plant exhibits an isoprene emission rate of at least 20 nmol·m⁻²·s⁻¹ upon the expression of said vector.

In a preferred embodiment of a transgenic plant of the present invention, the expression of said vector in said plant results in a terpene emission rate between 0.1-500 nmol·m⁻²·s⁻¹, suitably 10-200 nmol·m⁻²·s⁻¹.

A transgenic plant of the present invention is characterized by the fact that it is tolerant to exposure to oxidative stress-inducing conditions during early ontogeny. A transgenic plant of the present invention exhibits an AOX as component of total dark respiration that is essentially equal to a plant of the same variety that has never been exposed to oxidative stress, such as ozone induced oxidative stress. Moreover, a transgenic plant of the present invention exhibits an AOX as component of total dark respiration that is significantly lower (e.g. at least 5-25% lower) than that of a plant of the same variety that was exposed to said same oxidative stress-inducing conditions during its early ontogeny but which is not transgenic as described herein in order to prevent an increase in the AOX. Moreover, the plants have increased dry matter content relative to an exposed plant which, or the environment of which, was not manipulated as indicated herein.

It is a feature of the transgenic plants of the invention that the expression of the transgene that confers tolerance to oxidative stress-inducing conditions by virtue of the production of anti-oxidant compounds is regulated. Although a certain minimum level of AOX respiration can be required for a proper initiation of growth of the seedling and a start of their development, it is undesirable if plants upregulate the AOX component of total dark respiration excessively due to excessive oxidative stress in the early development stages.

It is conceived herein that a certain level of oxidative stress can be needed to prime the development of a germinating and/or germinated seed, and that the selective induction of transgene expression can shortly be postponed until after the germinating and/or germinated seed has been temporarily exposed to oxidative stress-inducing conditions such as ozone. Subsequent induction of transgene expression can then protect the developing seedling from excessively upregulating its AOX which high AOX will compromise its yield for the remainder of its life. Then, following the ontogenic phase, which can suitably have a length of 35-70 days or less for certain crop plants as indicated in the experimental part below, the expression of the transgene can be blocked or induction can be terminated, which blockage or termination has as an advantage that the production of anti-oxidant compounds (such as isoprene) can be stopped once their role has ended in order not to loose fixed carbon and favor further yield increase.

In another preferred embodiment of a transgenic plant of the present invention, said plant is from a species that does not naturally emit isoprene. Such a plant is herein referred to as a plant from a non-terpene emitting plant species, which term includes reference to plants of which the terpene emission rates are to be increased if an increase in the AOX during early ontogeny as a result of periodic oxidative stress is to be prevented by conversion of ozone. A plant from a species that does not naturally emit terpene thus also includes reference to a low-terpene emitter which can benefit from the claimed invention.

In an alternative preferred embodiment, the present invention provides a transgenic plant comprising a vector for the expression of one or more enzymes that produce an anti-oxidant compound in a plant, said vector preferably comprising a, preferably heterologous, nucleotide sequence for a gene encoding the enzyme isoprene synthase operably linked to an inducible promoter functional in said plant, and wherein said expression of said gene results in the emission of an anti-oxidant compound, preferably isoprene, lycopene, tartaric acid or ascorbic acid, at a rate sufficient to protect said plant from damage due to oxidative stress brought about by reactive oxygen species, including H₂O₂ or O₃, preferably O₃.

In a preferred embodiment of the plant of the invention, said inducible promoter ensures that said gene is expressed in said plant at least during early ontogeny of said plant. More preferably the inducible promoter ensures that said gene is not expressed after the early ontogeny of said plant or plant tissue. This helps to reduce unnecessary carbon losses in the form of isoprene emissions at a stage wherein the protective effect, although still helpful with respect to counteracting direct damage due to oxidative stress, is not essential to the gross productivity of the plant in the long term, because this oxidative stress does no longer affect the basic respiration rate of said plant under normal conditions or because the tissues have already fixed their maximum amount of carbon.

Alternatively, instead of an inducible promoter, a promoter that is predominantly active during the early ontogeny of the plant can be used.

In an alternatively preferred embodiment, said plant is not a transgenic Ginseng (Panax ginseng), Kudzu (Pueraria montana), Tobacco (Nicotiana tabacum), Grey poplar (Populus×canescens), Populus alba, or Arabidopsis thaliana plant.

In another preferred embodiment of the plant of the invention, said early ontogeny is the period between germination and the generative phase, thus preferably the vegetative phase or stage, that is before the plants induce and develop flowers. This stage can be anywhere between a few days to 0.5-6 months post germination, more preferably about 70 days or less, and is generally longer for trees than for crop plants.

In an alternatively preferred embodiment of a plant of the invention said inducible promoter or said ontogenesis-specific promoter ensures that said gene is expressed in said plant at least during early ontogeny of (the leaves of) said plant. In addition, in a preferred embodiment said inducible promoter or said ontogenesis-specific promoter ensure that said gene is not expressed in said plant after full development of green vegetative tissues or after said early ontogenic phase, that is for instance during the generative phase wherein said plant develops flowers, seeds and fruits or after leaves have reached their maximum dry weight.

Preferably the transgenic plant exhibits a baseline respiration rate (including and preferably with reference to the alternative oxidase pathway) as the rate that is at most 80%, preferably at most 70, 60, 50, 40, 30, or even 20% of that of a plant of the same species that is not transgenic and that does not contain the vector as described herein and which has consequently not been subjected to ozone-protective effect of the vector as described herein, at least during early ontogeny of (the leaves of) said plant. In a highly preferred embodiment this early ontogeny is the period 15-60 days after emergence (DAE) of the first leaf of said plant. Alternatively, in another highly preferred embodiment this early ontogeny is the period coinciding with the pre-floral stage of said plant.

In another preferred embodiment of the plant of the invention, the expression of said vector in said plant results in an isoprene emission rate of at least 0.1 nmol isoprene·m⁻²·s⁻¹, preferably between 10-40 nmol·m⁻²·s⁻¹.

In yet another preferred embodiment of the plant of the invention, said inducible promoter is part of a so-called switch system, preferably selected from the group consisting of the alcA/alcR gene switch promoter; the GST promoter, and the ecdysone switch system.

In yet another preferred embodiment of the plant of the invention, said gene is expressed in the leaves of said plant.

In still another preferred embodiment of the plant of the invention, said plant is from a species that does not naturally produce (in any significant or effective quantity) one of the anti-oxidant compounds mentioned above.

In still another preferred embodiment of the plant of the invention, said plant is an adult plant, and said adult plant exhibits a first rate of respiration under ambient ozone that is essentially equal to the rate of maintenance respiration exhibited by said plant during early ontogeny, and wherein said plant upon temporary exposure to ambient plus 50 or 100 ppbv of ozone exhibits a second rate of respiration, which second rate is a significant increase relative to said first rate, and wherein following said temporary exposure said second rate of respiration returns to pre-exposure levels.

In an even more preferred embodiment of this plant said rate of maintenance respiration is the rate of respiration of a young full grown leaf of a plant measured when said plant has not been exposed to ozone, preferably said rate of respiration refers to the AOX component in de total (dark) respiration due to oxidative stress as a result of which the respiration loses efficiency in terms of generating ATP. Thus, preferably the AOX in a plant of the invention that has passed the ontogenic phase and that has been exposed to AOX-enhancing levels of ozone, is close to zero, or at least close to that of a plant never exposed to ozone or other oxidative stress. Thus, plants of the invention when placed in an oxidative-stress-free environment have the ability of having their AOX essentially return to 0% (or pre-exposure levels). Whereas plants that have not been grown by a method of the invention and that have been exposed to oxidative stress during the ontogenic phase have relatively high AOX components even when thereafter placed in an oxidative-stress-free environment.

In another aspect, the present invention provides a progeny plant or seed from the transgenic plant of any one of the preceding claims, wherein said progeny plant or seed comprises said (heterologous) gene under the control of said regulated promoter.

In another aspect, the present invention provides a seed from the progeny plant of the invention as described above, wherein said seed comprises said (heterologous) gene under the control of said regulated promoter.

In another aspect, the present invention provides a plant from the seed of the invention as described above, wherein said plant comprises said (heterologous) gene under the control of said regulated promoter.

In another aspect, the present invention provides a method of preparing a transgenic plant having improved yield under conditions of periodic oxidative stress, said method comprising the steps of:

(a) obtaining a nucleic acid segment comprising a gene, preferably a gene that is heterologous to said plant, wherein said gene encodes an enzyme that has anti-oxidant activity or an enzyme that produces an antioxidant compound, and wherein said gene is operably linked to a regulated promoter functional during the early ontogeny of said plant;
(b) transforming a plant cell with said nucleic acid segment; and
(c) regenerating from said plant cell a transgenic plant which expresses said (heterologous) gene and wherein said transgenic plant exhibits improved yield under conditions of periodic oxidative stress as compared to a non-transformed plant.

In a preferred embodiment of this method of preparing a transgenic plant of the invention said step a) further comprises introducing said nucleic acid segment into a vector, and wherein step b) comprises transforming said plant cell with said vector. In preferred embodiments said vector is a phage vector, bacterial vector, a plasmid vector or viral vector.

In another preferred embodiment of this method of preparing a transgenic plant of the invention said oxidative stress is caused by drought, temperature, radiation, salt and/or exposure to reactive oxygen species, preferably ozone.

In yet another preferred embodiment of this method of preparing a transgenic plant of the invention, said regulated promoter is an inducible promoter, a developmentally regulated promoter, and/or a tissue-specific promoter. Preferably said regulated promoter does not express said gene after the early ontogeny of said plant.

In a preferred embodiments said inducible promoter is selected from the alcA/alcR gene switch promoter, the GST promoter, and the ecdysone switch system.

In yet other preferred embodiments of this method of the invention, said developmentally regulated promoter is an ontogenesis-specific promoter selected from the Pyk10 promoter from Arabidopsis thaliana, the malate synthase promoter from Brassica napus, the isocitrate lyase promoter from Brassica napus, the promoter of the GSBF1 gene from Brassica napus, the glycine-rich RNA binding protein gene of Oryza sativa, the cysteine protease gene promoter of Brassica napus, the promoters of lipid transfer protein genes from Hordeum vulgare, and homologues thereof in other plant species. In a preferred embodiments said inducible promoter is a developmentally regulated promoter

In alternatively preferred embodiments, said tissue-specific promoter is selected from the promoter of the isoprene synthase gene from Populus (e.g. the ISPS promoter); the rbcS (Rubisco) promoter from Coffea, Brassica, Chrysanthemum, Phaseolus and Glycine max; the cy-FBPase promoter; the promoter sequence of the light-harvesting chlorophyll a/b binding protein from Elaeis; the STP3 promoter from Arabidopsis thaliana; the promoter of the PAL2 gene from Phaseolus; the enhancer sequences of the ST-LS1 promoter from Solanum tuberosum; the CAB1 promoter from Triticum; the stomata-specific promoter from the ADP-glucose-phosphorylase gene from Solanum tuberosum; the LPSE1 element from the P(D540) gene of Oryza sativa; and the stomata specific promoter pGC/(At1g22690) from Arabidopsis thaliana, and homologues in other plant species.

In addition, combinations of promoters as indicated above can be used. For instance, a tissue-specific promoter, such as the Grey Poplar Isoprene Synthase promoter (PcISPS promoter) in combination with an inducible promoter e.g. the alcA/alcR gene switch promoter or GST promoter, will provide for very advantagoues site-specific and temporally regulated expression of the enzyme. For instance, a developmentally regulated promoter is combined with a ozon regulated promoter to drive expression of a terpene synthase such as isoprene synthase in developing or differentiating tissues during an ozone event, which event coincides with atmospheric ozone levels that activate the ozon regulated promoter.

In further alternatively preferred embodiments, said enzyme that produces an antioxidant compound is selected from the group consisting of isoprene synthase, glutathione reductase, dehydroascorbate reductase, L-galactono-γ-lactone dehydrogenase, phosphomannomutase, GDP-D-mannose pyrophosphorylase (GMP), GDP-mannose-3′,5′-epimerase, L-galactono-1,4-lactone dehydrogenase, Gal-UR, the gene encoding miox4, and L-idonate dehydrogenase.

In yet further alternatively preferred embodiments, said enzyme that has anti-oxidant activity is selected from the group consisting of glutathione peroxidase, glutathione reductase, catalase, thioredoxin reductase, superoxide dismutase, heme oxygenase and biliverdin reductase.

In preferred embodiments of aspects of the invention said early ontogeny corresponds to the prefloral stage or the vegetative stage, preferably the period between germination and 0.5-6 months post germination.

In preferred embodiments of aspects of the invention said (heterologous) gene is expressed in the plastids of said plant.

In highly preferred embodiments of aspects of the invention the gene is the isoprene synthase gene.

In further highly preferred embodiments of aspects of the invention vector in said plant results in a terpene emission rate of at least 0.1 nmol·m⁻²·s⁻¹, preferably between 10-40 nmol·m⁻²·s⁻¹.

The plant in aspects of the present invention is preferably from a species that does not naturally emit isoprene. More preferably it is a row crop plant.

In another aspect, the present invention provides a transgenic plant obtained by the method according to the invention as described above, wherein said plant comprises said (heterologous) gene under the control of said regulated promoter.

In another aspect, the present invention provides a transgenic seed from the plant obtained by a method of the invention, wherein said seed comprises said (heterologous) gene under the control of said regulated promoter.

In another aspect, the present invention provides a transgenic plant from the transgenic seed from the plant obtained by a method of the invention, wherein said plant comprises said (heterologous) gene under the control of said regulated promoter and wherein said plant, when grown from said seed to maturity under periodic conditions that cause oxidative stress, exhibits a total dark respiration and/or respiration via the alternative oxidase (AOX) pathway that is significantly less as compared to a non-transformed plant. Said transgenic plant preferably exhibits a rate of respiration via the alternative oxidase (AOX) pathway that is below 40%, preferably below 30% of the total dark respiration of said plant. Alternatively, it cyan be said that such a plant exhibits a base-line rate of respiration via the alternative oxidase (AOX) pathway that is below 40%, preferably below 30% of the total dark respiration of said plant, since the base-line respiration is respiration in the absence of oxidative stress inducing factors.

In another aspect, the present invention provides a method of growing plants, comprising the step of allowing a seed, a seedling, tissue culture or plantlet of the plant of the invention as described above to develop into a plant, and inducing expression of said gene during early ontogeny of said plant and/or during periodic conditions that cause oxidative stress, to thereby prevent an increase in the rate of respiration via the alternative oxidase (AOX) pathway during said early ontogeny and/or due to said oxidative stress.

In preferred embodiments of said method of growing plants, said induction is brought about by contacting said seed, seedling, tissue culture, plantlet or plant with an effective concentration of a promoter-inducing agent.

In preferred embodiments of aspects of the invention said plant is a perennial plant or a crop plant, most preferably wheat, corn, melon, soy, potato, rice, sugarcane, sugarbeet, evening primrose, meadow foam, hops, jojoba, peanuts, safflower, barley, oats, rye, wheat, sorghum, tobacco, kapok, beans, lentils, peas, soybeans, rape, mustard, poppy, olives, sunflowers, coconut, castor oil plants, cocoa beans, groundnuts, cotton, flax, hemp, jute, cinnamon, tomato, cucumber, pepper, camphor, coffee, sugarcane, tea, rubber plant.

In preferred embodiments of said method of growing plants, said method further comprises the step of discontinuing said induction when said plant reaches the floral stage or the generative stage or when said periodic conditions that cause oxidative stress are absent.

In preferred embodiments of said method of growing plants, said method further comprises determining prior to or simultaneously to growing said seed, seedling, tissue culture, plantlet or plant:

(a) the total dark respiration and/or respiration via the alternative oxidase (AOX) pathway in said plant or a plant of the same variety;
(b) the length of the early ontogenic phase in said plant or a plant of the same variety; and/or
(c) the length and interval of the periodic conditions that cause oxidative stress; and using said information in order to induce expression of said gene during early ontogeny of said plant and/or during said periodic oxidative stress during the early ontogeny of said plant, but not during maturity of said plant or during the generative phase.

In another aspect, the present invention provides a method of growing plants, comprising growing the transgenic plants of the invention.

In another aspect, the present invention provides a method for increasing the yield of a plant, comprising expressing a (heterologous) enzyme in said plant comprising: introducing into a plant cell, a first construct containing a nucleotide sequence encoding the (heterologous) enzyme and operably linked thereto, a regulated promoter active in plants that is activated during the early ontogenic phase of said plant, and regenerating the plant from said cell to provide a transgenic plant. Although this transgenic plant can be used directly in a method of the invention, the plant is preferably grown to seed maturation. The transgenic seed is subsequently used for the production of crops. The transgenic crop plants are then exposed during the early ontogeny of said plant to conditions that induce expression of the transgene in said plant. Prior and/or during the early ontogenic phase, the transgenic plants are then suitably dusted or sprayed with inducing chemicals (also referred to as switch chemicals herein) so as to effect promoter activation in said plants during the early ontogenic phase. The skilled person will understand that for each plant species, and even for different plant varieties, the onset and duration of the early ontogenic phase can differ. As a suitable duration, a spraying or dusting of about 1-10 weeks from germination, preferably shorter, is envisioned.

A method for increasing the yield of a plant includes reference to methods for increasing stress tolerance as indicated herein and to methods for increasing disease resistance in said plant.

In embodiments of this method, the oxidative stress is the stress in a plant that can be brought about by radiation, heat, salt, drought, SO₂, low CO₂, or reactive oxygen species, including H₂O₂, or O₃, preferably H₂O₂ or O₃, most preferably O₃ or precursors of O₃, such as NOx.

In another preferred embodiment of the method of the invention, the oxidative stress is prevented by controlling the content of a reactive oxygen species (ROS) and their precursors (e.g. NOx) in the growth environment. Said reactive oxygen species in the growth environment can be H₂O₂, or O₃, preferably H₂O₂ or O₃, and is most preferably O₃ in the growth atmosphere. The ROS content can be controlled in one of many ways. For instance in a closed system, it can be controlled by using ROS scrubbers. A suitable ROS scrubber is for instance an activated carbon filter. Alternatively, the ROS and particularly the O₃ content in the growth atmosphere can be controlled by exposing the plant or its growth atmosphere to terpenes. Isoprene is known to neutralize ozone. In a further preferred embodiment, the terpenes can be produced in leaves of a transgenic or recombinant plant transformed with a gene encoding an enzyme terpene synthase.

In yet another preferred embodiment of the method of the invention, the plant is a perennial plant and/or a crop plant, more preferably a row crop and/or open field crop plant.

In another aspect, the present invention provides a method for increasing the average yearly crop production of a crop production area, comprising growing a plant according to (a method of) the invention as described above and using said plant as a crop plant in said production area.

In a preferred embodiment of this method, the increase in the average yearly crop production is brought about by an improved net carbon fixation efficiency, biomass production, dry matter content and/or pest resistance of said crop plant.

In yet another aspect, the present invention provides a plant part obtained from a plant of the present invention as described above.

In yet another aspect, the present invention provides a method for optimizing the production of a plant, comprising the steps of:

a) providing a transgenic plant by transforming said plant with a vector comprising a nucleic acid sequence encoding an enzyme capable of converting reactive oxygen species into an non-reactive or harmless (non-damaging) form or with a vector comprising a nucleic acid sequence encoding an enzyme that, when expressed in cells of said transgenic plant, result in the production of an anti-oxidant compound, wherein said nucleic acid sequence is operably linked to a regulated promoter functional during early ontogeny in said specific type of plant; and

b) producing transgenic seed from said transgenic plant and sowing a transgenic seed or planting a transgenic seedling or plantlet obtained therefrom and allowing the seedling or plantlet to develop into a plant, wherein during said early ontogenic phase of said transgenic plant said regulated promoter is activated.

### SHORT DESCRIPTION OF THE DRAWINGS

FIG. 1 shows the measurement results of phase shift fluorescence measurement as described in Example 2 for cotton plants grown (and permanently residing under 0 ppbv ozone in phytotron 1 (grey bar (left): SHAM inhibited sample; black bar (right): uninhibited control).

FIG. 2 shows the graphic presentation of total dark respiration as measured by phase shift fluorescence as a function of ozone in Cotton full leaf samples. A: Relative respiration (TDR) per fresh weight versus Ozone//3 ppbv=100%. (See also Table 2); B: Relative respiration (TDR) per dry weight versus Ozone (See Table 2).

FIG. 3 shows the graphic presentation of total dark respiration as measured by phase shift fluorescence as a function of ozone in Rice full leaf samples. A: Relative respiration (TDR) per fresh weight versus Ozone//3 ppbv=100%. (See also Table 3); B: Relative respiration (TDR) per dry weight versus Ozone (See Table 3); C: Dry matter content versus Ozone.

FIG. 4 shows the graphic presentation of total dark respiration as measured by phase shift fluorescence as a function of ozone in Corn full leaf samples. A: Relative respiration (TDR) per fresh weight versus Ozone//3 ppbv=100%. (See also Table 4); B: Relative respiration (TDR) per dry weight versus Ozone (See Table 4).

FIG. 5 shows the results of AOX measurements in sugarcane as described in Example 3.

### DETAILED DESCRIPTION OF THE INVENTION

### Definitions

The term “gene” is used broadly to refer to any segment of nucleic acid associated with a biological function. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include nonexpressed DNA segments that, for example, form recognition sequences for other proteins. Genes can be obtained from a variety of sources, including cloning from a source of interest or synthesizing from known or predicted sequence information, and can include sequences designed to have desired parameters.

The term “native” or “wild type” gene refers to a gene that is present in the genome of an untransformed cell, i.e., a cell not having a known mutation. The term “native” or “wild type” is intended to encompass allelic variants of the gene.

A “marker gene” encodes a selectable or screenable trait. The term “selectable marker” refers to a polynucleotide sequence encoding a metabolic trait which allows for the separation of transgenic and non-transgenic organisms and mostly refers to the provision of antibiotic resistance. A selectable marker is for example the aphL1 encoded kanamycin resistance marker, the nptII gene, the gene coding for hygromycin resistance. Other selection markers are for instance reporter genes such as chloramphenicol acetyl transferase, β-galactosidase, luciferase and green fluorescence protein. Identification methods for the products of reporter genes include, but are not limited to, enzymatic assays and fluorimetric assays. Reporter genes and assays to detect their products are well known in the art and are described, for example in Current Protocols in Molecular Biology, eds. Ausubel et al., Greene Publishing and Wiley-Interscience: New York (1987) and periodic updates.

The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene can comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and preferably is stably maintained. Transgenes can include, for example, genes that are either heterologous or homologous to the genes of a particular plant to be transformed. Additionally, transgenes can comprise native genes inserted into a non-native organism, or chimeric genes. Additionally, the term transgene may include reference to autologous genes placed under the control of a heterologous or homologous promoter. The term “endogenous gene” refers to a native gene in its natural location in the genome of an organism. A “foreign” gene refers to a gene not normally found in the host organism but that is introduced by gene transfer.

An “oligonucleotide”, e.g., for use in probing or amplification reactions, can be about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16 to 24 nucleotides in length can be preferred. Those skilled in the art are well versed in the design of primers for use in processes such as PCR. If required, probing can be done with entire restriction fragments of the gene disclosed herein which can be 100's or even 1000's of nucleotides in length.

The terms “protein”, “peptide” and “polypeptide” are used interchangeably herein.

“Coding sequence” refers to a DNA or RNA sequence that codes for a specific amino acid sequence and excludes the non-coding sequences. It can constitute an “uninterrupted coding sequence”, i.e., lacking an intron, such as in a cDNA or it can include one or more introns bound by appropriate splice junctions. An “intron” is a sequence of RNA which is contained in the primary transcript but which is removed through cleavage and re-ligation of the RNA within the cell to create the mature mRNA that can be translated into a protein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which can be a combination of synthetic and natural sequences. As is noted above, the term “suitable regulatory sequences” is not limited to promoters.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and can be an innate element of the promoter or a (heterologous) element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters can be derived in their entirety from a native gene, or be composed of different elements derived from different promoters found in nature, or even be comprised of synthetic DNA segments. A promoter can also contain DNA sequences that are involved in the binding of protein factors which control the effectiveness of transcription initiation in response to physiological or developmental conditions.

The term “functional”, with reference to a promoter, as used herein, refers to a promoter having a functional promoter sequence such that it can drive the expression of the gene to which it is operatively linked. The term functional promoter sequence refers to a sequence of nucleotides that can be recognized by an RNA polymerase and from which gene transcription can be initiated.

The “initiation site” is the position surrounding the first nucleotide that is part of the transcribed sequence, which is also defined as position+1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

“Constitutive expression” refers to expression using a constitutive promoter.

“Conditional” and “regulated expression” refer to expression controlled by a regulated promoter.

Developmentally regulated refers to a type of gene expression which is controlled by the age of a specific tissue or by the formation of a specific tissue. A gene that is expressed only in the seedling, young plant, medium aged plant, or early bolting plant, and optionally even in young flowers, and not in a plant at late flowering stage such as in mature flowers or for instance not in mature siliques, is said to be developmentally regulated herein.

“Constitutive promoter” refers to a promoter that is able to express the open reading frame (ORF) that it controls in all or nearly all of the plant tissues during all or nearly all developmental stages of the plant.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Regulated promoter” refers to promoters that direct gene expression not constitutively, but in a temporally- and/or spatially-regulated manner, and includes both tissue-specific, growth-phase-specific and inducible promoters. It includes natural and synthetic sequences as well as sequences which can be a combination of synthetic and natural sequences. Different promoters can direct the expression of a gene in different tissues or cell types, or at different stages of development, or in response to different environmental conditions. New promoters of various types useful in plant cells are constantly being discovered, numerous examples can be found in the compilation by Okamuro et al. (1989). Typical regulated promoters useful in plants include but are not limited to developmentally regulated promoters, safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysone-inducible systems.

“Inducible promoter” refers to those regulated promoters that can be turned on in one or more cell types by an external stimulus, such as a chemical, light, hormone, stress, or a pathogen.

Several inducible promoters (also called “gene switches”) have been reported. Many are described in the review by Gatz (Current Opinion in Biotechnology, 1996, vol. 7, 168-172; Gatz, C. Chemical control of gene expression, Annu. Rev. Plant Physiol. Plant Mol. Biol. (1997), 48, 89-108). These include tetracycline repressor system, Lac repressor system, copper-inducible systems, salicylate-inducible systems, glucocorticoid-(Aoyama T. et al. 1997 N—H Plant Journal 11:605-612) and ecdysome-inducible systems. Also, included are the benzene sulphonamide-(U.S. Pat. No. 5,364,780) and alcohol-(WO 97/06269 and WO 97/06268)-inducible systems and glutathione S-transferase promoters. U.S. Pat. No. 6,063,985 describes glucocorticoid-inducible promoters which can also suitably be used herein. Other suitable examples include genes inducibly regulated in response to environmental stress or stimuli such as increased salinity, drought, pathogen, and wounding (Graham et al. 1985 J. Biol. Chem. 260:6555-6560 and 6561-6554; Smith et al. 1986 Planta 168:94-100). Other plant genes have been reported to be induced methyl jasmonate, elicitors, heat-shock, anaerobic stress, or herbicide safeners. (See also list of Table 16.1 in A. E. Müller and M. Wassenegger, in: Handbook of Plant Biotechnology, Eds: P. Christou and H. Klee, John Wiley & Sons, Chichester, 2004, pag. 294-295).

One preferred example of gene switch promoters include the alcA/alcR gene switch promoter as described in WO 93/21334; the GST promoter, as described in WO 90/08826 and WO 93/031294; and the ecdysone switch system as described in WO 96/37609. In such switch systems, the timing of gene expression is controlled by application of an external chemical. The switch chemical can be applied as a spray or vapour to all or part of the transgenic plant or as a root drench or submersion fluid. Examples of suitable switch chemicals are provided in the above references describing switch promoter systems. The external chemical stimulus is preferably an agriculturally acceptable chemical, the use of which is compatible with agricultural practice and is not detrimental to plants or mammals. Inducible switch promoter systems preferably include one or two component systems; nevertheless, systems comprising more than two components are encompassed by the present invention. The switch system can be driven by a constitutive promoter or by a tissue or organ-specific promoter, whereby the target gene is only switched on in a target tissue or organ. The alcA/alcR switch promoter system is particularly preferred. In the alcA/alcR promoter switch system, the preferred chemical inducer is ethanol, in either liquid or vapour form. One of the main advantages of the use of ethanol is that small quantities of ethanol generate high levels of expression. The alcA/alcR inducible promoter system is a two-component system involving DNA sequences coding for the alcA promoter and the alcR protein, the expression of which is placed under the control of desired promoters. The alcR protein activates the alcA promoter in the presence of an inducer and any gene under the control of the alcA promoter will therefore be expressed only in the presence of that inducer.

Another very suitable promoter includes an ozone-inducible promoter as this will result in an expression of the vector of the present invention when the plant (as seed, seedling, plantlet or full grown (mature) plant) is exposed to ozone (i.e. particularly those conditions for which the vector aims to provide protection. Ozone-inducible promoters are for instance described in WO2001/059139 (in particular the promoter indicated therein under SEQ ID NO:1). Other suitable examples include the −1314 SWPA2 promoter (Kim et al. 2003 Plant Molecular Biology 51(6): 831-838), the promoter region of the Vst1 gene (U.S. Pat. No. 6,740,749, and in particularly the DNA sequence described therein as SEQ ID NO:1, the ozone-inducible promoter of Vitis vinifera VST-1 gene), and the ozone-inducible promoters of the isoprene synthase genes of.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells).

Another type of regulated promoter is a promoter that is active during a limited developmental phase, so-called developmentally regulated promoters. These also include promoters that are temporally regulated, such as in early or late embryogenesis, during fruit ripening in developing seeds or fruit, in fully differentiated leaf, or at the onset of senescence. Especially preferred in the present invention are promoters that are mainly active during the early ontogenetic phase of the plant. One of such promoters is the Pyk10 promoter from Arabidopsis thaliana (Nitz, I. et al., 2001, Plant Science 161: 337-346).

The specificity of a promoter is often caused by a subarea of the promoter. These subareas are interchangeable between various promoters, thereby conferring a specificity from one promoter to another promoter. It is also possible to couple these subareas, such that a coupling of a (part of a) tissue-specific promoter with a (part of a) development specific promoter will yield a promoter that is specific for both a tissue and a developmental stage. In this way, a promoter fitted for a specialized function can be assembled.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, ORF or portion thereof, or a transgene in plants. Expression refers to the transcription and stable accumulation of sense (mRNA) or functional RNA. Expression can also refer to the production of protein.

“Specific expression” is the expression of gene products which is limited to one or a few plant tissues (spatial limitation) and/or to one or a few plant developmental stages (temporal limitation). It is acknowledged that hardly a true specificity exists: promoters seem to preferably switch on in some tissues, while in other tissues there can be no or only little activity. This phenomenon is known as leaky expression. However, with tissue-specific expression in this invention is meant preferable expression in one or a few plant tissues.

The terms “heterologous DNA sequence”, “exogenous DNA segment” or “heterologous nucleic acid”, as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Homologous to” in the context of nucleotide or amino acid sequence identity refers to the similarity between the nucleotide sequence of two nucleic acid molecules or between the amino acid sequences of two protein molecules. Estimates of such homology are provided by either DNA-DNA or DNA-RNA hybridization under conditions of stringency as is well understood by those skilled in the art (as described in Haines and Higgins (eds.), Nucleic Acid Hybridization, IRL Press, Oxford, U. K.), or by the comparison of sequence similarity between two nucleic acids or proteins. Two nucleotide or amino acid sequences are homologous when their sequences have a sequence similarity of more than 60%, preferably more than 70%, 80%, 85%, 90%, 95%, or even 98%.

The term “autologous” refers to the fact that the gene or gene product is derived or transferred from the same plant species, i.e. relating to the naturally occurring gene or gene product in the same or a different type of tissue or in a specific structure of the plant.

The term “substantially similar” refers to nucleotide and amino acid sequences that represent functional and/or structural equivalents of sequences disclosed herein. For example, altered nucleotide sequences which simply reflect the degeneracy of the genetic code but nonetheless encode amino acid sequences that are identical to a particular amino acid sequence are substantially similar to the particular sequences. In addition, amino acid sequences that are substantially similar to a particular sequence are those wherein overall amino acid identity is at least 65% or greater to the instant sequences. Modifications that result in equivalent nucleotide or amino acid sequences are well within the routine skill in the art. Moreover, the skilled artisan recognizes that equivalent nucleotide sequences encompassed by this invention can also be defined by their ability to hybridize, under low, moderate and/or stringent conditions (e.g., 0.1×SSC, 0.1% SDS, 65° C.), with the nucleotide sequences that are within the literal scope of the instant claims.

The term “transformation” refers to the transfer of a nucleic acid fragment into the genome of a host cell (i.e. into the chromosome or extrachromosomally), generally resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., 1987 Methods Enzymol. 153, 277) particle bombardment technology (Klein et al. 1987 Nature 327, 70; U.S. Pat. No. 4,945,050), microinjection, calcium phosphate precipitation, lipofection (liposome fusion), use of a gene gun and DNA vector transporter (Wu et al., 1992 J. Biol. Chem., 267: 963-967). Whole plants can be regenerated from transgenic cells by methods well known to the skilled artisan (see, for example, Fromm et al., 1990 Bio/Technology 8:833-839).

“Transformed”, “transgenic” and “recombinant” refer to a host organism such as a plant into which a (heterologous) nucleic acid molecule has been introduced. The (heterologous) nucleic acid molecule can be stably integrated into the genome by methods generally known in the art. “Transformed”, “transformant”, and “transgenic” plants or calli have been obtained through the transformation process and contain a foreign gene integrated into their chromosome, or residing on an extrachromosomal vector. The term “untransformed” refers to normal plants that have not been through the transformation process.

“Transiently transformed” refers to cells in which transgenes and foreign DNA have been introduced (for example, by such methods as Agrobacterium-mediated transformation or biolistic bombardment), but not selected for stable maintenance.

“Stably transformed” refers to cells that have been selected and regenerated on a selection media following transformation.

“Genetically stable” and “heritable” refer to chromosomally-integrated genetic elements that are stably maintained in the plant and stably inherited by progeny through successive generations.

“Chromosomally-integrated” refers to the integration of a foreign gene or DNA construct into the host DNA by covalent bonds. Where genes are not “chromosomally integrated” they can be “transiently expressed”. Transient expression of a gene refers to the expression of a gene that is not integrated into the host chromosome but functions independently, either as part of an autonomously replicating plasmid or expression cassette, for example, or as part of another biological system such as a virus.

“Primary transformant” refers to transgenic plants that are of the same genetic generation as the tissue which was initially transformed (i.e., not having gone through meiosis and fertilization since transformation).

“Secondary transformants” and the “T1, T2, T3, etc. generations” refer to transgenic plants derived from primary transformants through one or more meiotic and fertilization cycles. They can be derived by self-fertilization of primary or secondary transformants or crosses of primary or secondary transformants with other transformed or untransformed plants. Secondary transformants are an aspect of the present invention.

“Genome” refers to the complete genetic material of an organism.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al., 1991; Ohtsuka et al., 1985; Rossolini et al. 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid sequence” can also be used interchangeably with gene, cDNA, DNA and RNA encoded by a gene.

The nucleotide sequences used in aspects of the invention include both the naturally occurring sequences as well as mutant (variant) forms and homologues. Such variants will continue to possess the desired activity, i.e., either promoter activity or the activity of the product encoded by the open reading frame of the non-variant nucleotide sequence.

The term “homologue” is intended to indicate a natural variant of the promoter or gene sequences indicated herein or a variant or fragment produced by modification of the DNA sequence. Examples of suitable modifications of the DNA sequence are nucleotide substitutions which do not give rise to a change in promoter functionality or which do not give rise to another amino acid sequence or nucleotide substitutions which do give rise to a different amino acid sequence and therefore, possibly, a different protein structure. Other examples of possible modifications are insertions of one or several nucleotides into the sequence, addition of one or several nucleotides at either end of the sequence, or deletion of one or several nucleotides at either end or within the sequence. Any homologous DNA sequence which exhibits the intended activity (e.g., with respect to promoter function or ability to confer resistance to reactive oxygen species) similar to that of the naive sequence is contemplated for use in the claimed invention. Homologues preferably have a nucleotide sequence identity of more than 70%, preferably more than 80%.

Thus, by “variant” is intended a substantially similar sequence. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence.

The term “nucleotide sequence identity” or “nucleotide sequence homology” as used herein denotes the level of similarity, respectively the level of homology, between two polynucleotides. Polynucleotides have “identical” sequences if the sequence of nucleotides in the two sequences is the same. Polynucleotides have “homologous” sequences if the sequence of nucleotides in the two sequences is the same when aligned for maximum correspondence. Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” or “percentage of sequence homology” for polynucleotides, such as 50, 60, 70, 80, 90, 95, 98, 99 or 100 percent sequence identity or homology can be determined by comparing two optimally aligned sequences over a comparison window, wherein the portion of the polynucleotide sequence in the comparison window can include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology. Optimal alignment of sequences for comparison can be conducted by computerized implementations of known algorithms, or by visual inspection. Readily available sequence comparison and multiple sequence alignment algorithms are, respectively, the Basic Local Alignment Search Tool (BLAST) (Altschul et al., 1990 J. Mol. Biol. 215:403-410) and ClustalW programs, both available on the internet. Other suitable programs include, but are not limited to, GAP, BestFit, PlotSimilarity, and FASTA in the Wisconsin Genetics Software Package (Genetics Computer Group (GCG), Madison, Wis., USA).

The nucleic acid sequences of the invention can be “optimized” for enhanced expression in plants of interest. See, for example, EP O₃₅₉₄₇₂ or WO 91/16432. In this manner, the open reading frames in genes or gene fragments can be synthesized utilizing plant-preferred codons. Thus, the nucleotide sequences can be optimized for expression in any plant.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants can result from, for example, genetic polymorphism or from human manipulation. Methods for such manipulations are generally known in the art.

Thus, the polypeptides can be altered in various ways including amino acid substitutions, deletions, truncations, and insertions. Methods for such manipulations are generally known in the art. Conservative substitutions, such as exchanging one amino acid with another having similar properties, are preferred. Individual substitutions deletions or additions that alter, add or delete a single amino acid or a small percentage of amino acids (typically less than 5%, more typically less than 1%) in an encoded sequence are “conservatively modified variations”, where the alterations result in the substitution of an amino acid with a chemically similar amino acid.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but can also code for a functional RNA of interest, for example antisense RNA or a nontranslated RNA, in the sense or antisense direction. The expression cassette comprising the nucleotide sequence of interest can be chimeric, meaning that at least one of its components is heterologous with respect to at least one of its other components. The expression cassette can also be one which is naturally occurring but has been obtained in a recombinant form useful for heterologous expression. The expression of the nucleotide sequence in the expression cassette can be under the control of a constitutive promoter or of an inducible promoter which initiates transcription only when the host cell is exposed to some particular external stimulus. In the case of a multicellular organism, the promoter can also be specific to a particular tissue or organ or stage of development.

The term “vector” as used herein refers to a construction comprised of genetic material designed to direct transformation of a targeted cell. A vector contains multiple genetic elements positionally and sequentially oriented, i.e., operatively linked with other necessary elements such that the nucleic acid in a nucleic acid cassette can be transcribed and when necessary, translated in the transformed cells. “Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which can be selected from actinomycetes and related species, bacteria and eukaryotic (e.g. higher plant, mammalian, yeast or fungal cells). Preferably the nucleic acid in the vector is under the control of, and operably linked to, an appropriate promoter or other regulatory elements for transcription in a host cell such as a microbial, e.g. bacterial, or plant cell. The vector can be a bi-functional expression vector which functions in multiple hosts. In the case of genomic DNA, this can contain its own promoter or other regulatory elements and in the case of cDNA this can be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector.

The term “plant,” as used herein, refers to any type of plant. The inventors have provided below an exemplary description of some plants that can be used with the invention. However, the list is provided for illustrative purposes only and is not limiting, as other types of plants will be known to those of skill in the art and can be used with the invention.

A common class of plants exploited in agriculture are vegetable crops, including artichokes, kohlrabi, arugula, leeks, asparagus, lettuce (e.g., head, leaf, romaine), bok Choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet and fodder beet), sweet potatoes, swiss-chard, horseradish, tomatoes, kale, turnips, and spices.

Other types of plants frequently finding commercial use include fruit and vine crops such as apples, apricots, cherries, nectarines, peaches, pears, plums, prunes, quince almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pornes, melon, mango, papaya, and lychee.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts), fiber plants (cotton, flax, hemp, jute), Lauraceae (cinnamon, camphor), or plants such as coffee, sugarcane, tea, tomato, potato, cucumber and melon and natural rubber plants.

Another economically important group of plants are ornamental plants. Examples of commonly grown ornamental plants include Alstroemeria (e.g., Alstoemeria brasiliensis), aster, azalea (e.g., Rhododendron sp.), begonias (e.g., Begonia sp.), bellflower, bouganvillea, cactus (e.g., Cactaceae schlumbergera truncata), camellia, carnation (e.g., Dianthus caryophyllus), chrysanthemums (e.g., Chrysanthemum sp.), clematis (e.g., Clematis sp.), cockscomb, columbine, cyclamen (e.g., Cyclamen sp.), daffodils (e.g., Narcissus sp.), false cypress, freesia (e.g., Freesia refracta), geraniums, gerberas, gladiolus (e.g., Gladiolus sp.), holly, hibiscus (e.g., Hibiscus rosasanensis), hydrangea (e.g., Macrophylla hydrangea), juniper, lilies (e.g., Lilium sp.), magnolia, miniroses, orchids (e.g., members of the family Orchidaceae), petunias (e.g., Petunia hybrida), poinsettia (e.g., Euphorbia pulcherima), primroses, rhododendron, roses (e.g., Rosa sp.), snapdragons (e.g., Antirrhinum sp.), shrubs, trees such as forest (broad-leaved trees and evergreens, such as conifers) and tulips (e.g., Tulipa sp.).

The term “plant part”, as used herein, includes reference to, but is not limited to, single cells and tissues from microspores, pollen, ovules, leaves, embryos, roots, root tips, anthers, flowers, fruits, seeds, stems, shoots, scions, rootstocks, protoplasts, calli, meristematic tissues and the like.

The term “adult plant” as used herein refers to the phase of the plant wherein said plant has passed the ontogenic phase as described herein.

A “transgenic plant” is a plant having one or more plant cells that contain an expression vector.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue can be in plants or in organ, tissue or cell culture.

The term “tissue culture”, as used herein, refers to a culture of plant cells wherein the cells are propagated in a nutrient medium under controlled conditions.

The term “perennial”, as used herein, refers to a plant that lives for more than two years. Perennial plants can be short-lived (only a few years) or they can be long-lived, as some woody plants, such as trees.

The term “crop plant”, as used herein, refers to a plant which is harvested or provides a harvestable product. Particularly preferred plants for use in aspects of the invention are row crop plants and/or open field crop plants.

The terms “seedling” and “plantlet”, as used herein, are interchangeable and refer to the juvenile plant grown from a sprout, embryo or a germinating seed and generally include any small plants showing well developed green cotyledons and root elongation and which are propagated prior to transplanting in the ultimate location wherein they are to mature.

“Significant increase” is an increase that is larger than the margin of error inherent in the measurement technique, preferably an increase by about 10%-50%, or even 2-fold or greater.

“Significantly less” means that the decrease is larger than the margin of error inherent in the measurement technique, preferably a decrease by about 2-fold or greater.

The term “early ontogeny”, as used herein, refers to the early phase in the course of growth and development of an individual to maturity (the ontogenic phase). This phase will differ in length between plant species. For some species, this period can amount to about 6 to 8 months post germination, while for others it can be less than a week post germination. In essence, the early ontogenic phase terminates when the plant's characteristics in response to environmental stimuli, such as its respiration rate in particular its AOX, become established and fixed at a certain level. The skilled person can determine the early ontogenic phase for any plant species by measuring the ozone-induced respiration rate (in particular the AOX) in plants at periodic intervals and determining at what age of the plant the increase in respiration rate due to ozone exposure does not return to pre-exposure values, but is fixed. The ontogenic phase for many plants will be the development of the seedling through the vegetative stage and will generally end upon entry of the generative stage (floral stage). As described in the examples below, the fixation of a level of the AOX in many plant species can already be noted after 70 days post germination. The term “early ontogeny” also refers to plants in which tissues are developing, such as plants developing new leave tissues. Preferably the early ontogeny does not relate to fully developed mature tissues such as fully developed mature leaves. The early ontogenic phase can be different for different parts of the plant.

Newly developing leaves can be referred to as being in the early ontogenic phase, whereas maturing and mature leaves are not in the early ontogenic phase. A new meristem that is formed from a callus can be seen as a plant part in an early ontogenic phase according to the present invention. A vegetatively propagated plant material, such as plant cuttings or a tissue culture, will start its early ontogenic phase at the time of root (callus) formation and will end its early ontogenic phase at flowering, or at least at the moment of the first construction (development) of the seeds, or at least at the moment of the filling of the seeds, or at least at the moment after the flowering when the fruits start developing. The early ontogenic phase will generally have ended when 30%, more preferably 40%, 50%, 60%, 70%, 80%, or 90% of the leaves of a plant is mature. Thus, the early ontogenic phase is developmental phase dependent and refers to the early development. In essence, the early ontogenic phase is the phase which ends at the moment where the minimum level of the AOX is no longer sensitive to upscaling by oxidative stress, and the basic level of the minimum level of the AOX is definitively set. This can be determined by measurements as explained in detail herein. The early ontogenic phase is specifically addressed as the phase wherein the ozone protective transgenic system as described herein is most effectively used for fruit protection, seed set protection, flower protection, young plant protection, protection of developing or differentiating tissues, depening on the phase that is agronomically most important for safeguarding the development of the ultimately harvestable product.

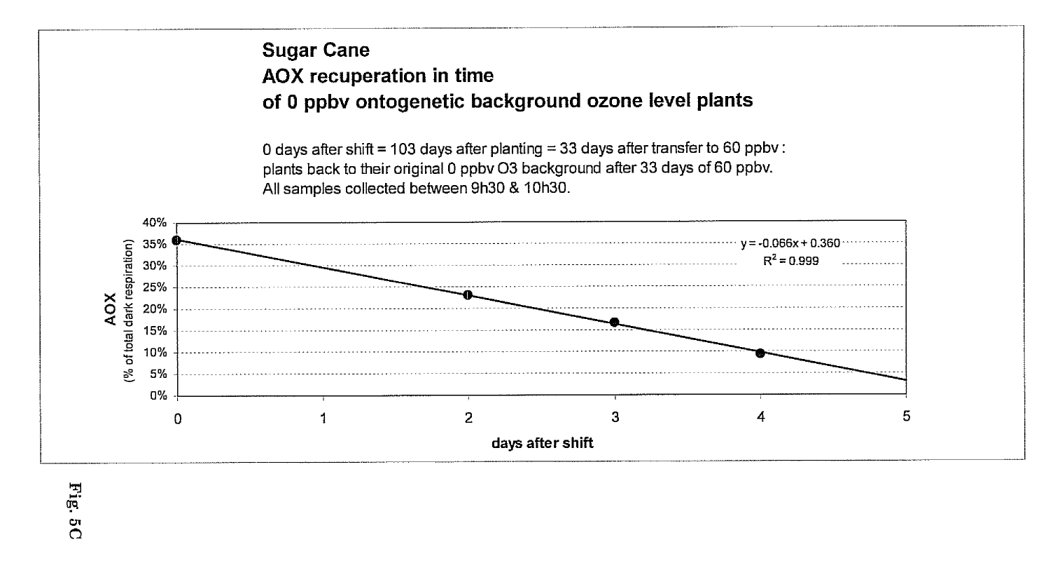

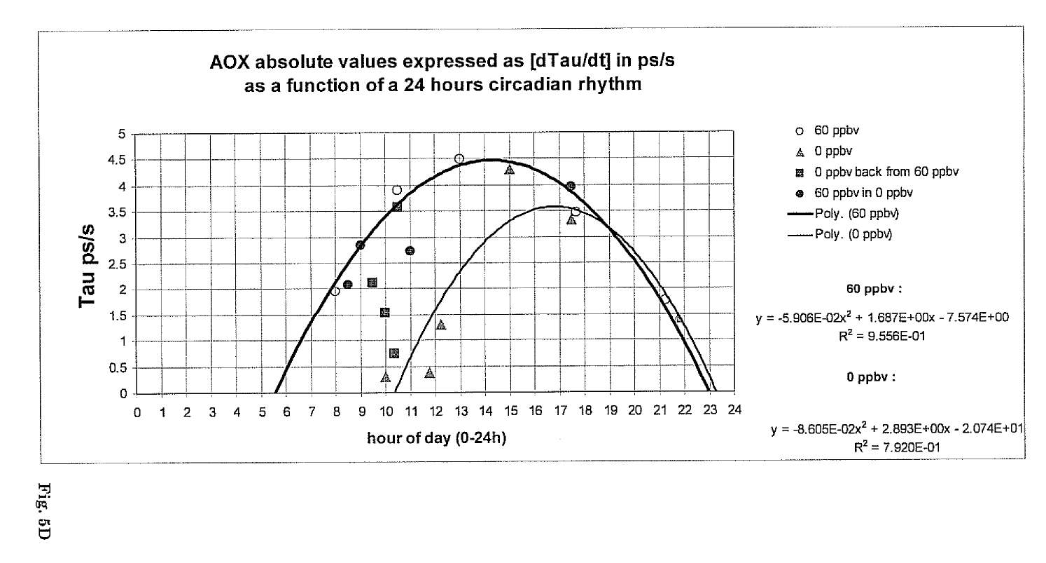

The term “AOX pathway”, as used herein refers to the non ATP rendering Alternative Oxidative Pathway or AOX component within the total dark respiration (TDR). Respiration via this AOX pathway is the “alternative oxidative pathway respiration”, which is also referred to as “alternative path respiration” (APR), “cyanide insensitive respiration” or “SHAM sensitive respiration”. This AOX pathway is activated as shown herein in ambients with higher ozone levels. Although the rate of TDR is usually at constant level for a certain mass of plant material, the rate of respiration via the AOX (shortly referred to herein as “the AOX”) can vary during the day depending on the ambient conditions. Generally, the AOX can be very low during the night. When reference is made to the AOX in general, this may refer to the peak rate of respiration via the AOX (see FIG. 5), or may refer to the low level of the AOX in the absence of any oxidative stress inducing factors.

The term “epigenetic”, as used herein, refers to the state of the DNA with respect to heritable changes in function without a change in the nucleotide sequence. Epigenetic changes can be caused by modification of the DNA, in particular chromatin remodeling caused by modifications of the histone proteins and DNA methylation. These changes affect gene transcription and ultimately affect phenotype. Epigenetic changes thus involve factors that influence behavior of a cell without directly affecting its DNA or other genetic components. The epigenetic view of differentiation is that cells undergo differentiation events that depend on correct temporal and spatial repression, derepression, or activation of genes affecting the fate of cells, tissues, organs, and ultimately, organisms. Thus epigenetic changes in an organism are normal and result in alterations in gene expression. For example, epigenetic transformation of a normal cell to a tumor cell can occur without mutation of any gene.

The term “respiration”, as used herein, refers to the process by which O₂ is transported to and used by the cells and CO₂ is produced and eliminated from the cells during which process organic matter is oxidized.

The term “protein turnover”, as used herein, refers to the flow of amino acids from existing protein into newly synthesized protein. Protein turnover is generally regarded as one of the most important maintenance processes in plants in terms of energy requirements. Both biosynthetic and breakdown processes affect the rate of protein turnover. Both protein synthesis and protein degradation require respiratory energy. Protein turnover has several important functions in regulating the plant's metabolism. Together with protein synthesis, degradation is essential to maintain appropriate enzyme levels and to modulate these levels based on internal and external signals. Furthermore, protein degradation is important in allowing a plant to cope with changing environmental conditions. When nutrients become limiting, the rate of protein turnover is accelerated by increasing the rate of degradation relative to synthesis, which generates a pool of free amino acids from less essential proteins that can be used to assemble more essential ones.

The term “radiation”, as used herein, includes both particle radiation (ie electrons, protons), high-energy electromagnetic radiation (ie x-rays, gamma rays) and other ionizing radiation in the radiant-energy spectrum, as well as non-ionizing electromagnetic radiation, and radiation in the ultraviolet, visible light, and infra-red spectrum.

The term “periodic” in the context of “periodic stress” refers both to a discontinuous form of stress that is experienced by said plant for interrupted periods, as well as to a form of stress that is experienced by said plant for uninterrupted periods (e.g. permanently) but at varying level.

The term “oxidative stress”, as used herein, refers to the state in which cells are exposed to excessive levels of molecular oxygen or reactive oxygen species (ROS) to the extent—ultimately—that damage is incurred and cellular repairs systems are mobilized. Before damage is done however, the plant cells anticipating risk to damage will upscale certain processes (e.g. by protein turnover) and/or down scales other processes (e.g. like cytoplasmic streaming) to prevent damage from occurring. This also qualifies as oxidative stress. Oxidative stress can thus be measured by increased protein turnover rates. As used herein, oxidative stress can inter alia be the result of drought stress, temperature stress, radiation stress, heat stress, salt stress, or reactive oxygen species, for instance such reactive oxygen species as originating from for example epi-phytic or endo-phytic microbiological origin e.g. oxidase positive micro organisms including bacteriae fungi. Oxidative stress as defined herein is therefore the process that increases the AOX pathway. Preventing the plant to increase its AOX pathway during the early ontogeny therefore renders such a plant less sensitive to drought stress, temperature stress, radiation stress, heat stress, salt stress, oxidase positive micro-organisms. Hence methods of the present invention inherently have the result of rendering plants more resistant to other stresses than oxidative stress, including resistance to pests and disease.

The term “reactive oxygen species” (ROS), as used herein, refers to oxygen ions, free radicals, and peroxides, both inorganic and organic. They are generally very small molecules and are highly reactive due to the presence of unpaired valence shell electrons. ROSs form as a natural by-product of the normal metabolism of oxygen and have important roles in cell signalling. However, during times of environmental stress ROS levels can increase dramatically, which can result in significant damage to cell structures. This cumulates into a situation known as oxidative stress.

The term “ambient ozone” is used herein to indicate an ozone level of about 10 to 15 ppbv to about 35 ppbv. The level can vary between night and day and can be about 0 ppbv at night and about 45 to 120 ppbv or higher during the day.

The term “growth environment”, as used herein, refers to the soil, substrate, solution or air in which the plant is growing (also referred to as ambient).

The term “endogenous” as in “endogenously produced” refers to produced within the plant (cell).

The term “anti-oxidant compound” refers to a compound that converts a reactive oxygen species into a non-reactive or harmless form. Suitable examples include, but are not limited to isoprene, terpene, tartaric acid, and ascorbic acid.

The term “isoprene”, as used herein, refers to the chemical compound 2-methylbuta-1,3-diene.

The term “terpene” refers to hydrocarbons derived from isoprene, and includes reference to mono-, di- tri and tetraterpene, sesquiterpene, terpenoid and coenzyme Q, phytol, retinol (vitamin A), tocopherol (vitamin E), dolichol, squalene, heme A, and lanosterol. The term “monoterpene” refers to a terpene that consist of two isoprene units and has the molecular formula C₁₀H₁₆. Monoterpenes can be linear (acyclic) or contain rings. Suitable monoterpenes include α-pinene, β-pinene, sabinene, geraniol, limonene and terpineol.

Terpenoids and terpenes can be synthesized by plants by two metabolic pathways: the mevalonate (MVA) pathway and the methylerythritol phosphate (MEP) pathway.

The MVA pathway (a.k.a. the HMG-CoA reductase pathway) is present in the cytosol of all higher eukaryotes, and plays a role in the production of dimethylallyl pyrophosphate (DMAPP) and its isomer isopentenyl pyrophosphate (IPP), which serve as the basis for the biosynthesis of molecules used in processes as diverse as terpenoid synthesis, protein prenylation, cell membrane maintenance, hormones, protein anchoring, N-glycosylation and steroid biosynthesis.

The MEP pathway (a.k.a. the non-mevalonate pathway), takes place in the plastids of plants. Pyruvate and glyceraldehyde 3-phosphate are converted by DOXP synthase (Dxs) to 1-deoxy-D-xylulose 5-phosphate, and by DOXP reductase (Dxr, IspC) to 2-C-methyl-D-erythritol 4-phosphate (MEP). The subsequent three reaction steps catalyzed by 4-diphosphocytidyl-2-C-methyl-D-erythritol synthase (YgbP, IspD), 4-diphosphocytidyl-2-C-methyl-D-erythritol kinase (YchB, IspE), and 2-C-methyl-D-erythritol 2,4-cyclodiphosphate synthase (YgbB, IspF) mediate the formation of 2-C-methyl-D-erythritol 2,4-cyclopyrophosphate (MEcPP). Finally, MEcPP is converted to (E)-4-hydroxy-3-methyl-but-2-enyl pyrophosphate (HMB-PP) by HMB-PP synthase (GcpE, IspG), and HMB-PP is converted to isopentenyl pyrophosphate (IPP) and dimethylallyl pyrophosphate (DMAPP) by HMB-PP reductase (LytB, IspH).

Thus, IPP and DMAPP are the end-products in either pathway, and are the precursors of isoprene, monoterpenoids (10-carbon), diterpenoids (20-carbon), carotenoids (40-carbon), chlorophylls, and plastoquinone-9 (45-carbon), al of which can be applied as anti-oxidant compounds in aspects of the invention. Synthesis of all higher terpenoids proceeds via formation of geranyl pyrophosphate (GPP), farnesyl pyrophosphate (FPP), and geranylgeranyl pyrophosphate (GGPP). Although both pathways, MVA and MEP, are mutually exclusive in most organisms, interactions between them have been reported in plants. IPP is isomerized to DMAPP by the enzyme isopentenyl pyrophosphate isomerase. Hence, all plants produce IPP and DMAPP and these can be used as precursors for the production of anti-oxidant compounds such as isoprene, by anti-oxidant forming enzymes, such as isoprene synthase.

The term “anti-oxidant enzyme”, refers to an enzyme has anti-oxidant activity in that it or its products convert reactive oxygen species into a non-reactive or harmless (non-damaging) form. Such an enzyme thus effectively neutralizes reactive oxygen species (ROS). Non-limiting examples of anti-oxidant enzymes are glutathione peroxidase (EC 1.11.1.9), glutathione reductase (EC 1.8.1.7, formerly EC 1.6.4.2), catalase (EC 1.11.1.6), thioredoxin reductase (EC 1.8.1.9), superoxide dismutase (SOD, EC 1.15.1.1), heme oxygenase (EC 1.14.99.3) and biliverdin reductase (EC 1.3.1.24). The term “enzyme”, as used throughout this specification, should not be construed as being limited to biocatalytic proteins, but also to other proteins and polypeptides such as for instance the anti-oxidant protein from Phyllanthus niruri as disclosed in Sarkar et al 2009 Food Chemistry 114 (4):1405-1412.

The term “an enzyme that produces an antioxidant compound” refers to an enzyme that, when expressed in a plant cell, result in the production of an anti-oxidant compound, and includes reference to such enzymes as isoprene synthase, glutathione reductase, dehydroascorbate reductase (EC 1.8.5.1), L-galactono-γ-lactone dehydrogenase (or L-galactono-1,4-lactone dehydrogenase, EC 1.3.2.3), phosphomannomutase (PPM, EC 2.7.5.7), GDP-D-mannose pyrophosphorylase (GMP, EC 2.7.7.22), GDP-mannose-3′,5′-epimerase (EC 5.1.3.18), Gal-UR (NADPH-dependent D-galacturonate reductase, EC 1.1.1.203), miox4 (the myo-inositol oxygenase, EC 1.13.99.1), L-idonate dehydrogenase (EC 1.1.1.128) and other enzymes of the ascorbate-glutathione cycle in higher plants. All such enzymes and homologues thereof, and in particular the genes encoding them, are in principle suitable for use in aspects of the present invention. Isoprene synthase is highly preferred embodiment of an enzyme that produces an antioxidant compound in aspects of the present invention.

The term “isoprene synthase”, as used herein, refers to the enzyme with registry number EC 4.2.3.27, that catalyzes the elimination of pyrophosphate from dimethylallyl diphosphate to form isoprene. The amino acid sequence of the enzyme in Populus alba is provided in SEQ ID NO:1. The term is considered to cover homologues having >80%, preferably >90% amino acid identity with SEQ ID NO:1, and in particular refers to homologues that produce isoprene from dimethylallyl diphosphate. SEQ ID No: 2 provides the nucleotide sequence for the gene encoding the isoprene synthase enzyme as set forth in Genbank accession EF638224.

The term “prefloral stage”, as used herein, refers to the ontogenic stage preceding the emergence of reproductive structures in a plant.

The term “post-germination”, as used herein, refers to the period in the development of the plant following the emergence of the radicle from the seed.

The term “average yearly crop production”, as used herein, refers to the amount of crop produced per annum or season (average seasonal crop production) in the form of weights or number of plants or plant parts harvested or weight gain in crop biomass (fresh and or dry weight) are expressed per unit of production area, and wherein the individual amounts per annum for multiple years are summated and divided by the number of years.

The term “production area”, as used herein, refers to a location where plants are grown and where products in the form of plants or plant parts are produced for harvest. The size of the production area is generally expressed in square meters or acres of land. A production area can be an open field or a greenhouse.

The term “net carbon fixation efficiency”, as used herein, is used interchangeable with the term “net photosynthetic efficiency” and refers to the net efficiency with which carbon dioxide is converted into organic compounds, taking into account the losses due to respiration.

The term “biomass production”, as used herein, refers to the production of plant derived organic material.

The term “dry matter content”, as used herein, refers to the mass fraction (%) that remains after the water fraction (%) has been removed by drying.

The term “pest resistance”, as used herein, refers to resistance against viral, bacterial, fungal, and insect pests, as well as pests by molluscs and nematodes.

### DNA Sequences for Transformation

Virtually any DNA composition can be used for delivery to recipient plant cells, to ultimately produce fertile transgenic plants in accordance with the present invention. For example, DNA segments in the form of vectors and plasmids, or linear DNA fragments, in some instances containing only the DNA element to be expressed in the plant, and the like, can be employed. The construction of vectors which can be employed in conjunction with the present invention will be known to those of skill of the art in light of the present disclosure (see, e.g., Sambrook et al., 1989; Gelvin et al., 1990). Vectors, including plasmids, cosmids, YACs (yeast artificial chromosomes), BACs (bacterial artificial chromosomes) and DNA segments for use in transforming cells, according to the present invention will, of course, comprise the cDNA, gene or genes necessary for production of the anti-oxidant enzyme of the enzyme that produces anti-oxidant compounds, such as isoprene, in the transformant.

The vector of the invention can be introduced into any plant. The genes and sequences to be introduced can be conveniently used in expression cassettes for introduction and expression in any plant of interest. Such expression cassettes will comprise a transcriptional initiation region (a promoter) linked to the gene encoding the (heterologous) gene of interest. Such an expression cassette is preferably provided with a plurality of restriction sites for insertion of the gene of interest to be under the transcriptional regulation of the regulatory regions, such as the designated promoter. The expression cassette can additionally contain selectable marker genes suitable for the particular host organism.

The transcriptional cassette will include in the 5′-to-3′ direction of transcription, transcriptional and translational initiation regions, a DNA sequence of interest, and transcriptional and translational termination regions functional in plants.

The termination region can be native with the transcriptional initiation region, can be native with the DNA sequence of interest, or can be derived from another source.

Convenient termination regions are available from the Ti-plasmid of A. tumefaciens, such as the octopine synthase and nopaline synthase termination regions. See also, Guerineau et al. (1991) Mol. Gen. Genet. 262: 141-144; Proudfoot (1991) Cell 64: 671-674; Sanfacon et al. (1991) Genes Dev. 5: 141-149; Mogen et al. (1990) Plant Cell 2: 1261-1272; Munroe et al. (1990) Gene 91: 151-158; Ballas et al. (1989) Nucleic Acids Res. 17: 7891-7903; Joshi et al. (1987) Nucleic Acid Res. 15: 9627-9639.

Methodologies for the construction of plant transformation constructs are described in the art.

Overexpression can be achieved by insertion of one or more than one extra copy of the selected gene. It is not unknown for plants or their progeny, originally transformed with one or more than one extra copy of a nucleotide sequence to exhibit overexpression.

Obtaining sufficient levels of transgene expression in the appropriate plant tissues is an important aspect in the production of genetically engineered crops. Expression of (heterologous) DNA sequences in a plant host is dependent upon the presence of an operably linked promoter that is functional within the plant host. Choice of the promoter sequence will determine when and where within the organism the (heterologous) DNA sequence is expressed.

Preferred genes to be expressed in the present invention are the isoprene synthase gene, resulting in the production of isoprene, genes that cause an enhanced presence of ascorbic acid and genes that cause an enhanced presence of tartaric acid.

Ascorbic acid has been identified as one of the major oxidants in plants, protecting them from oxidative stress, such as caused by ozone. This function is exerted mainly by the ascorbate that is present in the cell wall. Also, ascorbate is found in the chloroplasts, indicating that it also plays a role in photosynthesis (in the scavenging of hydrogen peroxide and in the formation of the photoprotectant zeaxanthin). Foyer, C. H. et al. (2001, Plant Physiol. 97:863-872) have shown that (over)expression of glutathione reductase, which converts monodehydro-L-ascorbic acid to ascorbate, increases resistance to oxidative stress. This has been confirmed by Chen, Z. et al. (2003, Proc. Natl. Acad. Sci. USA, 100:3525-3530) who used DHAR (dehydro-ascorbate reductase).

Also, plants overexpressing the enzyme L-galactono-gamma-lactone dehydrogenase have an increased ascorbate content and thus an increased resistance to oxidative stress (EC 1.3.2.3; GLDase) (Oestergaard, J. et al., 1997, J. Biol. Chem., 272:30009-16; U.S. Pat. No. 6,469,149). Recently, several other enzymes have been reported or suggested to increase the ascorbic acid content in plants when provided transgenically: Phosphomannomutase (PMM; EC 5.4.2.8) (Badejo, A. A. et al., 2009, Plant Cell Physiol. 19122187), GDP-D-mannose pyrophosphorylase (GMP) (Badejo, A. A. et al., 2007, Plant Cell Physiol. 18037674), GDP-mannose-3′,5′-epimerase and L-galactono-1,4-lactone dehydrogenase (Valpuesta, V. and Botella, M. A., 2004, Trends Plant Sci. 9(12):573-577); Gal-UR, an NADPH-dependent D-galacturonate reductase (Agius, F. et al., 2003, Nature Biotechnol. 21:177-181), and miox4, a myo-inositol oxygenase (WO 2004/061098).

Tartaric acid (TA) is a white crystalline organic acid, and is abundant in grapes. It is one of the main acids found in wine and is added as a flavouring in foods and beverages, and is used as an antioxidant. Tartaric acid is practically resistant to microbial conversion in grape juice or wine and hence often chosen as the acid for addition to reduce grape juice and wine pH values. Furthermore, tartaric acid equilibria are important in wine production; for example, to control acidity, pH and tartrate stability. Elucidation of the intermediates relating L-tartaric acid to vitamin C catabolism showed that they proceed via L-idonic acid, the proposed rate-limiting step in the pathway. For overexpression of TA a novel enzyme, having L-idonate dehydrogenase activity, has been demonstrated (WO 2007/006087).

### Transcription Regulatory Sequences

### Promoters

The above mentioned genes in transgenic plants of the invention are under the control of a promoter. Suitable promoters are regulated promoters which include, but are not limited to, inducible, temporally regulated, developmentally regulated, spatially-regulated, chemically regulated, stress-responsive, and tissue-specific promoters and combinations thereof.

Examples thereof have inter alia been indicated above. An inducible promoter is a promoter that provides for the turning on and off of gene expression in response to an exogenously added agent, such as a chemical agent or biological agent (eg. a herbivore or pathogen) or to an environmental or developmental stimulus.

A range of naturally-occurring promoters are known to be operative in plants and have been used to drive the expression of (heterologous) genes in plants: for example, the gamma zein promoter, the oleosin ole16 promoter, the globulin promoter, the actin I promoter, the actin cl promoter, the sucrose synthetase promoter, the INOPS promoter, the EXM5 promoter, the globulin2 promoter, the b-32, ADPG-pyrophosphorylase promoter, the LtpI promoter, the Ltp2 promoter, the oleosin ole17 promoter, the oleosin old 8 promoter, the actin 2 promoter, the pollen-specific protein promoter, the pollen-specific pectate lyase promoter, the anther-specific protein promoter, the anther-specific gene RTS2 promoter, the pollen-specific gene promoter, the tapeturn-specific gene promoter, the tapeturn-specific gene RAB24 promoter, the anthranilate synthase alpha subunit promoter, the alpha zein promoter, the anthranilate synthase beta subunit promoter, the dihydrodipicolinate synthase promoter, the Thil promoter, the alcohol dehydrogenase promoter, the cab binding protein promoter, the H3C4 promoter, the RUBISCO SS starch branching enzyme promoter, the ACCase promoter, the actin3 promoter, the actin7 promoter, the regulatory protein GF14-12 promoter, the ribosomal protein L9 promoter, the cellulose biosynthetic enzyme promoter, the S-adenosyl-L-homocysteine hydrolase promoter, the superoxide dismutase promoter, the C-kinase receptor promoter, the phosphoglycerate mutase promoter, the root-specific RCc3 mRNA promoter, the glucose-6 phosphate isomerase promoter, the pyrophosphate-fructose 6-phosphatelphosphotransferase promoter, the ubiquitin promoter, the beta-ketoacyl-ACP synthase promoter, the 33 kDa photosystem II promoter, the oxygen evolving protein promoter, the 69 kDa vacuolar ATPase subunit promoter, the metallothionein-like protein promoter, the glyceraldehyde-3-phosphate dehydrogenase promoter, the ABA- and ripening-inducible-like protein promoter, the phenylalanine ammonia lyase promoter, the adenosine triphosphatase S-adenosyl-L-homocysteine hydrolase promoter, the a-tubulin promoter, the cab promoter, the PEPCase promoter, the R gene promoter, the lectin promoter, the light harvesting complex promoter the a heat shock protein promoter, the chalcone synthase promoter, the zein promoter, the globulin-1 promoter, the ABA promoter, the auxin-binding protein promoter, the UDP glucose flavonoid glycosyl-transferase gene promoter, the NTI promoter, the actin promoter, the opaque 2 promoter, the b70 promoter, the oleosin promoter, the CaMV 35S promoter, the CaMV 19S promoter, the histone promoter, the turgor-inducible promoter, the pea small subunit RuBP carboxylase promoter, the Ti plasmid mannopine synthase promoter, the Ti plasmid nopaline synthase promoter, the petunia chalcone isomerase promoter, the bean glycine rich protein I promoter, the CaMV 35S transcript promoter, the potato patatin promoter, or the S-E9 small subunit RuBP carboxylase promoter. Many of these promoters are suitable for use in aspects of the invention provided their expression is or can be regulated, and is not constitutive and/or does not occur in every part of said plant. Preferably the promoter drives gene expression that allows the said plant to neutralize ROS during the ontogenic phase as described herein. Preferably, the promoter does not drive expression in said plant when said plant has passed the ontogenic phase, and the promoter activity is preferably lost such that the expression of the gene is significantly decreased or stopped to the extent that unnecessary carbon loss due to said expression is prevented.

Inducible Promoters (“gene switches”)

Several inducible promoters (“gene switches”) have been reported. Many are described in the review by Gatz (1996) and Gatz (1997). These include tetracycline repressor system, Lac repressor system, copper-inducible systems, salicylate-inducible systems (such as the PR1a system), glucocorticoid—(Aoyama et al., 1997) and ecdysone-inducible systems. Also included are the benzene sulphonamide—(U.S. Pat. No. 5,364,780) and alcohol—(WO 97/06269 and WO 97/06268) inducible systems and glutathione S-transferase promoters. Other suitable inducible promoters include promoters that are inducible by ozone, NOx, and other (oxidative) stresses. Other studies have focused on genes inducibly regulated in response to environmental stress or stimuli such as increased salinity, drought, pathogen and wounding (Graham et al., 1985; Graham et al., 1985, Smith et al., 1986). Accumulation of metallocarboxypeptidase-inhibitor protein has been reported in leaves of wounded potato plants (Graham et al., 1981). Other plant genes have been reported to be induced methyl jasmonate, elicitors, heat-shock, anaerobic stress, or herbicide safeners, the promoters of such genes can be used in combination with the specific stimulus to achieve inducible expression of the gene.

The use of promoters that are chemically regulated allows for the genes to be expressed only when the plants are treated with the inducing chemicals. Chemical induction of gene expression is detailed in EP 0 332 104 and U.S. Pat. No. 5,614,395. A preferred promoter for chemical induction is the tobacco PR-1a promoter. Other examples of inducible promoters include ABA- and turgor-inducible promoters and the promoter of the auxin-binding protein gene (Schwob et al., Plant J. 4(3):423-32 (1993); Genbank Accession No. L08425). Still other potentially useful promoters include the UDP glucose flavonoid glycosyltransferase gene promoter (Ralston et al., 1988. Genet., 119(1):185-197); MPI proteinase inhibitor (Cordero et al., 1994. Plant J, 6(2):141-150), and the glyceraldehyde-3-phosphate dehydrogenase gene promoter (Kohler et al., 1995. Plant Mol. Biol., 29 (6):1293-1298; Quigley et al., 1989. J. Mol. Evol., 29 (5):412-421), as well as promoters of chloroplast genes (Genbank Accession No. X86563). U2 and U5 snRNA promoters from maize, the inducible promoter from alcohol dehydrogenase, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD-zein protein, inducible promoters, such as the light inducible promoter derived from the pea rbcS gene and the actin promoter from rice, e.g., the actin 2 promoter (WO 00/70067), can also be used.

Inducible promoters can be induced by using any agent (chemical or biological) capable of inducing the activation of the promoter. Promoter-inducing agents can be used in combination with gene-silencing agents, to control gene expression temporally.

### Developmentally Regulated or Developmental Phase-Specific Promoters

To obtain expression of a transgene specifically in the ontogenetic phase, it is also possible to combine a coding sequence with an inducible promoter or to put it under control of a gene switch system. In the description above, examples of these promoters and systems have been given.

It will be easily achievable for a person skilled in the art to activate the inducible promoter during the ontogenic phase of the plant by application of the specific compound that will induce or activate the promoter. E.g. in the example of the AlcA/AlcR switch system, application of alcohol, whether as liquid spray or as vapour in the air, will be sufficient to activate expression of the gene that is under control of the AlcA/AlcR promoters. Of course, this will be very advantageous when the plants are grown under controlled climate conditions, in which administration of the inducer compound to the plants is fairly easy. But also under field conditions, such application is feasible, e.g. by spraying from the air.

In one highly preferred embodiment of transgenic plants of the present invention, the above mentioned genes are mainly expressed during the ontogenetic phase. For this purpose promoters should be chosen which are specifically active during said ontogenetic phase.

Promoters that are specifically active during the ontogenetic phase are the Pyk10 promoter from Arabidopsis thaliana, which has the sequence of SEQ ID NO:3 (GenBank: AJ292756.1) or the promoter parts of this sequence that confer the ontogenetic phase-specific activity of this promoter. Further useful promoters are the malate synthase and isocitrate lyase plant promoter sequences as described in U.S. Pat. No. 5,689,040, which is herein incorporated by reference. This document shows the specific promoter sequences as give below (and useful parts thereon of the malate synthase (SEQ ID NO:4) and isocitrate lyase (SEQ ID NO:5) genes from Brassica napus and the ways how to use these promoters in transcription vectors.

A further promoter that can be used as an ontogenetic phase specific promoter is the promoter of the gene encoding the protein GS-box binding factor 1 (GSBF1). It has been established that transgenic GSBF1 transcripts are cotyledon-specific and accumulate to the highest levels during late seedling development in a light-dependent manner (Waldmüller, S. et al., 1996, Plant Mol. Biol. 32:631-639).

Further applicable, especially for use in monocot plants, is the promoter of a rice glycine-rich RNA binding protein gene as described in U.S. Pat. No. 6,376,750, of which the minimal promoter sequence is given in SEQ ID NO:6, while the full promoter has the sequence of SEQ ID NO:7. Also parts of this promoter are especially applicable (e.g. the part from −500 to the transcription start site 0).

Tissue specific enhancers from the octopine synthase gene that confer expression in seedlings are described in Fromm et al. 1989 (The Plant Cell, 1:977-984).

Other examples of seedling-specific promoters that can be used in the present invention are the oil seed rape cysteine protease gene promoter as described in U.S. Pat. No. 6,228,643 and the promoters of the two lipid transfer protein genes from barley that have been described by Gausing, K (1994, Planta, 192:574-580). The person skilled in the art will be able to find alternative promoters for the ones that have been mentioned herein.

### Tissue-Specific Promoters

Further, next to specific expression in relation to the developmental phase of the plant, in many cases also tissue-specific expression is preferred, most preferably leaf-specific expression. The ROS neutralizing genes in transgenic plants of the present invention are in certain embodiments preferably expressed in a tissue-specific manner. For the specific expression of the genes that are useful in the present invention, it will depend on the characteristics of the gene and/or the availability of the substrates for the gene product and/or the tissue in which the gene product exerts its intended function, in which tissue(s) the gene should preferentially be expressed. While in some cases, expression in multiple tissues is desirable, it is in this embodiment preferred that the expression in transgenic plants of the present invention is leaf-specific.

To obtain tissue specific promoters, genes encoding tissue specific messenger RNA (mRNA) can be obtained by differential screening of a cDNA library. For example, a leaf-preferred cDNA can be obtained by subjecting a leaf cDNA library to differential screening using cDNA probes obtained from leaf and seed mRNA. See, Molecular Cloning, A Laboratory Manual, Sambrook et al. eds. Cold Spring Harbor Press: New York (1989).

Alternately, leaf specific promoters can be obtained by obtaining leaf specific proteins, sequencing the N-terminus, synthesizing oligonucleotide probes and using the probes to screen a cDNA library. Such procedures are well known in the art.

In highly preferred embodiments, the expression of the gene occurs in the leaf plastids. The promoter of the isoprene synthase gene from Populus alba (PaIspS) (Sasaki et al. 2005 FEBS Letters 579(11):2514-2518) can drive plastid-specific expression. Hence, this promoter is a very suitable promoter for use in an expression vector of the present invention.

Numerous promoters whose expression are known to vary in a tissue specific manner are known in the art. One such example is the maize phosphoenol pyruvate carboxylase (PEPC), which is green tissue-specific. See, for example, Hudspeth, R. L. and Grula, J. W., Plant Molecular Biology 12:579-589, 1989). Other green tissue-specific promoters include chlorophyll a/b binding protein promoters and RubisCO small subunit promoters. Other suitable leaf-specific promoters are the rbcS (Rubisco) promoter (e.g. from coffee, see WO 02/092822); from Brassica, see U.S. Pat. No. 7,115,733; from Chrysanthemum, see Outchkourov et al. (2002); from Phaseolus vulgaris (see Hiratsuka and Chua (1997); from soybean, see Dhanker, O., et al., 2002, Nature Biotechnol. 20:1140-1145), the cy-FBPase promoter (see U.S. Pat. No. 6,229,067), the promoter sequence of the light-harvesting chlorophyll a/b binding protein from oil-palm (see US 2006/0288409), the STP3 promoter from Arabidopsis thaliana (see, Buttner, M. et al., 2001, Plant cell & Environ. 23:175-184), the promoter of the bean PAL2 gene (see Sablowski, R. W. et al., 1995, Proc. Natl. Acad. Sci. USA 92:6901-6905), enhancer sequences of the potato ST-LS1 promoter (see Stockhaus, J. et al., 1985, Proc. Natl. Acad. Sci. USA 84:7943-7947), the wheat CAB1 promoter (see Gotor, C. et al., 1993, Plant J. 3:509-518), the stomata-specific promoter from the potato ADP-glucose-phosphorylase gene (see U.S. Pat. No. 5,538,879), the LPSE1 element from the P(D540) gene of rice (see CN 2007/10051443), and the stomata specific promoter, pGC1(At1g22690) from Arabidopsis thaliana (see Yang, Y. et al., 2008, Plant Methods 4:6). This list is only exemplary, and the person skilled in the art will be able to find many more promoters or promoter-elements that have been demonstrated to confer specificity for expression in leaf-tissue.

For genes that are involved in the biochemical pathways for the production of ascorbic acid, leaf-specific and/or stomata-specific expression is preferred. Leaf specific promoters are well known in the art and several have already been mentioned above. Most preferred, however, are promoters that specifically cause expression in the stomatal cells (guard cells).

The tissue-specificity of some “tissue-specific” promoters may not be absolute and can be tested by one skilled in the art using the diphtheria toxin sequence. One can also achieve tissue-specific expression with “leaky” expression by a combination of different tissue-specific promoters (Beals et al., 1997). Other tissue-specific promoters can be isolated by one skilled in the art (see U.S. Pat. No. 5,589,379).

Vectors for use in tissue-specific targeting of genes in transgenic plants will typically include tissue-specific promoters and can also include other tissue-specific control elements such as enhancer sequences.

Additionally, vectors can be constructed and employed in the intracellular targeting of a specific gene product within the cells of a transgenic plant. This will generally be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane.

A particular example of such a use concerns the direction of a protein (enzyme) to a particular organelle such as the chloroplast rather than to the cytoplasm. This is exemplified by the use of the rbcs transit peptide which confers plastid-specific targeting of proteins. By facilitating the transport of the protein into compartments inside the cell, these transit peptides can increase the accumulation of gene product by protection from proteolytic degradation.

Other tissue-specific promoters that are envisioned in preferred embodiments of the invention include stomata-specific promoters. Use of such promoters for instance enables expression of the ROS neutralizing genes specifically in the stomatal guard cells. Preferably there is expression in the stomatal guard cells and no expression in the mesophyllic or epidermal cells of the plant leafs or plant stem, but there may be expression in parts of the root or the flowers. Such specific overexpression can be realized by the endogenous Arabidopsis thaliana TRE1 promoter (WO2010/089392), the stomata-specific Arabidopsis thaliana pGC1(At1g22690) promoter, the potato KST1 promoter (Plesch et al., Plant J. 28(4):455-64 (2001)), the Rhal promoter (Terryn et al., 1993, Plant Cell 5:1761-1769), and the Arabidopsis stomatal-specific promoter sequences of the AtMYB60 gene (At1g08810; GenBank acc. no. AF062895; U.S. Pat. No. 7,662,947), or to a fragment of one of these promoters having promoter activity. The skilled person is aware that different regions of AtMYB60 promoter enable either ABA-responsive or ABA-independent selective expression of nucleic acids in stomatal guard cells. The full length or a fragment of the full-length AtMYB60 promoter sequence (SEQ ID No. 1 in U.S. Pat. No. 7,662,947) may be used such as SEQ ID No. 2 in U.S. Pat. No. 7,662,947 (from nucleotide (nt) 1045 to 1291), SEQ ID No. 3 in U.S. Pat. No. 7,662,947 (nt 689-1291) or SEQ ID No. 4 in U.S. Pat. No. 7,662,947 (nt 293-1292 of SEQ ID No. 1 therein). The fragment extending from nt 1045 to nt 1291 of SEQ ID NO. 1 in U.S. Pat. No. 7,662,947 exhibits an ABA-independent promoter activity. Whereas the activity of the fragments containing SEQ ID No. 3 and 4 in U.S. Pat. No. 7,662,947, as well as the activity of the full-length promoter (SEQ ID No. 1 in U.S. Pat. No. 7,662,947), are down-regulated by abscisic acid. Hence, the skilled person will appreciate that the stoma-specific expression of the ROS neutralizing genes can be modulated in either ABA-dependent or ABA-independent manner using different gene constructs or expression cassettes.

### General Considerations

In general, in transgene expression it is advantageous to only express the (heterologous) nucleic acid at times and in places where the expression product can exert an effect. If this is not the case, precious cell machinery is occupied with the production of ‘useless’ proteins. This energy costing production of proteins then will negatively influence the other processes in the plant that are e.g. needed for photosynthesis and growth, transport of water and other compounds, and defence. It is thus submitted that plants that produce proteins in places and on times that are nut fully necessary are less fit than plants that only transcribe a gene at a moment where it will be advantageous. Therefore, any expression of transgenes in a period in the life of the plant wherein the gene products are not able to fulfil their desired function are preferably minimized.

Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

Use can be made in aspects of the present invention of full length promoters, or of minimal promoter regions. Thus, the scope of the present invention in regard to the regulated promoters encompasses functionally active fragments of a full-length promoter that also are able to direct the regulated transcription, respectively, of associated structural genes. Functionally active fragments of a promoter DNA sequence can be derived from a promoter DNA sequence, by several art-recognized procedures, such as, for example, by cleaving the promoter DNA sequence using restriction enzymes, synthesizing in accordance with the sequence of the promoter DNA sequence, or can be obtained through the use of PCR technology. See, e.g. Mullis et al., Meth. Enzymol. 155:335-350 (1987); Erlich (ed.), PCR Technology, Stockton Press (New York 1989).

Further included within the scope of the instant invention are regulated promoters “equivalent” to the full-length promoters. That is, different nucleotides, or groups of nucleotides can be modified, added or deleted in a manner that does not abolish promoter activity in accordance with known procedures.

To define a minimal promoter region, a DNA segment representing the promoter region is removed from the 5′ region of the gene of interest and operably linked to the coding sequence of a marker (reporter) gene by recombinant DNA techniques well known to the art. For this, a reporter gene can be operably linked downstream of the promoter, so that transcripts initiating at the promoter proceed through the reporter gene. Reporter genes generally encode proteins which are easily measured, including, but not limited to, chloramphenicol acetyl transferase (CAT), beta-glucuronidase (GUS), green fluorescent protein (GFP), beta-galactosidase (beta-GAL), and luciferase. The construct containing the reporter gene under the control of the promoter(s) is then introduced into an appropriate cell type by transfection techniques well known to the art. To assay for the reporter protein, cell lysates are prepared and appropriate assays, which are well known in the art, for the reporter protein are performed. For example, if CAT were the reporter gene of choice, the lysates from cells transfected with constructs containing CAT under the control of a promoter under study are mixed with isotopically labeled chloramphenicol and acetyl-coenzyme A (acetyl-CoA). The CAT enzyme transfers the acetyl group from acetyl-CoA to the 2- or 3-position of chloramphenicol. The reaction is monitored by thin-layer chromatography, which separates acetylated chloramphenicol from unreacted material. The reaction products are then visualized by autoradiography. The level of enzyme activity corresponds to the amount of enzyme that was made, which in turn reveals the level of expression from the promoter of interest. This level of expression can be compared to other promoters to determine the relative strength of the promoter under study. In order to be sure that the level of expression is determined by the promoter, rather than by the stability of the mRNA, the level of the reporter mRNA can be measured directly, such as by Northern blot analysis. Once activity is detected, mutational and/or deletional analyses can be employed to determine the minimal region and/or sequences required to initiate transcription. Thus, sequences can be deleted at the 5′ end of the promoter region and/or at the 3′ end of the promoter region, and nucleotide substitutions introduced. These constructs are then introduced to cells and their activity determined.

### Production and Characterization of Stably Transformed Plants

Plants can be transformed by any suitable transformation technique, such as by DNA-mediated transformation of plant cell protoplasts and subsequent regeneration of the plant from the transformed protoplasts in accordance with procedures well known in the art.

As an example, the following procedure as described by Sharkey et al. 2005 (Plant Physiol. 137(2): 700-712) can be adapted to suit the plant species to be transformed, using suitable promoters and ant-oxidant encoding genes as described herein. A suitable isoprene synthase genomic sequence can be amplified using specific oligonucleotides (for instance for Kudzu, sense primer, 5′-GTGTGCCCGGGTGGTTGAGTTGGTCCATTGAAGTA-3′; and antisense primer, 5′-ACACACCCGGGGATTTGATGCCTTTCCTGATTTTA-3′ can be used) that can be flanked with SmaI restriction sites (underlined). For Kudzu, this will result in the amplification of a fragment including 270 bp upstream of the ATG translational start site and ending near the translational stop codon. The amplified DNA can then be cloned into the pGEM-T vector (Promega, Madison, Wis.). The insert can be released with the SmaI restriction enzyme and cloned into the pPZP212 Agrobacterium tumefaciens binary vector (Hajdukiewicz et al., 1994 Plant Mol Biol 25: 989-994) at the SmaI site to produce pGISPS. The pGISPS plasmid can then be transformed into a suitable A. tumefaciens strain, such as strain GV3101.

The target plant can then be transformed using any suitable method for the selected target plant, for instance the whole-plant floral dipping method for A. thaliana (Clough and Bent, 1998 Plant J 16: 735-743) can be used. Transformed individuals (T1 generation) can then be selected on a suitable medium, such as the germination medium, supplemented with 75 μg/mL kanamycin before transfer to soil. The number of unlinked T-DNA loci within each line can be determined by analyzing the segregation of kanamycin resistance in the progeny (T2 generation).

The rate of isoprene emission from transformed plants can be determined by enclosing plants in a chamber and sampling the headspace after for instance 10 min under inducing conditions (conditions that induce isoprene synthase expression and isoprene formation). Additionally, detached leaves can be enclosed in small vials for 30 to 60 min followed by sampling of the head space gas for isoprene analysis. Isoprene concentrations in the sampled head space can be determined by mass spectrometry. For such analyses air samples (0.01-31) can be taken and condensed by adsorption using MX-06-2131 sorbent tubes (CDS Analytical, Inc, Oxford, Pa., USA). Such tubes are packed with 20:35 Tenax-TA™/60:80 Carboxen™ 1000/60:80 Carbosieve™ SIII as the adsorbent material.

Isoprene synthase activity can be measured for instance in a pH 8.0 buffer (50 mM bicine buffer, 50 mM MgCl₂, 5 mM KCl, 2 mM NaF, 5% glycerol and 5 mm dithiothreitol). Reactions can be carried out in small (eg. 5.5-mL) sealed vials at 35° C. After 15 min, 3 mL of the head space gas can be removed by syringe and can be injected on a gas chromatograph with photoionization detection for quantification. Reactions can be typically run with 12 mm DMAPP and DMAPP can be synthesized as described by Davisson et al. 1985 (Methods Enzymol 110: 130-144).

Any plant tissue capable of subsequent clonal propagation, whether by organogenesis or embryogenesis, can be transformed with a vector of the present invention. The term “organogenesis”, as used herein, means a process by which shoots and roots are developed sequentially from meristematic centers; the term “embryogenesis”, as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants of the present invention can take a variety of forms. The plants can be chimeras of transformed cells and non-transformed cells; the plants can be clonal transformants (e.g., all cells transformed to contain the expression cassette); the plants can comprise grafts of transformed and untransformed tissues (e.g., a transformed root stock grafted to an untransformed scion in citrus species). The transformed plants can be propagated by a variety of means, such as by clonal propagation or classical breeding techniques. For example, first generation (or T1) transformed plants can be selfed to give homozygous second generation (or T2) transformed plants, and the T2 plants further propagated through classical breeding techniques. A dominant selectable marker (such as npt II) can be associated with the expression cassette to assist in breeding.

Thus, the present invention provides a transformed (transgenic) plant cell, in planta or ex planta, including a transformed plastid or other organelle, e.g., nucleus, mitochondria or chloroplast.

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

Suitable methods of transforming plant cells include, but are not limited to, microinjection (Crossway et al., 1986), electroporation (Riggs et al., 1986), Agrobacterium-mediated transformation (Hinchee et al., 1988), direct gene transfer (Paszkowski et al., 1984), and ballistic particle acceleration using devices available from Agracetus, Inc., Madison, Wis. And BioRad, Hercules, Calif. (see, for example, Sanford et al., U.S. Pat. No. 4,945,050; and McCabe et al., 1988). Also see, Weissinger et al., 1988; Sanford et al., 1987 (onion); Christou et al., 1988 (soybean); McCabe et al., 1988 (soybean); Datta et al., 1990 (rice); Klein et al., 1988 (maize); Klein et al., 1988 (maize); Klein et al., 1988 (maize); Fromm et al., 1990 (maize); and Gordon-Kamm et al., 1990 (maize); Svab et al., 1990 (tobacco chloroplast); Koziel et al., 1993 (maize); Shimamoto et al., 1989 (rice); Christou et al., 1991 (rice); European Patent Application EP 0 332 581 (orchardgrass and other Pooideae); Vasil et al., 1993 (wheat); Weeks et al., 1993 (wheat). In one embodiment, the protoplast transformation method for maize is employed (European Patent Application EP 0 292 435, U.S. Pat. No. 5,350,689).

It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al., 1985 Bio/Technology 3: 241; Sukhapinda et al., 1987 Plant Mol. Biol. 8, pp. 209-216; Park et al., 1995 J. Plant Biol. 38, pp. 365-371; Hiei et al., 1994 Plant J. 6, pp. 271-282). The use of T-DNA to transform plant cells has received extensive study and is amply described (EP 0 120 516; and An et al., 1985 EMBO J. 4, pp. 277-284). For introduction into plants, the chimeric genes of the invention can be inserted into binary vectors.

Other transformation methods are available to those skilled in the art, such as direct uptake of foreign DNA constructs (see EP 0 295 959), techniques of electroporation (Fromm et al., 1986 Nature 319, p. 791) or high velocity ballistic bombardment with metal particles coated with the nucleic acid constructs (Kline et al., 1987 Nature 327, p. 70; U.S. Pat. No. 4,945,050). Once transformed, the cells can be regenerated by those skilled in the art. Of particular relevance are the methods to transform foreign genes into commercially important crops, such as rapeseed (De Block et al., 1989 Plant Physiol. 91, pp. 694-701), sunflower (Everett et al., 1987 Bio/Technology 5, p. 1201), soybean (McCabe et al., 1988 Bio/Technology 6, p. 923; Hinchee et al., 1988 Bio/Technology 6, p. 915; Chee et al., 1989 Plant Physiol. 91, pp. 1212-1218; Christou et al., 1989 Proc. Natl. Acad. Sci. USA 86, pp. 7500-7504; EP 0 301 749), rice (Hiei et al., 1994 supra), and corn (Gordon Kamm et al., 1990 Plant Cell 2, pp. 603-618; Fromm et al., 1990 Biotechnology 8, pp. 833-839).

Those skilled in the art will appreciate that the choice of method can depend on the type of plant, i.e., monocotyledonous or dicotyledonous.

Methods of transforming monocotyledonous plants are for instance disclosed in U.S. Pat. No. 5,591,616. Methods of transforming dicotyledonous plants are for instance disclosed in U.S. Pat. No. 6,858,777.

In another embodiment, the vector as described herein can be directly transformed into the plastid genome. Plastid transformation technology is extensively described in U.S. Pat. Nos. 5,451,513, 5,545,817, and 5,545,818, in PCT application no. WO 95/16783, and in McBride et al., 1994 Proc. Natl. Acad. Sci. USA 91:7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation).

Agrobacterium tumefaciens cells containing a vector comprising an expression cassette of the present invention, wherein the vector comprises a Ti plasmid, are useful in methods of making transformed plants. Plant cells are infected with an Agrobacterium tumefaciens as described above to produce a transformed plant cell, and then a plant is regenerated from the transformed plant cell. Numerous Agrobacterium vector systems useful in carrying out the present invention are known. These typically carry at least one T-DNA border sequence and include vectors such as pBIN19 (Bevan, 1984 Nucleic Acids Res. 12: 8711-8721).

Methods using either a form of direct gene transfer or Agrobacterium-mediated transfer usually, but not necessarily, are undertaken with a selectable marker which can provide resistance to an antibiotic (e.g., kanamycin, hygromycin or methotrexate) or a herbicide (e.g., phosphinothricin). The choice of selectable marker for plant transformation is not, however, critical to the invention.

General methods of culturing plant tissues are provided for example by Maki et al., (1993, Methods in Plant Molecular Biology & Biotechnology, Glich, et al., (Eds. pp. 67-88 CRC Press, 1993); and by Phillips, et al., (1988, Corn & Corn Improvement, 3rd Edition; Sprague, et al., (Eds. pp. 345-387)).

After transformation the transgenic plant cells are placed in an appropriate selective medium for selection of transgenic cells which are then grown to callus. Shoots are grown from callus and plantlets generated from the shoot by growing in rooting medium. The particular marker used will allow for selection of transformed cells as compared to cells lacking the DNA which has been introduced.

To confirm the presence of the transgenes in transgenic cells and plants, a variety of assays can be performed. Such assays include, for example, “molecular biological” assays well known to those skilled in the art, such as Southern and Northern blotting, in situ hybridization and nucleic acid-based amplification methods such as PCR or RT-PCR; “biochemical” assays, such as detecting the presence of a protein product, e.g., by immunological means (ELISAs and Western blots) or by enzymatic function.

### Methods of Growing Plants

The methods of growing plants according to the present invention are based on a number of realizations that together form the basis of the hypothesis that the baseline respiration rate in plants is fixed early in the ontogeny of the plant. Without wishing to be bound by any theory, the following is contemplated herein:
i) respiration rates reached in a plant during early ontogeny culminate in the development of a baseline respiration rate, which is the minimum rate at which said plant will respire when mature;
ii) this baseline respiration rate is attained by one or more step-up increments and is ultimately fixed at baseline level by epigenetic changes;
iii) the step-up increments in the respiration rate are the plant's response to increased protein turnover rates which in turn are the result of oxidative stress experienced by said plant, and
iv) the step-up increments in the AOX component of the total dark respiration are the result of oxidative stress experienced by said plant.

Therefore, when attempting to keep the respiration rates in mature plants as low as possible, it is important to minimize the oxidative stress to said plant during the phase wherein the baseline respiration rate is fixed by epigenetic factors, that is, early in ontogeny. This can be achieved by manipulating the plants' growth environment, or by (genetically) manipulating the plant itself.

Hence, a method of growing plants according to the present invention comprises the important step of allowing a seedling, tissue culture or plantlet to develop into a plant and to pass through its early ontogenic phase wherein epigenetic factors fix the baseline respiration rate to a permanent minimum level. The early ontogenic phase wherein epigenetic factors fix the baseline respiration rate to a permanent level can differ for different plants, and can therefore not be defined for each and every plant species in advance. Also, the phase at which epigenetic factors determine the permanent minimum rate of respiration can vary between plant varieties within a species. This phase can however be experimentally determined for each and every plant species or plant variety as follows:

Seedlings, tissue cultures or plantlets are allowed to develop into mature plants. The total development time is recorded and divided into a large but practical number of regular intervals (between 2 and for instance 10, 20, 50, 100, or 1000 intervals). During each interval, the respiration rate of the developing plant is measured using standard techniques. After each measurement, the plant is temporarily subjected to oxidative stress, for instance by exposing it to ozone. The oxidative stress can optionally be applied at incrementally increasing levels during the different intervals. The early ontogenic phase wherein epigenetic factors fix the baseline respiration rate (in particular the AOX component) to a permanent level is now determined from the data obtained as:

a) the total number of intervals preceding the interval in which the respiration rate returns to a baseline level after said oxidative stress period, or

b) the total number of intervals during which the respiration rate shows a step-wise increase relative to the preceding interval and no decrease in the subsequent or following interval.

Roughly, in a method of the present invention the early ontogenic phase wherein epigenetic factors fix the baseline respiration rate to a permanent level is the prefloral stage, preferably a period from 0.1-6 months, preferably 0.5-6 months following the germination.

A method of growing plants according to the present invention further comprises the important step of preventing during early ontogeny of said plant the occurrence of an undesirable increase in the rate of respiration and/or protein turnover due to oxidative stress. It is to be understood that in accordance with the above-described model for epigenetic fixation of baseline respiration rates, an undesirable increase in the rate of respiration is equivalent to an undesirable increase in the rate of protein turnover. When use is made of the term “baseline respiration rate” herein, the term includes reference to the use of oxygen by said plant resulting from carbon dissimilation, the contrary of carbon fixation. The baseline respiration can be determined my methods described herein and includes the total dark respiration plus the oxidation via the alternative oxidation (AOX) pathway.

Oxidative stress is often, but not exclusively, the result of damage to the plant brought about by radiation or reactive oxygen species, including H₂O₂, O₃, and even SO₂, CO₂ and CO, in certain instances. Hence, the prevention of an undesirable increase in the rate of respiration and/or protein turnover due to oxidative stress can be attained by preventing exposure to radiation or reactive oxygen species that result in such an increase. Most notably, intracellular H₂O₂ levels and atmospheric O₃ concentrations are maintained at sub-stress levels. Such levels can differ between plant species and between plant varieties, but can be easily determined experimentally. For instance, a plant can be exposed to a certain test level of radiation or reactive oxygen species for a predetermined period of time (i.e. varying between several minutes to several weeks) and the rate of respiration and/or protein turnover is determine before and after said exposure. A sub-stress level of radiation or reactive oxygen species is the level at which the exposure does not result in an increase of the post-exposure rate relative to the rate before.

In embodiments of methods of the present invention, the oxidative stress is prevented by providing a transgenic plant that is capable of directly controlling the content of a reactive oxygen species in its growth environment such that they remain at sub-stress levels.

The growth environment can be the soil, substrate, medium or atmosphere in which the plant is grown. Preferably the content of the reactive oxygen species, most preferably O₃, is controlled in the growth atmosphere, most preferably in the air directly surrounding the leaves.

In order to prevent oxidative stress, the O₃-content of the growth atmosphere of the plant during the early ontogenic phase is preferably kept below 100 ppbv, most preferably below 75 ppbv, even more preferably below 60 ppbv, still more preferably below 50 ppbv, even more preferably below 40 ppbv, yet even more preferably below 35, 30, 25, 20, 15, 10, 5 or 1 ppbv, wherein ppbv refers to parts per billion by volume. Sporadic increases are likely not to have a major effect on the plants, yet values above 100 ppbv should essentially be avoided at all times during the early ontogenic phase.

As indicated, this can be attained by providing the plant with the (transgenic) capability of effective control of the level of reactive oxygen species via the (endogenous) production of a material that reacts with the reactive oxygen species in the atmosphere, thereby rendering the reactive oxygen species non-reactive. A suitable example of such a material is isoprene. Hence, the transgenic plant is preferably capable of producing terpene gases such as isoprene gas in order to remove reactive oxygen species from the air directly surrounding it. Generally, an amount of terpene capable of inactivating the equivalent of 1-1000, more preferably 50-150 parts per billion by volume (ppbv) of ozone is sufficient.

The production of terpenes such as isoprene in the transgenic plant of the invention, brought about by the terpene synthase gene, can be enhanced whenever necessary by the addition of deoxyxylulose (DOX) to the growth environment of the plant, either by direct spraying with DOX, by endogenous production of DOX in the plant, or by exogenous production of DOX for instance by using transformed microorganisms capable of producing and excreting DOX.

In principle, only the ROS content in the air that is in direct contact with the plant surface can be controlled. As indicated, this is most effectively attained by providing the plant with an endogenous source of terpene such as isoprene. To this end, isoprene can be endogenously produced in leaves of a plant. In plants that do not contain in their genome a (functional) biosynthetic pathway for the production of terpenes the missing genes for such a pathway can be introduced by providing a transgenic or recombinant plant transformed with said genes. One of said genes is the gene encoding the enzyme isoprene synthase. The skilled person is well aware of the various methods and techniques for producing such transgenic plants, and these are described in detail above. Reference can also be made to Sharkey et al. (2005 Plant Physiol. 137(2): 700-712) who describe the transformation of Arabidopsis (Arabidopsis thaliana) with the genomic isoprene synthase gene from kudzu (Pueraria montana) using the constitutive CAMV35S promoter. This disclosure is incorporated herein in its entirety by reference.

It is expressly noted that the CAMV 35S promoter is not suitable for use in aspects of the present invention. The Cauliflower Mosaic Virus (CaMV) is a double-stranded DNA virus which infects a wide range of crucifers, especially brassicas such as cabbages, cauliflowers, oilseed rape or mustard. The CaMV virus has two promoters (35S and 19S) from which transcription of its genes is driven by the plant's own molecular machinery. These promoters override the plant's own regulatory system, as they are constitutive, i.e. they are constantly switched on and can't be regulated or switched off by the plant. Hence, this feature renders the CaMV promoters, and particularly the 35S promoter unsuitable for the present purpose because the maintenance of a low AOX in transgenic plants is neutralized by an increased C-loss due to production of the anti-oxidant compound.

Transgenic plants and methods of growing plants according to the present invention have as an advantage that the net biosynthetic efficiency of said plant is increased during its entire lifetime. The invention is therefore especially important for plants that have a life cycle of multiple years, because it is in these plants that a high baseline respiration rate attained during early ontogeny has the most impact on the total production of the plant over its entire lifetime. Hence a plant in aspects of the present invention is preferably a perennial plant. However, also crop plants wherein in increase in the yield with a few percentages can be of great economical value are particularly considered herein. Thus, it will be appreciated that the methods of the present invention are particularly suitable for plants in which production parameters are very important, such as for instance in crop plants and plants projected to help counteracting atmospheric CO₂ accumulation for climate stabilizing purposes (carbon credits).

The present invention can be practiced on a large variety of plants. It is an advantage of the plants and methods of the present invention that the average yearly crop production is no longer adversely affected by seasonal fluctuations in the atmospheric load of reactive oxygen species, which normally have a lasting negative influence on plant growth.

It is another particular advantage that the planting or sewing season need not be selected such that in its early ontogenic phase as defined herein the plant's exposure to oxidative stress is minimized. It is now a feature of the plant itself that prevents the plant's AOX to be upregulated permanently during periodic exposure to undesirable or stress-level concentrations of atmospheric reactive oxygen species such as O₃, which, in particular in global areas where crop burning is practice seasonally, can have an enormous impact on plant yield.

Based on the teachings of the present invention, the skilled person will understand that certain planting seasons in Brazil and other parts of the world that suffer from high ozone levels during certain parts of the year, need no longer be avoided due to excessive atmospheric concentrations of reactive oxygen species, such as during sugarcane burning. In fact, the plants of the present invention can very suitably be used in such regions, and can even experience their early ontogenic phase during seasons that will be considered less suitable for non-transgenic crops.

Therefore in another aspect, the present invention provides a method for improving the production of a plant, comprising the steps of transforming a plant with a vector comprising a nucleic acid sequence encoding an anti-oxidant enzyme) or an enzyme that produces an anti-oxidant compound, wherein said nucleic acid sequence is operably linked to a regulated promoter functional during early ontogeny in said plant, so as to provide a transgenic plant.

Such a method can further comprise the steps of producing transgenic seed from said transgenic plant and sewing said transgenic seed so as to produce a transgenic seedling or plantlet and allowing the seedling or plantlet to develop into a transgenic plant, wherein during the early ontogenic phase of said transgenic plant an exposure to undesirable concentrations of reactive oxygen species is essentially prevented as a result of the expression of said vector in said transgenic plant.

The expression of said vector in said transgenic plant can be brought about by chemical induction. In such instances, the vector comprises as a regulatory element a promoter that is chemically regulated as described herein above. When the transgenic plant is treated with the inducing chemical, the anti-oxidant is produced. Hence, the method of the invention includes a method for growing plants comprising producing a transgenic plant comprising a vector that comprises a nucleic acid sequence encoding an enzyme that converts reactive oxygen species into a non-reactive or harmless (non-damaging) form, or that comprises a vector comprising a nucleic acid sequence encoding an enzyme that, when expressed in cells of said transgenic plant, result in the production of an anti-oxidant compound;

wherein said nucleic acid sequence is operably linked to a chemically inducible promoter; producing transgenic seed from said transgenic plant and sewing said transgenic seed so as to produce a transgenic seedling or plantlet and allowing the seedling or plantlet to develop into a transgenic plant, wherein during the early ontogenic phase of said transgenic plant said seed, seedling and/or plantlet is exposed to an effective concentration of the inducing chemical. Said effective concentration is defined herein as a concentration of the inducing chemical that activates the inducible promoter such that the nucleic acid encoding the enzyme is expressed in said transgenic plant, and wherein said expression is preferably at a level that results in an effective anti-oxidant level in said plant. Due to the chemical induction and resulting expression of the vector in said transgenic plant, the exposure to undesirable concentrations of reactive oxygen species is essentially prevented.

When reference is made herein to terms such as improving the production or increasing in the yield, it is meant that such attributes include or are brought about by an improved net carbon fixation efficiency, biomass production, dry matter content and/or pest resistance of said crop plant. All references cited in this specification are incorporated by reference herein in their entirety.

### EXAMPLES

### Example 1

As one example, the present invention can be performed as follows. In a greenhouse roses are grown under normal conditions of temperature, water, nutrients and light. In addition, the greenhouse atmosphere is controlled for the amount of ozone, for instance by using an ozone scrubber in order to reduce the amount of ozone in the greenhouse atmosphere. Furthermore, in order to avoid entry of ozone from the outside atmosphere, the greenhouse is well closed.

At least for the duration of the early ontogenic period as defined herein above, the ozone levels in the greenhouse are maintained at the lowest possible levels, preferably below 20-30 ppbv. Thereafter, that is after the early ontogenic phase, the plants can be grown under less stringent conditions or the plants can be brought outside the greenhouse for further growth.

The exposure to the beneficial growth environment will result in plants having a lower respiration rate when mature—compared to plants being exposed to high ozone environments, and such plants will exhibit higher production output as described herein above.

### Example 2

The Examples as described below demonstrate that the ontogenic phase of plants as described herein provides for a memory effect with respect to respiration via the AOX pathway. In particular it will be shown that the ontogenic phase is a learning period during which oxidative stress, such as provoked by ozone, irreversibly upregulates the non ATP rendering Alternative Oxidative Pathway or AOX component within the total dark respiration in ambients with higher ozone levels. Respiration via this AOX pathway is the “alternative oxidative pathway respiration”, which is also referred to as “alternative path respiration” (APR), “cyanide insensitive respiration” or “SHAM sensitive respiration”.

The tests, which were carried out in 3 ambients with different ozone levels, show that cotton plants at the end of their vegetative phase and upon entering the generative phase (i.e. in the ontogenic phase) have a defined and fixed maximum respiration efficiency expressed as [TDR-AOX]/[TDR], wherein TDR is the total dark respiration, and wherein AOX is the alternative oxidative pathway (AOX) respiration.

This respiration efficiency is set by the amount or level of ozone to which the plant is exposed in its ontogenic phase, herein referred to as the ontogenic phase level of ozone (OPLO). After the ontogenic phase, exposure of the plants to ozone levels higher than OPLO increases the AOX (AOX follows and upregulates). When the ozone level is subsequently returned to OPLO, the AOX also returns to the initial ontogenic level. Exposure of the plants to ozone levels lower than OPLO does not result in a lowering of the AOX (i.e. the AOX doesn't follow but maintains it's original level established during the ontogenic phase).

It was found that plants with higher respiration efficiency also have higher leaf weight (both when expressed as fresh and as dry weight) per surface area.

### Materials and Methods

### Plant Material

The following plants were used in the experiments: cotton (Gossypium spp.; dicotyledonous), bean (Phaseolus vulgaris L.; dicotyledonous), corn (Zea mays L.; monocotyledonous), sugarcane (Saccharum spp.; monocotyledonous), rice (Oryza sativa L.; monocotyledonous), soy (Glycine max L.; dicotyledonous) and wheat Triticum aestivum L; monocotyledonous). Cotton, bean, corn, rice, soy and wheat were provided in the form of seeds. Sugarcane (see also Example 3) was provided in the form of plantlets derived from tissue culture clones grown in agar.

### Planting

The seeds were planted in standard PE plastic flower vases no. 15 (height 15 cm, upper internal diameter of 15.5 cm, bottom internal diameter of 12 cm, with eight 10 mm drainage holes in the bottom). At the bottom of each vase a round piece of aquarium filter cloth of 12 cm diameter was placed in order to prevent gravel stones to wash out of the pot. 480 ml of washed stone gravel has been placed on top of the filter cloth. A second piece of filter cloth was placed on top of the gravel in order to separate it from the next layer above, and prevent the mixing of the two layers. The second layer consisted of 1.30 litres of a mixture of perlite and vermiculite (50/50% v/v), pre-saturated with 570 ml of nutrient solution (see below).

For all crops except sugar cane 8 selected seeds were planted per pot at the following depths: Cotton: 2 cm; beans: 2 cm; corn: 4 cm; rice: 1.5 cm; soy: 3 cm depth; wheat 2.5 cm. All pots were covered with a dome consisting of a transparent (PP) 1 litre vase turned upside down. The rim of this cover vase was neatly in contact with the inner wall of the planting pot. The seeds were allowed to germinate by placing the pots in the dark at 30° C. Cotton, beans and corn germinated first and were placed inside a phytotron three days after planting of the seeds and domes were removed the next day. Rice, soy and wheat germinated later and were placed inside a phytotron 5 days after planting with removal of the domes the next day.

For sugarcane 4 plantlets per pot were provided and these plantlets were transplanted from the agar following washing of their root system with nutrient solution (see below) in order to remove residual agar. Every sugar cane pot was provided with a dome consisting of a 5 litre water flask (PP) of which the bottom was removed creating a 30 cm high dome. Crepe-tape was used to provide an air-tight connection between the bottom rim of the flask and the planting pot. The 40 mm screw cap on top functioned as a ventilation valve, initially maintaining near 100% RV to prevent the plantlets from drying out. Directly after transplanting, the sugar cane plantlets were placed in a phytotron (4 pots per phytotron) more in particular in acrylic cabinets created therein (see below). The sugar cane domes were removed 10 days after transplanting.

All plantings were done at day 0. shifting plants to new ozone background for all plants=day 70.
Cotton: 0 ppb plants for 7 days at 60 ppb (final day at 60 ppb=day 77 when placed back in 0 ppb) recuperate in tree days (day 80). 60 ppb plants after 8 days (=day 78) in 0 ppb still act as if in 60 ppb.
Sugar Cane: plants from 0 ppb, after having their first 70 days of development in 0 ppb, stayed for the nex 33 days (till day 103) in 60 ppb before being placed back in their original 0 ppb. At day 103 their AOX level showed as high as of traditional 60 ppb grown plants but fully recuperated in 5 days. Plants grown at 60 ppb, at day 70 shifted to 0 ppb maintained their 60 ppb AOX levels even after 33 days (=at day 103) in 0 ppb.

### Nutrient Solution.

A single formulated nutrient solution was given to all crops during the full testing period containing 15 meq/l cations and 15 meq/l anions giving it an EC of 1.7 mS/cm. pH=5.8. Macro nutrients: K=4.50 mmol/l, Ca=3.75 mmol/l, Mg=1.50 mmol/l, N—NO=9.75 mmol/l, N—NH4=0 mmol/l, SO_{4=1.95} mmol/l, PO4=1.35 mmol/l. Micro nutrients: B=28 μmol/l, Zn-EDTA=13 μmol/l, Cu-EDTA=1.6 μmol/l, Mo=0.1 μmol/l, Fe-EDDHMA=52 μmol/l, Mn-EDTA=12 μmol/l. On all instances where water was required only distilled water was used. The nutrient solution was the only water source for the plants.

### Growth Conditions

Phytotron.

In order to create and maintain the desired climate in an ambient that physically supports the plants to be exposed and tested, three identical phytotrons were constructed. Each phytotron was designed in order to internally control: light level, air temperature, air humidity, air CO₂ level, air O₃ level and air circulation. Steel plated, electrostatically painted white modules with 50 mm and 100 mm thick isopore insulation designed for cold store construction were used to create three identical and hermetically sealed rooms of 4.0 m by 3.0 m by 2.5 m (l×w×h)=30 m³. Each room was equipped with a single sealed door for access.

Acryl Growing Cabinets.

Inside each phytotron two fully transparent growing cabinets (90 cm (deep)×120 cm (wide)×140 cm (height)) were provided (5-6 mm transparent acrylic plates). Each growing cabinet was further divided into four plant-compartments with acrylic plates, each plant compartment housing four pots with test plants. Each plant-compartment was vertically separated from a neighbouring compartment by a lamp-compartment housing 4 tube lights (Philips TLD 30W/75, 2000 lumen & 25 μmol/s) vertically positioned one above the other. The outside of the outermost plant-compartments was also fitted with a lamp-compartment for complete illumination of the compartment. A total of 40 Philips TLD 30 W/75 lamps were installed in each phytotron. Each plant-compartment was further fitted with four port holes (22×45 cm) that can be closed. The cabinets were positioned on a metal base giving them a ground clearance of 60 cm. The top of each cabinet was provided with a mirror facing inward to reflect upgoing light.

Light Control:

A Programmable Lighting Control (PLC) (model Logo 12/24 RC from Siemens) was used to control the photoperiod and lamp sequence. The acrylic cabinets were equipped with four levels of light counting 10 lamps each (40 lamps in total), numbered 1-4 from top to bottom. Level 1 was activated at 5:00 a.m. In sequence every next level came in one hour later providing full light at 8:00 a.m. At 8:00 p.m. all levels were deactivated at once providing full darkness.

Anti-Chamber.

A sealed central corridor gave access to the phytotrons and had the function of anti-chamber. The central corridor was kept at negative pressure in order to minimize air mixing between the phytotrons in the event of opening their doors. A blower fan at the end of the corridor was used to further minimize ozone translocation from high ozone to low ozone regime climates by moving the air from the lower ozone chambers to the higher ozone levels (e.g. from phytotron 1 (set point ═O ppbv O₃) towards phytotron 2 (set point=30 ppbv O₃) towards phytotron 3 (set point=60 ppbv O₃)).

CO₂ Control:

One central CO₂ interface & control system for CO₂ control was used (C-Control II, Conrad Electronic GmbH, Germany).

At 10 minutes interval the CO₂ concentration in each of the three phytotrons as well as in the outside ambient air was measured using a CO₂ analyzer (Priva 250E, (NDIR) gas analyzer, Priva, Holland).

Set point for CO₂ in each phytotron was set at 420 ppmvat 940 mBar and 26° C. during the photoperiod and 450 ppmvat 940 mBar and 26° C. for the dark period.

Before being interpreted, CO₂ measurements were pressure- and temperature-compensated by the central CO₂ interface & control system, corrected for atmospheric pressure variations, temperature variations in and outside the phytotrons and friction losses in the guiding tubes, electric selenoids and drying filters interconnecting the phytotrons with the central CO₂ interface & control system. The system allows for a 20 ppmvCO₂ hysteresis. In order to justify air renovation in case of excess internal CO₂ concentrations, principally during the dark period, outside air was also being evaluated and was taken to be at least 20 ppmvbelow the internal excess concentration for the system to activate the air exhaust system. In case of sub set point CO₂ concentrations, principally due to photosynthesis activity during the photoperiod, pure CO₂ was injected. The amount of CO₂ required was calculated and its addition was controlled by the central CO₂ interface & control system. Every phytotron was internally equipped with a security solid state CO₂ monitor (CDM4161A module, Figaro, Japan), which triggered an alarm at CO₂ levels over 1000 ppmv.

Internal Air Circulation.

Two internal circulation ventilators (model Master Fan Top, from Treviso Brazil) were used to ensure circulation of 15.5 m3 of air per minute in the growing cabinets.

Air Exhaust System.

An air exhaust system was used to lower the CO₂ concentration in phytotron by partially substituting the air in the phytotron with outside air. An axial blower fan (model Taurus-H from Ventisilva Brazil) was used to drive the exchange whereby an external particles pre filter (model SA-3085 from Inpeca Brazil) and six activated carbon gas-mask chemical end filters (model RC203 from Carbografite Brazil) were used in combination to guarantee the incoming air quality.

Temperature Control.

York, high wall split cooling system, model YJDA-12FS-ADA with 3.52 kW cooling capacity was used. Temperature was set to average 26° C. (+/−1.5° C.) after air-passage through the growing cabinets.

Humidity Control.

Inside each phytotron, a ventilated psychrometer ((Engineering dep. Crops Advance Brazil) was used to monitor its wet and dry bulb temperature in a micro-fan boosted forced air stream. At a temperature difference equal to or more than 3.0° C., the psychrometer activated two piezo-element water nebulizers (model Polaris Ion 35 W from Mallory of which the internal high tension “ion” generators have been removed) until the temperature difference restores to 2.0° C. or less. This translated to vapor pressure deficit values between 4 to 8 mBar and relative humidity values between 75% to 85% within a temperature interval of 22° C. being close to the evaporater unit outlet, to 27° C. at the outlet of the acryl growing cabinets. Condensation water draining from the evaporator unit inside each phytotron was captured for recirculation. It was passed through a UV-C sterilization unit (Atman UV-5W equipped with a Philips TUV PL-S 5W UV-C lamp) before being collected in a 40 litres collection reservoir. The collection reservoir was equipped with a submerged pump (Atman HF-750 18 W) to supply the interconnected water reservoirs of the nebulizers with water in order to guarantee minimum water levels. At minimum level, an electronic level control system activates the pump. At full capacity, the collection reservoir drains into an external jerry-can.

Ozone Control.

Ozone generators (model OP-35-3P, Grupo Interozone, Brazil) were coupled to ozone monitors (model 202, 2B Technologies, Inc. USA; dynamic range of 1.5 ppbv to 100 ppmv of ozone; precision 1.5 ppbv or 2% of the reading) using a ozone monitor—generator interface (Engineering dep. Crops Advance Brazil). At full light level (all 40 lamps activated) and no reactive surfaces other than the internal physical phytotron structure present to interfere, the background ozone level inside the phytotrons did not exceed 3 ppbv ozone. This was used as the upper limit for phytotron number one. Phytotron number two was set at 30 ppbv average ozone. Phytotron number three was set at 60 ppbv average ozone. Ozone levels of phytotron number two and three were permanently measured at 10 second intervals and averaged per minute. Each ozone monitor fed averaged values over an analog output to an ozone monitor—ozone generator interface. Hysteresis was set at +/−5 ppbv. Ozone levels were monitored close to the inlet of the two internal circulation ventilators. The ozone generators were adapted to accept an external digital signal from the interface.

Closed Circuit Video Camera System.

Every phytotron was equipped with a video camera (Panasonic SDR-S7) sending its images over a TecVoz TEC30/04LIG2 digital video record system to a server CPU, recording all images. Over the test period the cameras have been manually put into position to record the plants in test.

Respiration//Gas Exchange Measurements

Respiration of plant material was tested as follows: Partial oxygen pressure drops due to oxygen consumption as result of dark respiration were measured using phase shift fluorescence measurement. For this, a NeoFox™ Phase Measurement System was used (Fluorometer Bench version using a QBIF600-VIS-NIR laboratory-grade bifurcated optical fiber with FOXY-R-8 cm overcoated fluorescence oxygen needle sensor, Ocean Optics Inc. USA).

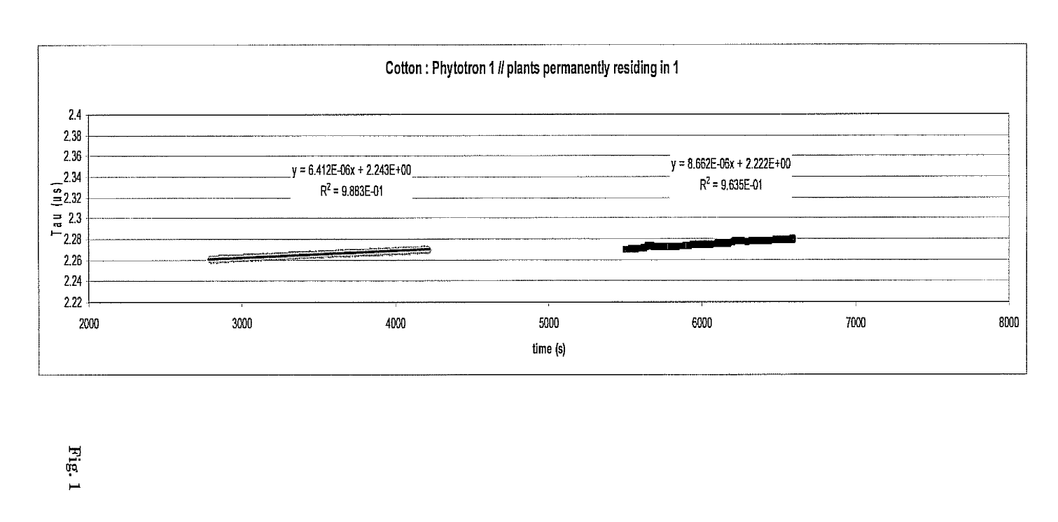

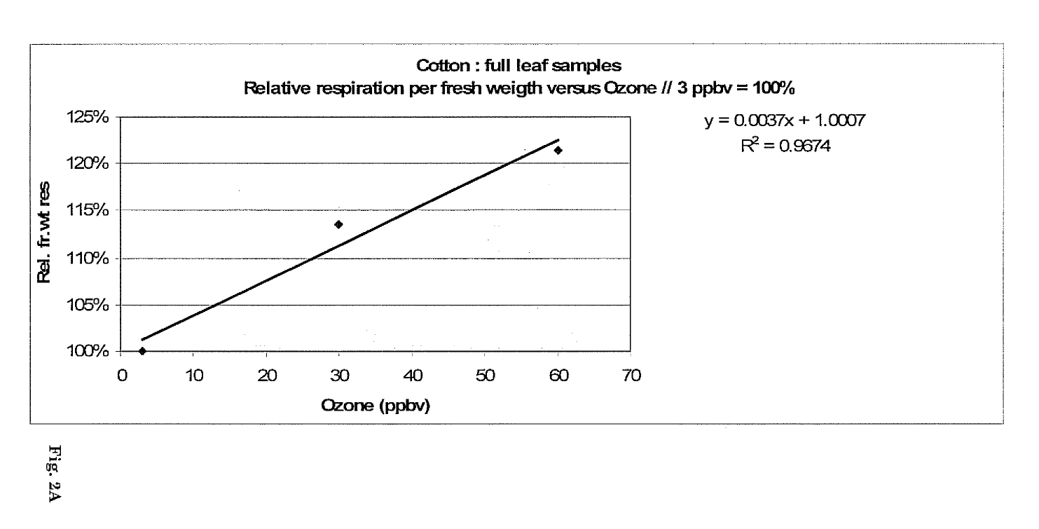

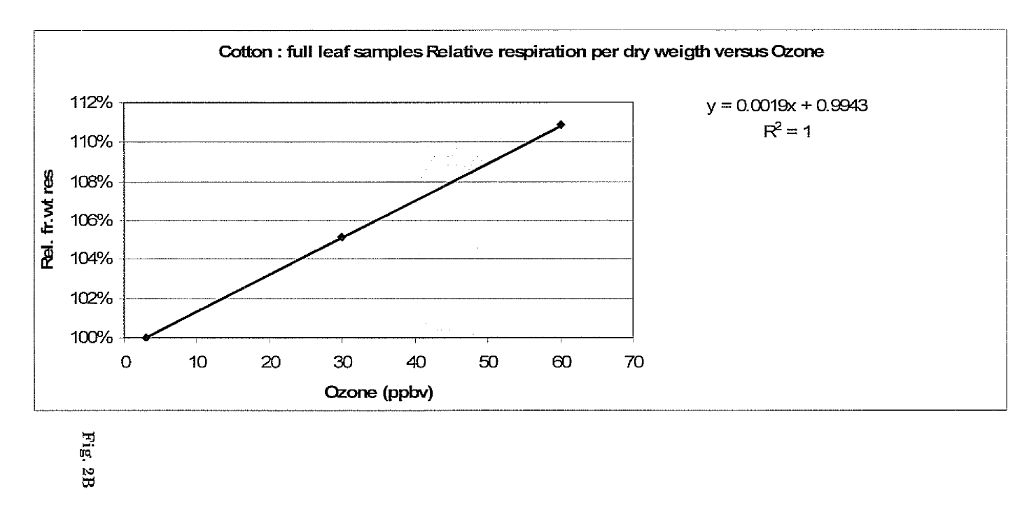

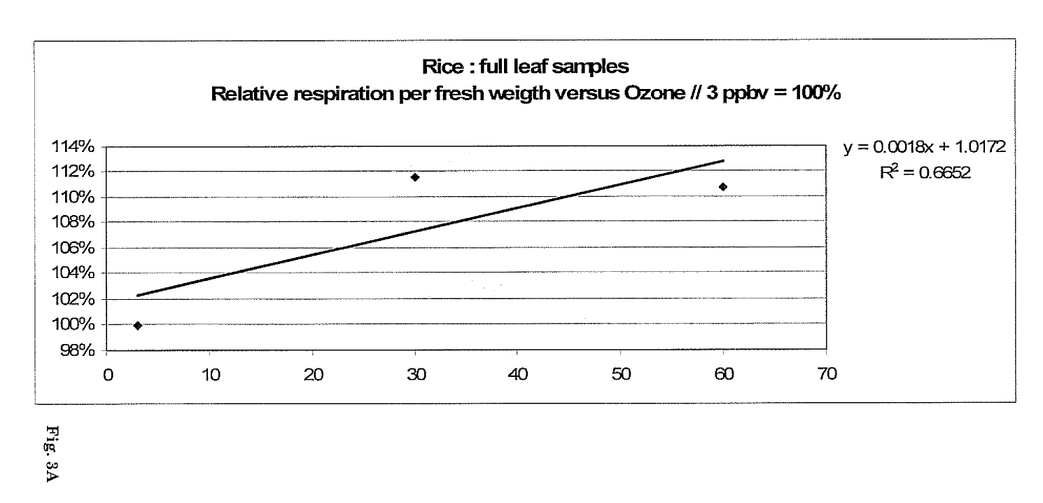

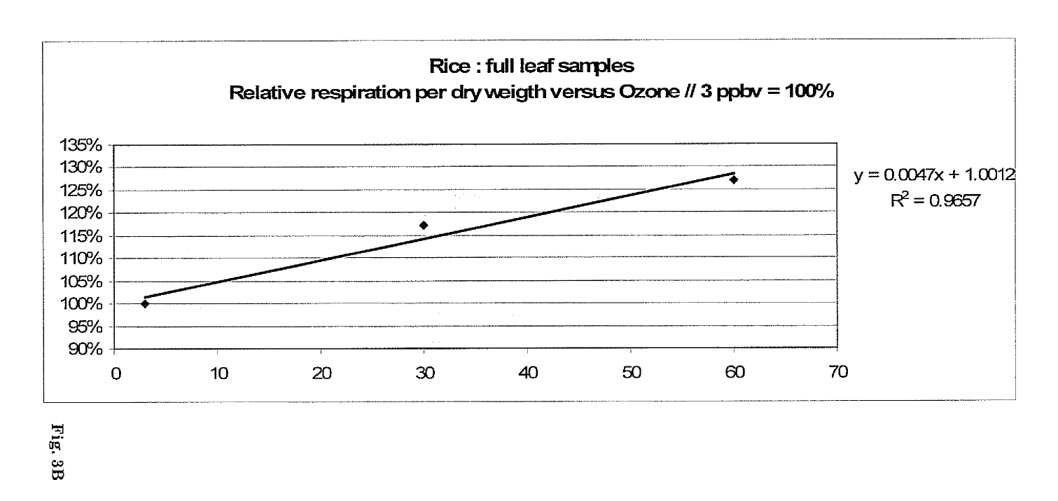

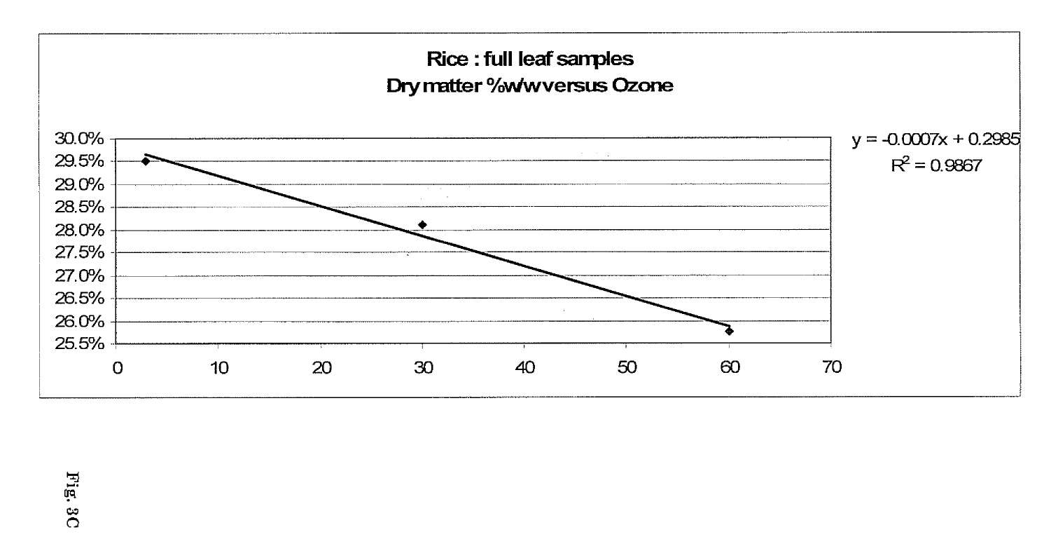

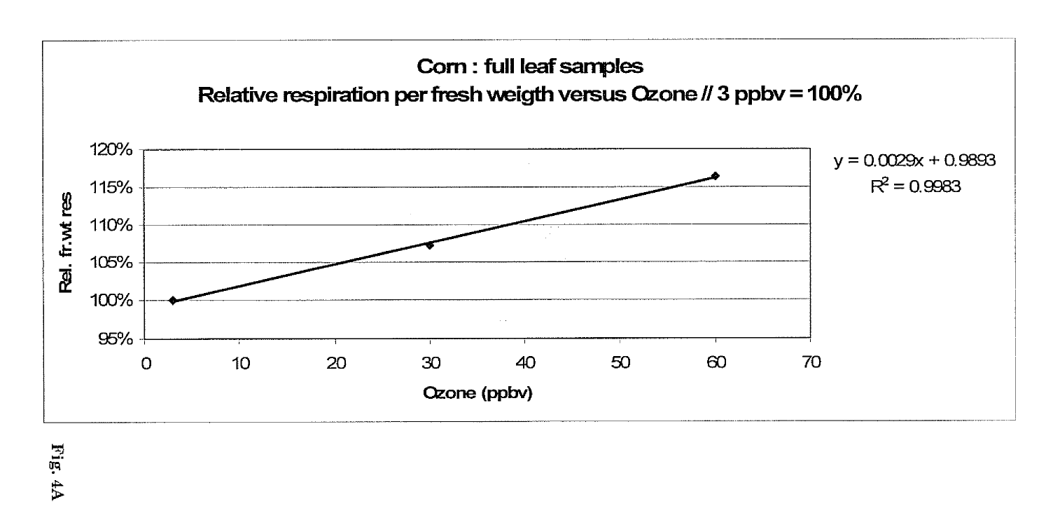

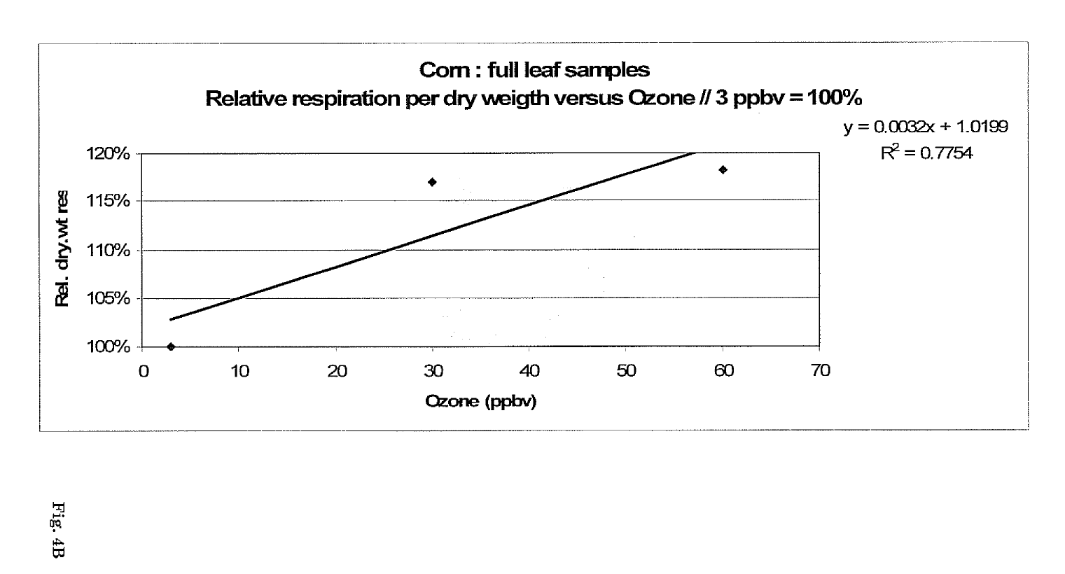

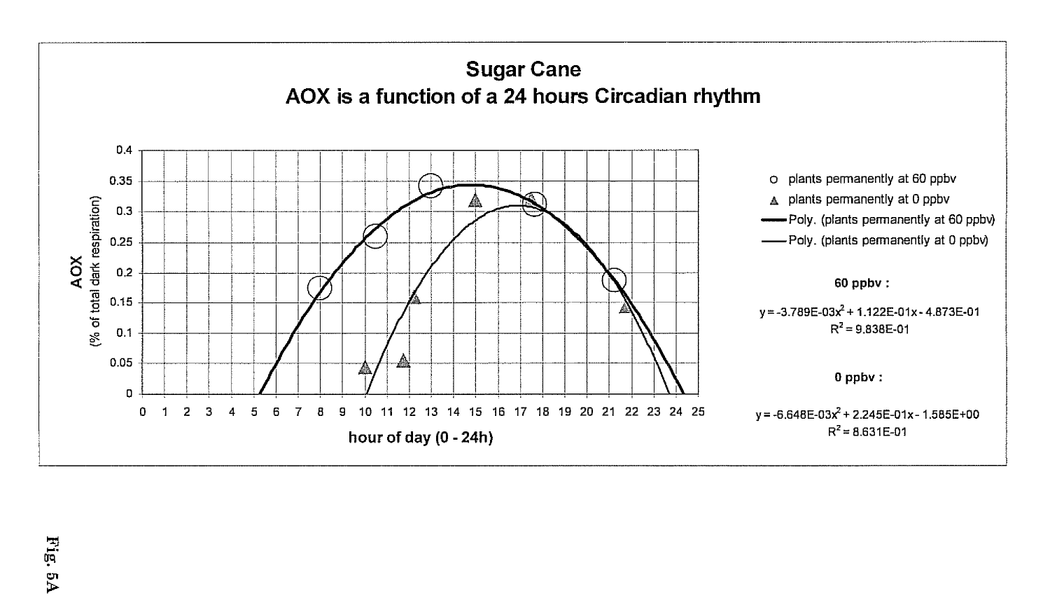

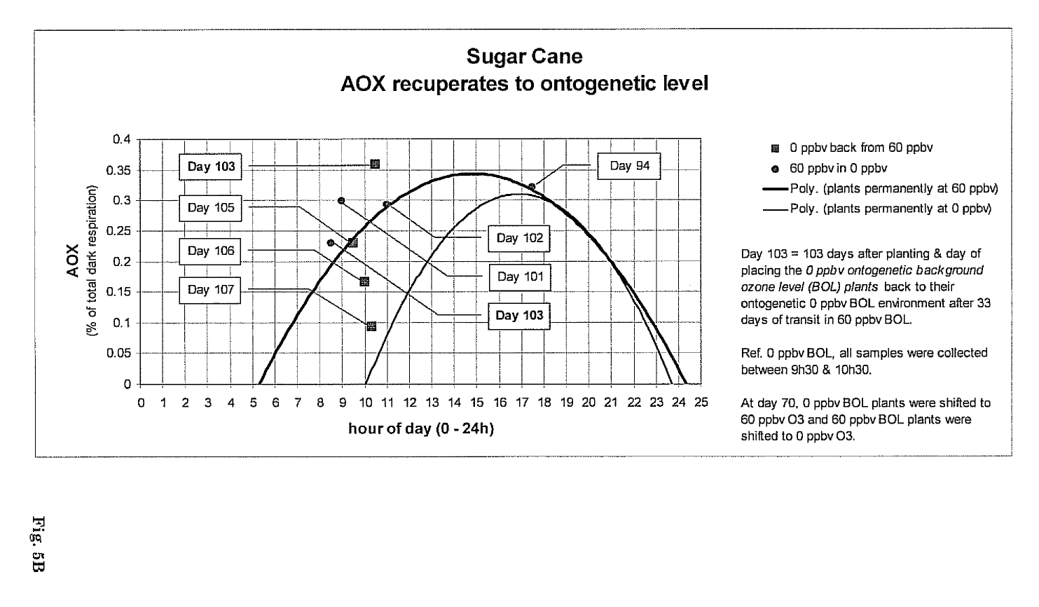

Fluorescence phase shift was expressed as Tau in microseconds (μs) and has an inverse and linear relation with the partial oxygen pressure. The sensor had an atmospheric volume fraction gain of O₂ per −1 μs Tau=23.79% at 26.0° C. and 936 mBar. An exemplary graph of cotton is presented in FIG. 1.

Salicylhydroxamic acid (SHAM) (99%, Sigma-Aldrich Brasil Ltda. Sao Paulo, Brazil) was used as an AOX inhibitor. The soluble sodium salicylhydroxamate salt was prepared by titrating SHAM with 0.1 M sodium hydroxide until neural pH. A stock solution (0.136 M) was prepared containing 300 ml/L ethanol from initial dissolving SHAM (same abbreviation is used for the salicylhydroxamate salt). From the stock solution a 20 mmol/L salicylhydroxamate final test solution was prepared by taking 12.3 ml stock solution and completing till 100 ml with a phosphate buffer solution (pH 7). The final SHAM test solution thus contained 3.7% residual ethanol. For proper control, the comparative test solution was also provided with 3.7 ml of ethanol per 100 ml of phosphate buffer solution (pH 7). To both solutions two drops of wetting agent (Silwet L-77 AG, GE Silicones Inc. USA) were added per 25 ml of test solution.

Plant material collected was always the first fully grown leaf counting from the apical point downwards or outwards (e.g. for monocotyls). For cotton this resulted in the third leaf under the apical point. Per test, four leafs from four different plants were sampled. Cotton leafs were cut in half along the mid nerve. Using a clipper, 16 leaf sections of equal surface area of 400.9 mm2 each were cut, one from each base half and one from each top half part giving 4 sections per leaf and 16 sections per test. The leaf sections were distributed equally over two sub samples, one with and one without salicylhydroxamate. Using a chirurgical blade, all sections were given 7 parallel cuts (through and through) at approximately 2.5 mm apart over almost the full length of the section to guarantee lateral infiltration of the test solutions and gas exchange.

Within 10 minutes of plant sampling the sections were cut, initial subsample weight was registered using a high precision laboratory scale and the subsamples were placed in their solutions with and without salicylhydroxamate for 60 minutes in the dark, to guarantee dark adaptation.

Each sample contained 4 leaves, 4 sections per leaf providing 16 sections, 8 per subsample. In 1 subsample of 8 sections AOX was inhibited, the other subsample served as control. Thereafter each subsample material was tissue dried and stacked using filter paper sections cut with the same clipper to separate individual leaf sections. Each subsample consisted of 9 filter sections and 8 leaf sections. Each stacked subsample was placed inside a closed container with a volume of 10 ml having only a single opening for entry of the oxygen sensor probe (a sterile 50 ml normal tip syringe (Embramac, Campinas Brazil) was used and the internal volume was set at 10 ml bruto by moving the plunger).

As stated, the drop in partial oxygen vapour pressure due to oxygen consumption as result of dark respiration was measured using phase shift fluorescence measurement.

Both containers containing the plant material to be measured (SHAM and control) were placed in a 16 litres temperature-stabilized insulated water bath with forced water circulation equipped with a temperature control unit (C-Control II, from Conrad Electronic GmbH, Germany) using a high precision temperature sensor interface set at 26.00° C. and maintaining the water temperature within +/−0.03° C. by calculating and applying the necessary heat injection through a 25W glass coated resistor element (Engineering dep. Crops Advance Brazil).

The oxygen needle probe from the NeoFox™ Phase Measurement System was inserted into the tip of the container (the 50 mL syringe), the opening was sealed and the complete setup was submerged in the water bath. The containers were given 60 minutes of temperature stabilization before valid readings were taken. The SHAM sample was read first and readings were taken every second. The readings in general tended to linearly stabilize within 15 to 20 minutes. Up to 20 minutes of stable trajectory was registered before switching the containers and the stabilizing and reading processes were repeated for the non-SHAM subsample.

Between measurements of the SHAM and non-SHAM subsample, the probe was allowed to stabilize. The readings were collected into computer memory.

From this, the Total Dark Respiration (TDR) including the AOX (non-SHAM) and Total Dark Respiration (TDR) excluding the AOX (SHAM) were determined. The results for cotton are provided in Table 1.

TABLE 1
Total dark respiration and salicyl hydroxamate (SHAM)-sensitive
AOX as measured by Phase shift fluorescence measurement - Cotton

| | | | | | AOX/TDR |
|---|---|---|---|---|---|
| | TDR excl AOX | TDR incl AOX | AOX | | relative to |
| | dTau/t (μs/s)⁸ | dTau/t (μs/s)⁸ | component | | initial |
| Situation | without SHAM | with SHAM | (arb units) | AOX/TDR | equilibrium |
| 0 ppbv ozone¹ | 6.41E−06 | 8.66E−06 | 2.25 | 26.0% | 100.0% |
| 0 →30 ppbv ozone² | 7.51E−06 | 1.08E−05 | 3.26 | 30.3% | 116.5% |
| 0 →60 ppbv ozone³ | 5.75E−06 | 9.52E−06 | 3.77 | 39.6% | 152.5% |
| 0 →60 →0 ppbv ozone⁴ | 8.87E−06 | 1.18E−05 | 2.95 | 25.0% | 96.2% |
| 30 ppbv ozone⁵ | 6.96E−06 | 9.93E−06 | 2.97 | 29.9% | |
| 60 ppbv ozone⁶ | 6.03E−06 | 9.36E−06 | 3.33 | 35.6% | 100.0% |
| 60 →0 ppbv ozone⁷ | 7.13E−06 | 1.11E−05 | 3.97 | 35.8% | 100.5% |

¹plants grown in 0 ppvb O₃;
²plants grown in 0 ppvb O₃ and transferred to 30 ppvb O₃;
³plants grown in 0 ppvb O₃ and transferred to 60 ppvb O₃;
⁴plants grown in 0 ppvb O₃, transferred to 60 ppvb O₃ and transferred back to 0 ppvb O₃;
⁵plants grown in 30 ppvb O₃;
⁶plants grown in 60 ppvb O₃; plants grown in 60 ppvb O₃ and transferred to 0 ppvb O₃.
⁸Values are the slope of the measurement curve as indicated in FIG. 1

In addition, full leaf Total Dark Respiration (TDR) measurements without the use of any pre-treatment solution (no SHAM and pretreatment control) were executed for cotton, corn and rice. Data (tables 2, 3 and 4) show clear positive correlation between a) total respiration per unit of fresh weight and ozone level, and b) total respiration per unit of dry weight and ozone level.

TABLE 2
Full leaf Cotton Total Dark Respiration (TDR) measurements

| | Ozone concentration during growth (ppbv) | | |
|---|---|---|---|
| | 0 | 30 | 60 |
| Normalized dark | 82.4 | 72.6 | 67.9 |
| respiration activity (fwt)¹ | | | |
| Normalized dark | 14.1 | 13.4 | 12.7 |
| respiration activity (dwt)² | | | |

¹As h/Tau/g fresh weigth/40 ml,
²As h/Tau/g dry weigth/40 ml

TABLE 3
Full leaf Rice Total Dark Respiration (TDR) measurements

| | Ozone concentration during growth (ppbv) | | |
|---|---|---|---|
| | 0 | 30 | 60 |
| Normalized dark | 32.6 | 29.2 | 29.4 |
| respiration activity (fwt)¹ | | | |
| Normalized dark | 9.6 | 8.2 | 7.6 |
| respiration activity (dwt)² | | | |

¹As h/Tau/g fresh weigth/40 ml,
²As h/Tau/g dry weigth/40 ml

TABLE 4
Full leaf Corn Total Dark Respiration (TDR) measurements

| | Ozone concentration during growth (ppbv) | | |
|---|---|---|---|
| | 0 | 30 | 60 |
| Normalized dark | 83.9 | 78.3 | 72.1 |
| respiration activity (fwt)¹ | | | |
| Normalized dark | 17.2 | 14.7 | 14.5 |
| respiration activity (dwt)² | | | |

¹As h/Tau/g fresh weigth/40 ml,
²As h/Tau/g dry weigth/40 ml

### Conclusions

The plants that passed their ontogenic phase in the 0 ppbv ozone ambient have an AOX respiration rate of 26.0% of the total 100% Total Dark Respiration rate. When placed in the 30 ppbv ozone ambient, the AOX respiration increased to 30.3%, +16.5% compared to the initial AOX respiration rate and those placed in the 60 ppbv ozone ambient increased their AOX respiration to 39.6%, +52.5% compared to the initial AOX respiration rate.

When returned to the 0 ppbv ozone ambient in which the plants lived their ontogenic phase, the AOX respiration of the plants which had passed time in the 60 ppbv ozone ambient returned fully to its initial level.

Plants grown up in the 60 ppbv ozone level ambients did not lower their AOX respiration during their stay in the zero ozone level ambient and maintained their high AOX respiration rate.

The AOX respiration rate expressed as percentage of the total dark respiration is defined as the base level during the ontogenic phase.

Plants with AOX levels defined during the ontogenic phase increase their AOX respiration when exposed to oxidative stress induced by higher ozone levels, but when the oxidative stress is reduced, cotton showed its AOX respiration rate to fully return to base level within 3 days. When exposed to ozone levels lower than the base level of the plants, the AOX respiration does not go below the base level defined during the ontogenic phase.

These findings have great implications for methods for preventing inefficient respiration in plants, and hence for increasing plant yield.

### Example 3

Sugarcane plants were grown in the same manner as described above in Example 2. The AOX and total dark respiration were measured for plants exposed to various levels of ozone as described above in Example 2. The results of the experiments are provided in FIGS. 5A-5D.

As shown in FIG. 5A, the AOX level is not static over the day, but rises and falls in response to the light regime (first light 5:00 am, last light 08:00 pm) according to a 24 hrs circadian rhythm. The plants grown in their early ontogenic phase under 60 ppbv of ozone exhibit a curve wherein the AOX rises earlier. Although the AOX need not necessarily be higher at any one time point, it is the total area below the curves that is the largest for the 60 ppbv grown plants.

As shown in FIG. 5B, plants grown in their early ontogenic phase in 60 ppbv of ozone, and returned to 0 ppbv of ozone maintain their high AOX, and follow the curve of the 60 ppbv of ozone plants (i.e already exhibit high AOX values at between 08:00 am and 11:00 am). However, plants grown in their early ontogenic phase in 0 ppbv of ozone, temporarily (33 days) placed at 60 ppbv after 70 days of 0 ppbv, and then returned to 0 ppbv of ozone, first exhibit a high level of AOX at about 10:00 am (approximately 35% of TDR), but eventually (after 5 days) return to their normal curve of a low AOX at 10:00 am (approximately 5% of TDR).

As shown in FIG. 5C, this recuperation after temporary exposure to 60 ppbv following the completion of the ontogenic phase under 0 ppbv, takes about 5 days.

As shown in FIG. 5D, AOX absolute values expressed as [dTau/dt] in picos/s as a function of a 24 hours circadian rhythm indicate the that upon recuperation the plant returns to a respiration rate wherein the AOX component is again greatly reduced.

The conclusions of this experiment are that sugarcane AOX, relative to Total Dark Respiration and in absolute values follows a 24 hour circadian rhythm.

Plants with 70 days of ontogenetic 0 ppbv background ozone level (BOL) and successively 33 days of transit in 60 ppbv BOL, increase their AOX to levels equal to plants residing permanently in 60 ppbv BOL and recuperate their ontogenetic low level of AOX within 5 days after being shifted back to 0 bbpv BOL.

Plants with 70 days of ontogenetic 60 ppbv BOL and thereafter 33 days of transit in 0 ppbv BOL exhibit consistently high AOX levels, coinciding with reduced yields.

The tables below indicates that the effect can be substantial.

TABLE 5
Comparison of relative AOX

| | | integration of |
|---|---|---|
| | Time of | the parabolic |
| | Day | function yields: |
| 60 ppbv | 5.3 | 0.0% |
| | 24.3 | 436% |
| 0 ppbv | 10.1 | 0.0% |
| | 23.7 | 283% |

| | |
|---|---|
| AOX 60 ppbv/AOX 0 ppbv (%/%) = | 154% |

TABLE 6
Comparison of absolute AOX

| | | integration of |
|---|---|---|
| | Time of | the parabolic |
| | Day | function yields |
| 60 ppbv | 5.6 | 0.0 |
| | 23.0 | 51.9 |
| 0 ppbv | 10.4 | 0.0 |
| | 23.3 | 30.7 |

| | |
|---|---|
| AOX 60 ppbv/AOX 0 ppbv (dTau/dt/dTau/dt) = | 169% |

TABLE 7
Production gain due to Assumption for CUE for 0 ppbv
background ozone level environment versus consequently
productivity relative to 60 ppbv environment

| | Gross C | | | Relative | |
|---|---|---|---|---|---|
| CUE | intake | Net C | Net C | Production | Production |
| at 0 | (0 ppbv = | uptake | uptake | 0 over | GAIN 0 |
| ppbv | 60 ppbv) | 0 ppbv | 60 ppbv | 60 ppbv | over 60 ppbv |
| 65% | 568 | 369 | 321 | 115% | 15% |
| 60% | 497 | 298 | 250 | 119% | 19% |
| 55% | 442 | 243 | 195 | 125% | 25% |
| 50% | 397 | 199 | 150 | 132% | 32% |
| 45% | 361 | 163 | 114 | 142% | 42% |
| 40% | 331 | 132 | 84 | 158% | 58% |
| 35% | 306 | 107 | 59 | 183% | 83% |
| 30% | 284 | 85 | 37 | 232% | 132% |
| 25% | 265 | 66 | 18 | 371% | 271% |
| 20% | 248 | 50 | 1 | 3828% | 3728% |

CUE = Carbon Use Efficiency = net 24 h C utilisation of gross C intake of photosynthesis (Obs: CUE according to literature varies from 20% to 65%)

SEQUENCE LISTING

SEQ ID NO: 1 (amino acid sequence of the enzyme isoprene synthase
in Populus alba)

| | |
|---|---|
| 1 | matellclhr pislthklfr nplpkviqat pltlklrcsv stenvsftet etearrsany |
| 61 | epnswdydyl lssdtdesie vykdkakkle aevrreinne kaefltllel idnvqrlglg |
| 121 | yrfesdirga ldrfvssggf davtktslhg talsfrllrq hgfevsqeaf sgfkdqngnf |
| 181 | lenlkedika ilslyeasfl alegenilde akvfaishlk elseekigke laeqvnhale |
| 241 | lplhrrtqrl eavwsieayr kkedanqvll elaildynmi qsvyqrdlre tsrwwrrvgl |
| 301 | atklhfardr liesfywavg vafepqysdc rnsvakmfsf vtiiddiydv ygtldelelf |
| 361 | tdaverwdvn aindlpdymk lcflalynti neiaydnlkd kgenilpylt kawadlcnaf |
| 421 | lqeakwlynk stptfddyfg nawksssgpl qlvfayfavv qnikkeeien lqkyhdtisr |
| 481 | pshifrlcnd lasasaeiar getansvscy mrtkgiseel atesvmnlid etwkkmnkek |
| 541 | lggslfakpf vetainlarq shctyhngda htspdeltrk rvlsvitepi lpfer |

SEQ ID NO: 2 (nucleotide sequence of the enzyme isoprene synthase in
Populus alba)

| | |
|---|---|
| 1 | gctctagagc atggcaactg aattattgtg cttgcaccgt ccaatctcac tgacacacaa |
| 61 | actgttcaga aatcccttac ctaaagtcat ccaggccact cccttaactt tgaaactcag |
| 121 | atgttctgta agcacagaaa acgtcagctt cacagaaaca gaaacagaag ccagacggtc |
| 181 | tgccaattat gaaccaaata gctgggatta tgattttttg ctgtcttcag acactgacga |
| 241 | atcggtacgt acgtaaaatc tgcaggaaaa ctcccccttc tctttttctt gtattttttt |
| 301 | tcatttacaa gttctttgga ttagcctgat cctccatttc tttcatttag tgtaatagga |
| 361 | tttgaaatat ggagttataa ccatggtttt cagaccccgc ctggactctt actcgggaaa |
| 421 | aaactcagat tattaggtta ccgagttaac cacgagttat caggtcaatc atggttaatc |
| 481 | attctcatca aaacaatgtt attctattct tttttcttga aaaaacaact catggactga |
| 541 | gtttgaatca gatcaccagc tctaggccgt gggttcactg gatcggccat cggacctagt |
| 601 | cgagtttatt aacaagatta cttttatttt attttggctt agtagtttgc ttagctaatt |
| 661 | ataaattctg tctagaatac tggtctttca atacatatat cttgtgactg tttgcatttt |
| 721 | tttcttctaa gtttcatcct tgttaaaact taaaggtgac cttactaaga taaaccaaca |
| 781 | cactccttta atttttaaat atagtaagat gatactataa aaaaatactt tctacttagc |
| 841 | gatctcattt gaatttgtca atccaagatt cctggatttc ttaatatatc cccagatctt |
| 901 | caaagtggat ccgactcttc gttagcttgt ggctaagtga gtcacactta cagctactgc |
| 961 | tccatatgga ccgtgcgtgg cctgcatatg atggcgctgg ttccatcgcc catttttgtg |
| 1021 | aaaatcagcc tagctggtca attcagatta acctgccata tgtcaaaatt aaaatgaaga |
| 1081 | gggttgtgaa gaagagaaaa ggaagaagct ctgtagtcat caatcaatct ccaatttcta |
| 1141 | actttgcata tactgtgatc ttgtagattg aagtatacaa agacaaggcc aaaaagctgg |
| 1201 | aggctgaggt gagaagagag attaacaatg aaaaggcaga gtttttgact ctgcttgaac |
| 1261 | tgatagataa tgtccaaagg ttaggattgg gttaccggtt cgagagtgac ataaggagag |
| 1321 | ccctcgacag atttgtttct tcaggaggat ttgatggtgt tacaaaaact agccttcatg |
| 1381 | ctactgctct tagcttcagg cttctcagac agcatggctt tgaggtctct caaggtacgt |
| 1441 | atttagttac ttacctcaat aatttaatat atccttatct atgaagtata ttcatgtagc |
| 1501 | atgcccttgc catcaacaga agcgttcagt ggattcaagg atcaaaatgg caatttctcg |
| 1561 | gaaaacctta aggaggacac caaggcaata ctaagcctat atgaagcttc atttcttgca |
| 1621 | ttagaaggag aaaatatctt ggatgaggcc agggtgtttg caatatcaca tctaaaagag |
| 1681 | ctcagcgaag aaaagattgg aaaagagctg gccgaacagg tgaatcatgc attggagctt |
| 1741 | ccattgcatc gcaggacgca aagactagaa gctgcttgga gtattgaagc ataccgtaaa |
| 1801 | aaggaagatg caaatcaagt actgctagaa cttgctatat tggactacaa cacgattcaa |
| 1861 | tcagtatacc aaagagatct tcgcgagaca tcaaggtcag tccaagaaca aaatttcacg |
| 1921 | ttcttaccaa ggtctagaaa gcaggtcaat tagatttctg tcagtggaaa ataactttgc |
| 1981 | tcctgctgtg tcactcaggt ggtggaggcg agtgggtctt gcaacaaagt tgcattttgc |
| 2041 | tagagacagg ttaattgaaa gcttttactg ggcagttgga gttgcgttcg aacctcaata |
| 2101 | cagtgattgc cgtaattcag tagcaaaaat gttttcattt gtaacaatca ttgatgatat |
| 2161 | ctatgatgtt tatggtactc tggatgagct ggagctattt acagatgctg ttgagaggtt |
| 2221 | tgtaccaaga aagaattaac ccctcaccat ttcggttcat catgttgtta agatgctaat |
| 2281 | cattttattt ccctcactat tcctggttgt ttcagatggg atgttaacgc catcaatgat |
| 2341 | cttccggatt atatgaagct ctgcttccta gctctctaca acactatcaa tgagatagct |
| 2401 | tatgacaatc tgaaggacaa gggggaaaac attcttccat acctaacaaa agcggtacag |
| 2461 | tataaatttc atcattattt gcagaaaatt aacatagcag gcttgcaatt ttcgattgct |
| 2521 | aaccgatctc tgttgtaatg tcttcgttcc tacagtgggc agatttatgc aatgcattcc |
| 2581 | tacaagaagc aaaatggctg tacaataagt ccacaccaac atttgatgac tatttcggaa |
| 2641 | atgcatggaa atcatcctca gggcctcttc aactaatttt tgcctacttt gccgtggttc |
| 2701 | aaaacatcaa gaaagaggaa attgaaaact tacaaaagta tcatgatatc atcagtaggc |
| 2761 | cttcccacat ctttcgtctt tgcaacgacc tggcttcagc atcggtaagt tcttcatatg |
| 2821 | cctgaatgta caggcctgta gctatccgtt cccgcatttt aacatttccg tttgcttatc |
| 2881 | caggctgaga tagcgagagg tgaaactgcg aattccgtat cctgctacat gcgtacaaaa |
| 2941 | ggcatttctg aggaacttgc tactgaatct gtaatgaatt tgatcgacga aacctggaaa |
| 3001 | aagatgaaca aagaaaagct tggtggctct ttgtttgcaa aaccttttgt cgaaacagct |
| 3061 | attaaccttg cacggcaatc ccattgcact tatcataacg gtgatgcgca tacttcacca |
| 3121 | gacgagctaa ctaggaaacg tgtcctgtca gtaatcacag agcctattct accctttgag |
| 3181 | agataaaagt aacaggtttt ccatgttgtc gtctgcaaga acaaataaca tatgctgcgt |
| 3241 | agaaaattaa gccatgtaaa taggctttaa ctccatgtcc ggcggagttt ttgcagcagc |
| 3301 | aagtaccctc cgcggccgca |

SEQ ID NO: 3 (Pyk10 promoter from Arabidopsis thaliana)

| | |
|---|---|
| 1 | gatctttcag agaaaaaaaa taaatttttt ttgacaaacg tagtgctaaa ctaaaccgta |
| 61 | aaaaaaaagg aaaaaaatgt cccttattca gatttccttt tgtaacccac acacatagct |
| 121 | aaactaattt acatcataat taaccactaa ccagtgtcac gaccttgctt cattggtctt |
| 181 | aaaaaggtcc atgtagggtc gtcagaaaag taaaaaagaa ttataatgca ataggattaa |
| 241 | ttatccaatt agctgattaa gtctaaatca agctgtctaa gtggtgacga aaacaaaaca |
| 301 | agcttattca acactagatt tgttaattgg attattgaaa ttgtaatgaa atgacgagtg |
| 361 | gttgatgaat aaagggaaat taatgttatt taataaataa ataaataaaa tcatcacagg |
| 421 | cgtatcggat ctgtgactaa aatcaattat tggctctgtt atactgttac taatcaatat |
| 481 | caaagaatag atttatgcct tcttgccatt ctcagtgcca ctgaaaaagt tttttcctat |
| 541 | ctaatttatt tttgttccaa atattaattc aaaccataaa atatgtatat gctacatatg |
| 601 | cagtgagaca tttaatgatc gaaggagcca ttgattgaac acaattagga acaccatgca |
| 661 | tcttatctac aatttccaat atcttcttgt aatactcaaa gtcaaaagat tggatctaca |
| 721 | atctgacgaa agaaataaag aaacgcttac acagtctttt tttcatttca ccaacatgta |
| 781 | ttattatctc acatttgaat ctaaatagta acacaacaat atcagcacaa accaattaca |
| 841 | tatttttcgt attataatat atttttttca tatcgattac aatcttaacg tcgttttata |
| 901 | aaataaattt ggggtttttt tttgttaaag ggttttaaaa caaaatttgt tccaagttaa |
| 961 | atgtcgttca aaaatttaat ggaatatata tatatatata tatatatttt tagaaaacac |
| 1021 | tagttataga attaaaatgg ataaaaatat gttattttaa ttgaacatat atacatcgaa |
| 1081 | actttttgtt ggttttgtta gcgtttagcg atgttgagct acgagttcta ttgatggttg |
| 1141 | tttacaacaa taattggatt ggagaacaag aagttataca tgattcgtga agttaattag |
| 1201 | ataagttttt aatacgaaga aatgagtccc gagacaaaaa tgaagcttat ggaattaatt |
| 1261 | ggtaaattag catggcgaca tacatttgtg ttatgaaatc atctagttgt aggcacggtg |
| 1321 | atggatccct cagatggtca tgctatcatt ttcgctttca aatagcgcga cctaattttt |
| 1381 | tatataataa aattactaac gtggatcgca tgggatattt taatataata aaaatgtttt |
| 1441 | aagaaaataa ggaaatggaa gagcccaccg tccaccaata aattaccgag taaacgattt |
| 1501 | atacgaccgt cgaaatgaac tgagaagata acgagaaaaa aagaatcgga attatatatt |
| 1561 | ttgactcaaa aacgagaaaa taattcgtag cgattctaac tcctacttta taccttaagg |
| 1621 | aacacgaaac ttatgagatt ttatggaagt tacaacgtgg ttagtttttt tttctttcta |
| 1681 | ttggaccagt gttaaatttt caatttggca tggtgtaaaa ctacacaaaa cagcctttct |
| 1741 | ttctctgacc cgtaaaacta ctattttatc ttatttcaaa tctaacagat tttcattatg |
| 1801 | gcgatagata tagtccttaa aaattatatt ggattcatta gcaaaacata actatacatt |
| 1861 | gaaattgtat tgataaaatt tatattatta catgcaacca agcaagagcg gatgtacacg |
| 1921 | ttttggtgtg ggtgcgagtt ccacatcaga atttgtttgt ctatataagt aattgtgaga |
| 1981 | gacaatcgga ataattggct agaatcagtc tttttttcct agtggatctt taaaaaccat |
| 2041 | tcttttatac caagcatgta catgctgtgg tgtgggtgta agtaaatcct gccccaatga |
| 2101 | aaattgtttt tggactcgcc actgcaacga agtgtaccaa caacttgact aggattctaa |
| 2161 | gttcttttat gtataggatg tctatattaa actaccatga ctaacatata tatagtagtt |
| 2221 | ccatatgctc gataaactat gatagatcaa caattttaaa catatagttt aacactattt |
| 2281 | atttgttcaa cgtcaatagt ttatagttcg catgcgctcg gcttagattt ggtccccaac |
| 2341 | agtcgaaatt gtcaaataat ataaaataaa agtttcattg ttaggattca tttattcttc |
| 2401 | gggtggttat tgtaataaaa ggcaaaagaa aaagaagaac aaaattcaca agtaaaaaaa |
| 2461 | aagataacat cattctttta gtcgacaaaa aaaaaaaaaa aatcaaaaag atttattcag |
| 2521 | tactacagtt taatattgtt ttgacttttt tctttttctt tatattatct gaaaattcta |
| 2581 | gactgcagct gaaacatgtg atatggatta aaggcgtatc cagtatccac agaaagagga |
| 2641 | gtggtgtcgc tcacccagtc acccttgtta cttgttagat agcattaata catttgtaag |
| 2701 | caacagctta tctaatagac atgtcttaat tgggaaatat gctctaagat gatacaacca |
| 2761 | tggttccaac tgttgaccac cataactgat aacatgttga ttacattttt tcttttcagt |
| 2821 | tacaacgatt acttttttgg ggaaattatt gatataatat gattcattgg atgatccgat |
| 2881 | atcatgcata taaagttgta tctcgtgaaa cacgagatag tattatactc cattctttca |
| 2941 | ttatcggagt atgtttaaaa tttgaaaaca aatacagaca cggaccgtgg tctttacctt |
| 3001 | cagaaaaaaa aagagaaaaa aaaacaatcc actgtttatt ataggagttg tagaaaatcg |
| 3061 | ggcaacgata ttcgatatga gttattatta gggccttatt attatatggt attactggat |
| 3121 | attactaaat aatcatataa atatcacatt ttaatataca ctcgttggac acgcggaata |
| 3181 | ttatatgttc taaatgttaa aaaatcaaca gaatacaacg atcgacggat ctagagtcta |
| 3241 | gaccatgcaa atacctcatc ctatttacat ataataactg tgcatatagt ttagtcaaat |
| 3301 | aaaaaggtaa agaaacaata tacaacctat aacgtcaata tccatgtacg tagtaataat |
| 3361 | taggatatga cacgaacaca cgatatcttg atatatacaa aatgaaaact taaaaattga |
| 3421 | ttaatatggc ctggctgggt atattattaa aaaaacataa agagagatca ataattgatt |
| 3481 | cgaagatcac tatataaaga acgtcttcga tatgtaaaag aaccatccta aacatttttt |
| 3541 | cttgaataaa atcagaatta caaacaaaa |

SEQ ID NO: 4 (promoter sequence of malate synthase gene from Brassica napus)

| | |
|---|---|
| GAGAGAGGATCCAGAGATTATCAACACGTGGGAGCTTATGGAAGATCTCGAAGATTCAAC | 60 |
| GAAGATTAGTCCCAAATCTCGTGGGATCTTCGGGAAATCATGGAAGACTCCGGTGAAATC | 120 |
| GATTGTTGAATCTCCTAAGAGGAATGGTAGTAGTAAGAGATTCAGGGGAAAAGAAAACAG | 180 |
| AGGAGAGAAACAGAGTCCGAACCAGATTCTGAAGACTCCAAAGAGAGGCGTGATGCGTTT | 240 |
| GAGTTTCCTCTACAAATCAGAAGAGATTACGCAGAGGAGGAGGAAGAGTTTCAGTCCAAT | 300 |
| GTTCGATCCAGACCTCGTGGCTTCTTACGAGAGGGAGTTGTCTCAGGAGAAAGAACAGAT | 360 |
| CAAGATGGTGATCTCTCCTCCAGACCCTCTCCCGGAGAATGTCCGCCGGGAGGAGAGAAC | 420 |
| TCGGTGGTCGTCTACATAACGACGCTGAGAGGGATCAGGAAGAGCGTTCGAGGACTGCAA | 480 |
| CGCGGTGAGATCGATACTGATTCGCACGAGGTTCGGTACTCGGAGAGGGATGTGTCGATG | 540 |
| CACTCTGTTTTCAAGGAGGAGATTAGAGGGATCATGGGGACGAAGCAGGTGAAGATACCG | 600 |
| GCGGTTTTCGTGAAGGGTAGGATGATAGGAAGCGTTGAGGAAGTTGTGAGGTTGGAGGAG | 660 |
| GAGGGTAAATTGGGTATTTTGCTTGAGTGTATGCCTAAGGCGAGGGTAAGCGGTTGCTGC | 720 |
| TGCTGCGGGTGCGGTGGGATGAGGTTTGTGATGTGTGGGGTTTGTAATGGAAGGCTGCAA | 780 |
| GGTTAGGGATGCGGAGAAGAAGGATACGGTTAAGTGTTTGGAGTGTAATGAGAATGGTTT | 840 |
| GGTTGTTTGTCCAATGTGTTCGTAAAAGAGGTTTCTTCTTTTTCAGTTTTGTCCTAATTT | 900 |
| TGTTGTGAAAATTGGGTGAGACTGTAAGAGGGTTGACTTAACTTTGGAGGCTAACTTTTT | 960 |
| GCATTTGAATCTTGATGGGTAGAATCTAATGATTTGTGAGAGAGTTTCTAAAGTTGGGTT | 1020 |
| TAATGTTTCTTGGTGTGTACTAGTAACTGAATCTGTGGTTTAATGTTTGTAAACGTTTTT | 1080 |
| ATAATAAAGATTCAATTTATTTTGTATAACCATCGTAAATATCGTTTTGTTTGATTCTTT | 1140 |
| CTTCAATGTCTACTATTTTATTTTTTGATAAAATGTTAATTATCATGACAAGTTTTTGAT | 1200 |
| TTGTGACAGAAATTACAAAAATAACTAGTAGCAAAATGATGTAAATAAACTAAACACCAA | 1260 |
| TACTAACGGTTATGAACTCAGCCTGGTATAATAATCTTTCTTCAATGGTCTAATATGATT | 1320 |
| AACCACATATTCTTTCTTCATTGGTTCTAAAGATGCTAAAGTTTGGTTTGACTACTAACT | 1380 |
| AGTAATTGCAACTGCTTTTAATGTGTGTAAGCGTTTTTATACATGATTCAATTTATTCTT | 1440 |
| CAGTGGTCTTATATGACAAACCACCTATTCTTTCTTCATTGAGTTTATTTGCAATATGAA | 1500 |
| GCTAATAAACTTTTGTTTATAGTGATGATCATCAGATTAAAAAATATAACGAATAAAGAA | 1560 |
| AAAATAGATAAAAAAATTTGAAAAAAAAACAAATAGATAAATTTTGAGATAATCATAACC | 1620 |
| GATATGATGACGAAAAGCATACATAACTTGGCACACATCCCCAAATGATCCCTGAATCTC | 1680 |
| AGGCACACATGTCAATGCATATCCCCTATCCATTTCCACCTTTATAATTCATAACATCCG | 1740 |
| ACGATGATTTTTATTCACATATAACAAAAATAACAAAAGCCAAAAAATG | 1789 |

SEQ ID NO: 5 (promoter sequence of isocitrate lyase gene from Brassica napus)

| | |
|---|---|
| TGTCCTTTATTGCTTTTCAGTAGCATTCTAATTCAAAAATCATCTCACCCATTGATAGCA | 60 |
| TTTAGATTAAGCATGGTCTTACATTCCCTTTGCTTTAGAATCACTTAGAACTTATTTGAC | 120 |
| ATCTTTTATTCTACAACATTTGATTAAGAGCCTTGAAACTCCTATCATCATTCCTCAGAT | 180 |
| CGTTCATGATAATGCTTTGAGTCCAGCCGTTTCAGGATCACGTAAGAAGCAAAAGACATC | 240 |
| ACAATCAATGGCCTCATTAGCGATGGGCCCTCCATCTCCTGCTATGCAACCATCTTCTTC | 300 |
| TGCGCTAAGAAGGGGAGGTCTTTCACCAGGTATCTTATCTTTCATTCCTACACAGTCAAG | 360 |
| TGACAATGTGCTTTAGTGTCTAGAGTTAATGGAGGTGTTGTTGATGCAATTTGCTTGGAT | 420 |
| CATAAGGTTTTAATGCATAGAATGAAAGAAATTAAGAACCCCTAAATATGAGACTCCCAT | 480 |
| TAGAGCACAAAATCAAAGTGTTTCTTAACTAAAGTTCTTAATTACATTTAAATACTAAAA | 540 |
| TAATCACTAAGAGATCCTAAGTGGGGTTGTGGGTTAATCATGCTCTTATTATTCCAATTT | 600 |
| AAGAGGCTTTTTTTTGTTTTAATTGCTTTTTTTCTTTTTTTAATCATAATTTCATCTAAG | 660 |
| AACCCCTTAAGATACATGGATAATGATGCTTTGAGAACATGATTATTGGAAGGTTCTTAA | 720 |
| GGTGGGATTCTTAGCGGAATATAAGAATCTGACTCTTAATTTTTAATTAAAAAGACTAAG | 780 |
| AACCGGCTCTTAAATAATAGTTTTAAGAGACGGTTCTTACCTAGGAATCTTTGCGATCAG | 840 |
| TCAATAGGCCCATTGGGTCCCAATCGTTGTCTAAGTTGCCTTTAAGTTTGAAACCCATTG | 900 |
| ACCGATTTTTATTCTAGTTATTTTTTTGTCCACCATTAACTCGTTCCTTCATGGCCCTAA | 960 |
| CTTTTGGTCGCTGGATGACTATCTTTTTTCTCTATGTTTCATATTGCTAGTTGCATTGAT | 1020 |
| AAGATATTATCTTGAGCAGGCAAGTAGACAACTGTTTTGATCCTCCAGAAAAAAGTAAAG | 1080 |
| AAAAGGCAGCAACACGAAAAGATTATATAACAATTCCAAAAGATAGATGCCTAAAATAAA | 1140 |
| AGTCTATAAGCACATCTATTAATAAAATTTCAAAGTAACTGAAATGTATTCGTAATCTCG | 1200 |
| TGAAGTATTTGAACATGTATGTTATATTGATACATGTGTTGGCTTGAGGTTCATCGAATG | 1260 |
| GCTAAATCCGAATGTGTTATTGAGCTAATATCATTGACAGAGTTCTAATCGTAAAAGCCC | 1320 |
| ATCCTGTGGCACACCTTCCAATGTGCAAGTTGCAATATAAAATCTGTTGTCATTATATAA | 1380 |
| TTCGATTTTCCCTTGAGAAAATAATTTATCTGTTAATAGTGACGTAGTCCCCGCCGCGAC | 1440 |
| CAAGTAGCGTCCTGATTTCATCAACGACCGGTGATAAGTACTTAAATCGCTCTAACATAT | 1500 |
| TACTTCCTCCCATATAAGATATATCCGATCTGATGGTTAAAATAATATTTATAAATTATA | 1560 |
| AGCAAAACATCATATTTATAAATTATAAGCAAAACATCCCAGTTTTGTTACAAATACTCG | 1620 |
| AGTCTGGTTTAAGTTAAGATCAGAATTCCCGGTTTCTATTTGTTTTTTTTTTAATAAATT | 1680 |
| TTTTCAGTAACCCGTTTCCTTTAAGTCAAGTCAAACTGTATAATTAGTCCTATTATTTTT | 1740 |
| ATAAGCAAATACCAGAAAATGCCAACATCCAAATTGGAAAGATAGGATTGCCAAGTCGCA | 1800 |
| TGCAATGTGCAAATCCATTCAAAGCAAGATAGGGTTTATCTTTTTTCCTCGGGAAACATA | 1860 |
| ACTATTTGTTTTGAAACTTTTTTCCCACGTTAAAGGTACGATTTTAAAAAGTTTACCCAT | 1920 |
| TTATACAATAAGTACCAGTATTTTTTTTTGGTAAAAGAAAAAGAACCCAAATAGCACTTA | 1980 |
| GAAAAATTTAATCGAATGAGAATGTTTAGTTCATATCTTCAAACATCAATATCGAATTTA | 2040 |
| TTGAGACCTAGCAGCATGAATTAGCAATGGCTAGTCCAGATAGTGCATGGATCTAAGAGA | 2100 |
| TTGAACTTGGATTTCACAAAGATGTAATATAATGTGTTTTATACGAAAAAGGAAGACTAA | 2160 |
| TATAACGTAAGAAATTTTTGTAAAATGTAGATCAGTTTTACCATAATGAGAATGGTAAAA | 2220 |
| CTGATCTACATTTTGCAAGTTTATTGATCAAAAACGTGTGAGGTTGGTAAGACGACACGT | 2280 |
| TTGAAGCAATTGTGCATGTATAGAATTCTCTGGAAGACTAATATAACGTAAGAAATTTTT | 2340 |
| GTTCTTTGTTAGTTAATTAATTACACCCACTTTGCCGACAGATATACTCTTTCCGATGAA | 2400 |
| GTTTGTGATTATTGCTTAACTGATTATTAGATCATATGGCAACAAGATCTTAGGCAATGC | 2460 |
| ATACTTACATGGACCAGCACATATATCTGACCGTAACTGTATCCTAAATCCTTGTGCAAC | 2520 |
| TCTGGTTCGCACTAAACTATATACACTTCAGATTTGTTTTCTAGAATGAGATCAACAGAA | 2580 |
| AGAGACGATAAAGGATGTCTGATGATAATTCGGTTCTGCCATAATTCACTCGTACGTGTC | 2640 |
| ACAATTATTCAAGTTTTGAGAAAACAAAAATTTAAGCCAACTAGAGAAAGACATATACAC | 2700 |
| CAGCACAAGTAACTTTTTCAGTAAAATAGTTTTAAATACTTTATTTTAAAATATTTTTTT | 2760 |
| TAATTCAGAAACTAGTATATAAGGGGAAGAAAGGAAAGAAGAGCAACATGCTTGAAGTTT | 2820 |
| CTCTACTTTCATAAGTCCTTAAAAGAAAAATCATTTCCAATTCATAAAATTTGAAGCCAT | 2880 |
| GG | 2882 |

SEQ ID NO: 6 (promoter of a glycine-rich RNA binding protein gene from
Oryza sativa, minimal promoter sequence)
tgtggtgggccgcggcggcccataaaagaaatatctaggcggcccatgtagcgccagaaa
atatcttctcccccgcctcgggatccttatcctccgcctcgcgcggggtgccgtccgatc
agatcaggacggccgcgtggggctataaaaggagggggggtagggcaagcatgtcctcct
SEQ ID NO: 7 (promoter of a glycine-rich RNA binding protein gene from
Oryza sativa,, full promoter sequence.)

| | |
|---|---|
| −1730 | tagcttctaataattgttagtaggtatcaatagattgtttaatttaactg |
| −1680 | gccatggaaagaatggtattggcatcaatggcatgaccgtttctataaaacccttcttat |
| −1620 | tgatcaatgcatgatatctttaattaaatcccctttccctttttctcttctaaggtgatg |
| −1560 | tttggaaccagatacttaactttagtctatatatttagacactaatttagagtattaaat |
| −1500 | atagactacttacaaaactaattacataaatgaaagctaatttgcgagataaatttttta |
| −1440 | agcctaattaatctataattagagaatttttactgtagcatcatataggcatatcatgga |
| −1380 | ttaattaggctcaatagatttgtctcgcgaattagtccgagattatggatgagttttatt |
| −1320 | gatagtctacgtttaatatttataattagtgtccaaacatcccatgtaatagggacttaa |
| −1260 | aagttttagtcccatctaaacagggtctaagtccttctaaatctgttactcatataactg |
| −1200 | tctaactgagataaagtttaaggttgtcatatcatatcatcgtcacgttatatatatgat |
| −1140 | ccctgcacttctctttttatagaatggacgagactcttttttctgtatatgtagcggtct |
| −1080 | tgtactcttgttagtaccattttgcgtcccattttgacgagacgactggcgtgccatttt |
| −1020 | gcgtcctggttcattacagtctaatttggtgacaaacaaacaaggaacaaataggtccca |
| −960 | tggtctagcggttaggacattggactctgaatccagtaacccgagttcaaatctcggtgg |
| −900 | gaccttaattttctcggttttattttctgcctgagcttattgtcctcctcctgatttttt |
| −840 | gttgttgtctattttctctgccggaaaaatgtatcaaactcgtcgattctactcgtttga |
| −780 | gagcttactgtgatattgtccttctcctgaagtttctattttttactctctctgttatga |
| −720 | aaattttcatgctagaatgatttacattgtgaaatggagagagaactcgtttgtgcttat |
| −660 | ttatccttcccctgatttttttccacaccaaaacatatattgtgatggttgagtatgcta |
| −600 | cgcgtctgacgtactacgagtttactccctccgtcccaaaaaaagacaaaccctgagttt |
| −540 | tcatgtccaatgtttgatcatattatttgaaaaaattatgaaaaaattaaaaagccagtt |
| −480 | acgtataaagtattaatcatattttatcatataacaacaatgaaaatactaattataaaa |
| −420 | atttttcatataagacggacagttaaacgttggacacgaaaatctaggatttattttttt |
| −360 | ttatagagggagtacgaggtaaaaatcgtcctcagcgccttcagaaaaaaaaaggacaaa |
| −300 | aatcctcagcgccaaccgactccgctccacagaccacagccgcccaagtgtgcgaggaca |
| −240 | acggcggcggcggcggcggctaggtttttgctgcacccgacgccaccgcccaccagcgag |
| −180 | tgtggtgggccgcggcggcccataaaagaaatatctaggcggcccatgtagcgccagaaa |
| −120 | atatcttctcccccgcctcgggatccttatcctccgcctcgcgcggggtgccgtccgatc |
| −60 | agatcaggacggccgcgtggggctataaaaggagggggggtagggcaagcatgtcctcct. |

1. A transgenic plant comprising a gene that confers resistance to oxidative stress in said plant, wherein said gene encodes an enzyme that has anti-oxidant activity or an enzyme that produces an antioxidant compound, and wherein said gene is under the control of a regulated promoter functional during the early ontogeny of said plant.

2. Transgenic plant according to claim 1, wherein said oxidative stress is caused by drought, temperature, radiation, salt and/or exposure to reactive oxygen species.

3. Transgenic plant according to claim 1, wherein said regulated promoter is an inducible promoter, a developmentally regulated promoter, and/or a tissue-specific promoter.

4. Transgenic plant according to claim 3, wherein said regulated promoter does not express said gene after the early ontogeny of said plant.

5. Transgenic plant according to claim 3, wherein said inducible promoter is a member selected from the group consisting of the alcA/alcR gene switch promoter, the GST promoter, an ozone inducible promoter and the ecdysone switch system.

6. Transgenic plant according to claim 3, wherein said developmentally regulated promoter is an ontogenesis-specific promoter selected from the group consisting of the Pyk10 promoter from Arabidopsis thaliana, the malate synthase promoter from Brassica napus, the isocitrate lyase promoter from Brassica napus, the promoter of the GSBF1 gene from Brassica napus, the glycine-rich RNA binding protein gene of Oryza sativa, the cysteine protease gene promoter of Brassica napus, the promoters of lipid transfer protein genes from Hordeum vulgare, and homologues thereof in other plant species.

7. Transgenic plant according to claim 3, wherein said tissue-specific promoter is a member selected from the group consisting of the promoter of the isoprene synthase gene from Populus alba, the rbcS (Rubisco) promoter from Coffea, Brassica, Chrysanthemum, Phaseolus; and Glycine max, the cy-FBPase promoter, the promoter sequence of the light-harvesting chlorophyll a/b binding protein from Elaeis, the STP3 promoter from Arabidopsis thaliana, the promoter of the PAL2 gene from Phaseolus, the enhancer sequences of the ST-LS1 promoter from Solanum tuberosum, the CAB1 promoter from Triticum, the stomata-specific promoter from the ADP-glucose-phosphorylase gene from Solanum tuberosum, the LPSE1 element from the P(D540) gene of Oryza sativa, and the stomata specific promoter pGC1(At1g22690) from Arabidopsis thaliana, and homologues in other plant species.

8. Transgenic plant according to claim 1, wherein said enzyme that produces an antioxidant compound is a member selected from the group consisting of isoprene synthase, glutathione reductase, dehydroascorbate reductase, L-galactono-γ-lactone dehydrogenase, phosphomannomutase, GDP-D-mannose pyrophosphorylase (GMP), GDP-mannose-3′,5′-epimerase, L-galactono-1,4-lactone dehydrogenase, Gal-UR, the gene encoding miox4, and L-idonate dehydrogenase.

9. Transgenic plant according to claim 1, wherein said enzyme that has anti-oxidant activity is a member selected from the group consisting of glutathione peroxidase, glutathione reductase, catalase, thioredoxin reductase, superoxide dismutase, heme oxygenase and biliverdin reductase.

10. Transgenic plant according to claim 1, wherein said early ontogeny is the prefloral stage or the vegetative stage.

11. Transgenic plant according to claim 1, wherein said gene is expressed in the plastids of said plant.

12. Transgenic plant according to claim 1, wherein the gene is the isoprene synthase gene.

13. Transgenic plant according to claim 12, wherein the expression of said vector in said plant results in an terpene emission rate of at least 0.1-200 nmol·m⁻²·s⁻¹.

14. Transgenic plant according to claim 12, wherein said plant is from a species that does not naturally emit isoprene.

15. Transgenic plant according to claim 1, wherein said plant as an adult plant exhibits a first rate of respiration under ambient ozone that is essentially equal to the rate of maintenance respiration exhibited by said plant during early ontogeny, and wherein said plant upon temporary exposure to ambient plus 100 ppb of ozone exhibits a second rate of respiration, which second rate is a significant increase relative to said first rate, and wherein following said temporary exposure said second rate of respiration returns to pre-exposure levels.

16. A progeny plant or seed from the transgenic plant of claim 1, wherein said progeny plant or seed comprises said gene under the control of said regulated promoter.

17. A seed from the progeny plant of claim 16, wherein said seed comprises said gene under the control of said regulated promoter.

18. A plant from the seed of claim 17, wherein said plant comprises said gene under the control of said regulated promoter.

19. A method of preparing a transgenic plant having improved yield under conditions of periodic oxidative stress, said method comprising the steps of:
(a) obtaining a nucleic acid segment comprising a gene that encodes an enzyme that has anti-oxidant activity or an enzyme that produces an antioxidant compound, and wherein said gene is operably linked to a regulated promoter functional during the early ontogeny of said plant;
(b) transforming a plant cell with said nucleic acid segment; and
(c) regenerating from said plant cell a transgenic plant which expresses said gene and wherein said transgenic plant exhibits improved yield under conditions of periodic oxidative stress as compared to a non-transformed plant.

20. The method of claim 19, wherein step a) further comprises introducing said nucleic acid segment into a vector, and wherein step b) comprises transforming said plant cell with said vector.

21. The method of claim 20, wherein said vector is a phage vector, bacterial vector, a plasmid vector or viral vector.

22. The method of claim 19, wherein said oxidative stress is caused by drought, temperature, radiation, salt and/or exposure to reactive oxygen species.

23. The method of claim 19, wherein said regulated promoter is an inducible promoter, a developmentally regulated promoter, or a tissue-specific promoter

24. The method of any claim 23, wherein said regulated promoter does not express said gene after the early ontogeny of said plant.

25. The method of claim 23, wherein said inducible promoter is a member selected from the group consisting of the alcA/alcR gene switch promoter, the GST promoter, an ozone inducible promoter and the ecdysone switch system.

26. The method of claim 23, wherein said developmentally regulated promoter is an ontogenesis-specific promoter selected from the group consisting of the Pyk10 promoter from Arabidopsis thaliana, the malate synthase promoter from Brassica napus, the isocitrate lyase promoter from Brassica napus, the promoter of the GSBF1 gene from Brassica napus, the glycine-rich RNA binding protein gene of Oryza sativa, the cysteine protease gene promoter of Brassica napus, the promoters of lipid transfer protein genes from Hordeum vulgare, and homologues thereof in other plant species.

27. The method of claim 23, wherein said tissue-specific promoter is a member selected from the group consisting of the promoter of the isoprene synthase gene from Populus alba, the rbcS (Rubisco) promoter from Coffea, Brassica, Chrysanthemum, Phaseolus; and Glycine max, the cy-FBPase promoter, the promoter sequence of the light-harvesting chlorophyll a/b binding protein from Elaeis, the STP3 promoter from Arabidopsis thaliana, the promoter of the PAL2 gene from Phaseolus, the enhancer sequences of the ST-LS1 promoter from Solanum tuberosum, the CAB1 promoter from Triticum, the stomata-specific promoter from the ADP-glucose-phosphorylase gene from Solanum tuberosum, the LPSE1 element from the P(D540) gene of Oryza sativa, and the stomata specific promoter pGC1(At1g22690) from Arabidopsis thaliana, and homologues in other plant species.

28. The method of claim 19, wherein said enzyme that produces an antioxidant compound is a member selected from the group consisting of isoprene synthase, glutathione reductase, dehydroascorbate reductase, L-galactono-γ-lactone dehydrogenase, phosphomannomutase, GDP-D-mannose pyrophosphorylase (GMP), GDP-mannose-3′,5′-epimerase, L-galactono-1,4-lactone dehydrogenase, Gal-UR, the gene encoding miox4, and L-idonate dehydrogenase.

29. The method of claim 19, wherein said enzyme that has anti-oxidant activity is a member selected from the group consisting of glutathione peroxidase, glutathione reductase, catalase, thioredoxin reductase, superoxide dismutase, heme oxygenase and biliverdin reductase.

30. The method of claim 19, wherein said early ontogeny is the prefloral stage or the vegetative stage.

31. The method of claim 19, wherein said gene is expressed in the plastids of said plant.

32. The method of claim 19, wherein the gene is the isoprene synthase gene.

33. The method of claim 32, wherein the expression of said vector in said plant results in a terpene emission rate of at least 20 nmol·m⁻²·s⁻¹.

34. The method of claim 32, wherein said plant is from a species that does not naturally emit isoprene.

35. The method according to claim 33, wherein the terpene emission rate is an isoprene emission rate.

36. A transgenic plant obtained by the method according to claim 19, wherein said plant comprises said gene under the control of said regulated promoter.

37. A transgenic seed from the plant of claim 36, wherein said seed comprises said gene under the control of said regulated promoter.

38. A transgenic plant from the seed of claim 37, wherein said plant comprises said gene under the control of said regulated promoter and wherein said plant, when grown from said seed to maturity under periodic conditions that cause oxidative stress, exhibits a total dark respiration and/or respiration via the alternative oxidase (AOX) pathway that is significantly less as compared to a non-transformed plant.

39. The plant of claim 38, wherein said plant exhibits a rate of respiration via the alternative oxidase (AOX) pathway that is below 40% of the total dark respiration of said plant.

40. A method of growing plants, comprising the step of allowing a seed, a seedling, tissue culture or plantlet of the plant of claim 36 to develop into a plant, and inducing expression of said gene during early ontogeny of said plant or during periodic conditions that cause oxidative stress, to thereby prevent an increase in the rate of respiration via the alternative oxidase (AOX) pathway during said early ontogeny and/or due to said oxidative stress.

41. The method of claim 40, wherein said induction is brought about by contacting said seed, seedling, tissue culture, plantlet or plant with an effective concentration of an promoter-inducing agent.

42. The method of claim 40, wherein the early ontogeny of said plant is the prefloral stage or the vegetative stage.

43. The method of claim 19, wherein said plant is a plant selected from the group consisting of wheat, corn, melon, soy, potato, rice, sugarcane, sugarbeet, evening primrose, meadow foam, hops, jojoba, peanuts, safflower, barley, oats, rye, wheat, sorghum, tobacco, kapok, beans, lentils, peas, soybeans, rape, mustard, poppy, olives, sunflowers, coconut, castor oil plants, cocoa beans, groundnuts, cotton, flax, hemp, jute, cinnamon, tomato, cucumber, pepper, camphor, coffee, sugarcane, tea, and a natural rubber plant.

44. The method of claim 40, wherein said method further comprises the step of discontinuing said induction when said plant reaches the floral stage or the generative stage or when said periodic conditions that cause oxidative stress are absent.

45. The method of claim 40, wherein said method further comprises determining prior to or simultaneously to growing said seed, seedling, tissue culture, plantlet or plant:
(a) the total dark respiration and/or respiration via the alternative oxidase (AOX) pathway in said plant or a plant of the same variety;
(b) the length of the early ontogenic phase in said plant or a plant of the same variety; and/or
(c) the length and interval of the periodic conditions that cause oxidative stress;
and using said information in order to induce expression of said gene during early ontogeny of said plant and/or during said periodic oxidative stress during the early ontogeny of said plant, but not during maturity of said plant or during the generative phase.

46. Method according to claim 40, wherein said early ontogeny of said plant is the vegetative or prefloral stage.

47. The transgenic plant of claim 1 wherein the gene is a heterologous gene.

48. The transgenic plant of claim 1 wherein the reactive oxygen species is ozone.

49. The transgenic plant according to claim 10, wherein said early ontogeny is the period between germination and 0.5-6 months post germination.

50. The transgenic plant according to claim 12, wherein the expression of said vector in said plant results in a terpene emission rate of between 10-200 nmol·m⁻²·s⁻¹.

51. The transgenic plant according to claim 12, wherein said plant is a row crop plant that does not naturally emit isoprene.

52. The method of claim 19, wherein said early ontogeny is the period between germination and 0.5-6 months post germination.

53. The method of claim 32, wherein the expression of said vector in said plant results in a terpene emission rate of between 50-1000 nmol·m⁻²·s⁻¹.

54. The method of claim 32, wherein said plant is a row crop that does not naturally emit isoprene.

55. The plant of claim 38, wherein said plant exhibits a rate of respiration via the alternative oxidase (AOX) pathway that is below 30% of the total dark respiration of said plant.

56. The method of claim 40, wherein the early ontogeny of said plant is a period from 1-6 months post-germination.
